(19) United States
(12) Patent Application Publication
(10) Pub. No.: US 2007/0185104 A1
(43) Pub. Date: Aug. 9, 2007

(54) Benzoxazole acetonitriles

(75) Inventors: Matthias Schwarz, Geneva (CH); Pascale Gaillard, Collonges sous Saleve (FR); Patrick Page, Saint-Julien-en-Genevois (FR); Jean-Pierre Gotteland, Beaumont (FR); Russell J. Thomas, Siena (IT)

Correspondence Address: OBLON, SPIVAK, MCCLELLAND, MAIER & NEUSTADT, P.C., 1940 DUKE STREET, ALEXANDRIA, VA, 22314, US

(73) Assignee: APPLIED RESEARCH SYSTEMS ARS HOLDING N.V., Curacao (AN)

(21) Appl. No.: 10/571,323

(22) PCT Filed: Sep. 10, 2004
(86) PCT No.: PCT/EP04/52141

§ 371 Date: Aug. 25, 2006

(30) Foreign Application Priority Data
Sep. 12, 2003 (EP) 032102739.4

(51) Int. Cl. A61K 31/5377; A61K 31/506; C07D 413/14

(52) U.S. Cl. 514/232.8; 544/331; 544/122; 514/275

(57) ABSTRACT

The present invention is related to benzoxazole acetonitriles as well as to pharmaceutical formulations containing such benzoxazole acetonitriles pof formula (I). Said benzoxazole acetonitriles are useful in the treatment of metabolic disorders mediated by insulin resistance or hyperglycemia, comprising diabetes type II, inadequate glucose tolerance, insulin resistance, obesity, polycystic ovary syndrome (PCOS). The present invention is furthermore related to methods of preparing benzoxazole acetonitriles (I). A is a pyrimidinyl.L is a secondary or tertiary amino group, or a 3-8 membered heterocycloalkyl, containing at least one heteroatom. selected from N, O, S or L is an acylamino moiety. R¹ is selected from the group comprising or consisting of hydrogen, sulfonyl, amino, C₁C⁶-alkYl, C₂-C₆-alkenYl, C₂-C₆-alkynyl or C₁-C₆-alkoxy, aryl, halogen, carboxy, aminocarbonyl, cyano or hydroxy.

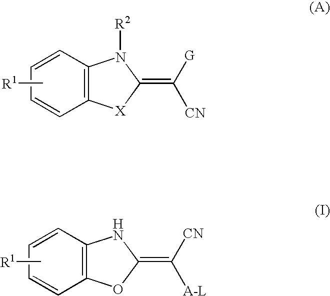

### FIELD OF THE INVENTION

The present invention is related to benzoxazole acetonitriles, as well as pharmaceutical compositions containing such benzoxazole acetonitriles. The compounds of the present invention are useful in the treatment of metabolic disorders mediated by insulin resistance or hyperglycemia, comprising diabetes type II, inadequate glucose tolerance, insulin resistance, obesity, polycystic ovary syndrome (PCOS). In one embodiment, the compounds of the present invention are inhibitors of Glycogen Synthase Kinase 3 (GSK3). The present invention furthermore relates to methods for the preparation of benzoxazole acetonitriles.

### BACKGROUND OF THE INVENTION

Diabetes mellitus is a serious metabolic disease that is defined by the presence of chemically elevated levels of blood glucose (hyperglycemia). The term diabetes mellitus encompasses several different hyperglycemic states. These states include Type 1 (insulin-dependent diabetes mellitus or IDDM) and Type 2 (non-insulin dependent diabetes mellitus or NIDDM) diabetes. The hyperglycemia present in individuals with Type 1 diabetes is associated with deficient, reduced, or nonexistent levels of insulin that are insufficient to maintain blood glucose levels within the physiological range. Conventionally, Type 1 diabetes is treated by administration of replacement doses of insulin, generally by a parenteral route.

Type 2 diabetes is an increasingly prevalent disease of aging. It is initially characterized by decreased sensitivity to insulin and a compensatory elevation in circulating insulin concentrations, the latter of which is required to maintain normal blood glucose levels. As described below, GSK3 inhibition stimulates insulin-dependent processes and is consequently viewed to be useful in the treatment of type 2 diabetes. Recent data obtained using lithium salts provides evidence for this notion.

The prevalence of insulin resistance in glucose intolerant subjects is well known. Reaven et al (American Journal of Medicine, 60, 80 (1976)) used a continuous infusion of glucose and insulin (insulin/glucose clamp technique) and oral glucose tolerance tests to demonstrate that insulin resistance exists in a diverse group of non-obese, non-ketotic subjects. These subjects ranged from borderline glucose tolerant to overt, fasting hyperglycemia. The diabetic groups in these studies included both insulin dependent (IDDM) and non-insulin dependent (NIDDM) subjects.

Coincident with sustained insulin resistance is the more easily determined hyper-insulinemia, which may be measured by accurate determination of circulating plasma insulin concentration in the plasma of subjects. Hyperinsulinemia may be present as a result of insulin resistance, such as is in obese and/or diabetic (NIDDM) subjects and/or glucose intolerant subjects, or in IDDM subjects, as a consequence of over injection of insulin compared with normal physiological release of the hormone by the endocrine pancreas.

The association of hyperinsulinemia and insulin resistance with obesity has been well established by numerous experimental, clinical and epidemiological studies (Stout, Metabolism, 34, 7 (1985)).

The association of hyperinsulinemia and insulin resistance with Polycystic Ovary Syndrome (PCOS) is also well acknowledged (Diamanti-Kandarakis et al.; Therapeutic effects of metformin on insulin resistance and hyperandrogenism in polycystic ovary syndrome; European Journal of Endocrinology 138, 269-274 (1998), Andrea Dunaif; Insulin Resistance and the Polycystic Ovary Syndrome: Mechanism and Implications for Pathogenesis; Endocrine Reviews 18(6), 774-800 (1997)).

Type II diabetes mellitus is currently treated with sulfonylureas, biguanides, such as Metformin and thiazolidenediones, such as Troglitazone, Rosiglitazone or Pioglitazone, as oral hypoglycemic agents.

Glycogen synthase kinase 3 (GSK3) is a serine/threonine kinase for which two isoforms, α and β, have been identified (Trends Biochem. Sci., 16 p. 177-81 (1991) by Woodgett et al.). Both GSK3 isoforms are constitutively active in resting cells. GSK3 was originally identified as a kinase that inhibits glycogen synthase by direct phosphorylation. Upon insulin activation, GSK3 is inactivated, thereby allowing the activation of glycogen synthase and possibly other insulin-dependent events, such glucose transport. Subsequently, it has been shown that GSK3 activity is also inactivated by other growth factors that, like insulin, signal through receptor tyrosine kinases (RTKs). Examples of such signaling molecules include IGF-1 and EGF. GSK3 beta activity is regulated by serine (inhibitory) and tyrosine (stimulatory) phosphorylation, by protein complex formation, and by its intracellular localization. GSK3 beta phosphorylates and thereby regulates the functions of many metabolic, signaling and structural proteins. Notable among the signaling proteins regulated by GSK3 beta are the many transcription factors, including activator protein-1 cells, Myc, beta-catenin, CCAAT/enhancer binding protein, and NFkappaB.

Agents that inhibit GSK3 activity are viewed to be useful in the treatment of type II diabetes.

In the patent literature, different classes of GSK3 inhibitors have been disclosed (e.g. WO 02/20495, Chiron Corporation; WO 02/10141, Pfizer Products Inc.; WO 02/22608, Vertex Pharmaceuticals Inc.).

WO 01/47920 discloses benzazoles of formula (A), in particular for the treatment of neuronal disorders, autoimmune diseases, cancer and cardiovascular diseases.

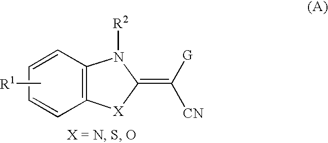

It was now found that certain compounds of formula (A); surprisingly, are in addition useful in the treatment of metabolic disorders mediated by insulin resistance or hyperglycemia, comprising diabetes type II, inadequate glucose tolerance, insulin resistance, obesity, polycystic ovary syndrome (PCOS).

### SUMMARY OF THE INVENTION

The present invention relates to benzoxazole acetonitriles of formula (I)

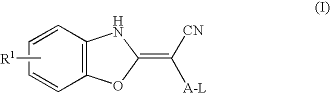

as well as their pharmaceutically acceptable salts.

Also, the present invention relates to the use of compounds of formula (I) as medicament, in particular for the treatment and/or prevention of metabolic disorders mediated by insulin resistance or hyperglycemia, such as diabetes type II, inadequate glucose tolerance, insulin resistance, obesity, polycystic ovary syndrome PCOS).

### DETAILED DESCRIPTION OF THE INVENTION

The following paragraphs provide definitions of the various chemical moieties that make up the compounds according to the invention and are intended to apply uniformly throughout the specification and claims unless an otherwise expressly set out definition provides a broader definition.

“C₁-C₆-alkyl” refers to alkyl groups having 1 to 6 carbon atoms. This term is exemplified by groups such as methyl, ethyl n-propyl, isopropyl, n-butyl, isobutyl, tert-butyl, n-butyl, n-pentyl, n-hexyl and the like.

“Aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenantrenyl and the like.

“C₁-C₆-alkyl aryl” refers to C₁-C₆-alkyl groups having an aryl substituent, including benzyl, phenethyl and the like.

“Heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

“C₁-C₆-alkyl heteroaryl” refers to C₁-C₆-alkyl groups having a heteroaryl substituent, including 2-furylmethyl, 2-thienylmethyl, 2-(1H-indol-3-yl)ethyl and the like.

“C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl (allyl, —CH₂CH═CH₂) and the like.

“C₂-C₆-alkenyl aryl” refers to C₂-C₆-alkenyl groups having an aryl substituent, including 2-phenylvinyl and the like.

“C₂-C₆-alkenyl heteroaryl” refers to C₂-C₆-alkenyl groups having a heteroaryl substituent, including 2-(3-pyridinyl)vinyl and the like.

“C₂-C₆-alkynyl” refers to alkynyl groups preferably having from 2 to 6 carbon atoms and having at least 1-2 sites of alkynyl unsaturation, preferred alkynyl groups include ethynyl (—C≡CH), propargyl (—CH₂C≡CH), and the like.

“C₂-C₆-alkynyl aryl” refers to C₂-C₆-alkynyl groups having an aryl substituent, including phenylethynyl and the like.

“C₂-C₆-alkynyl heteroaryl” refers to C₂-C₆-alkynyl groups having a heteroaryl substituent, including 2-thienylethynyl and the like.

“C₃-C₈-cycloalkyl” refers to a saturated carbocyclic group of from 3 to 8 carbon atoms having a single ring (e.g., cyclohexyl) or multiple condensed rings (e.g., norbornyl). Preferred cycloalkyl include cyclopentyl, cyclohexyl, norbornyl and the like.

“C₁-C₆-alkyl cycloalkyl” refers to C₁-C₆-alkyl groups having a cycloalkyl substituent, including cyclohexylmethyl, cyclopentylpropyl, and the like.

“heterocycloalkyl” refers to a C₃-C₈-cycloalkyl group according to the definition above, in which 1 to 3 carbon atoms are replaced by hetero atoms chosen from the group consisting of O, S, NR, R being defined as hydrogen or C₁-C₆ alkyl. Preferred heterocycloalkyl include pyrrolidine, piperidine, piperazine, 1-methylpiperazine, morpholine, and the like.

“C₁-C₆-alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like. “Carboxy” refers to the group —C(O)OH.

“C₁-C₆-alkyl carboxy” refers to C₁-C₆-alkyl groups having a carboxy substituent, including 2-carboxyethyl and the like.

“Acyl” refers to the group —C(O)R where R includes H, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acyl” refers to C₁-C₆-alkyl groups having an acyl substituent, including 2-acetylethyl and the like.

“Aryl acyl” refers to aryl groups having an acyl substituent, including 2-acetylphenyl and the like.

“Heteroaryl acyl” refers to heteroaryl groups having an acyl substituent, including 2-acetylpyridyl and the like.

“C₃-C₈-(hetero)cycloalkyl acyl” refers to 3 to 8 membered cycloalkyl or heterocycloalkyl groups having an acyl substituent.

“Acyloxy” refers to the group —OC(O)R where R includes H, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acyloxy” refers to C₁-C₆-alkyl groups having an acyloxy substituent, including 2-(acetyloxy)ethyl and the like.

“Alkoxy” refers to the group —O—R where R includes “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl alkoxy” refers to C₁-C₆-alkyl groups having an alkoxy substituent, including 2-ethoxyethyl and the like.

“Alkoxycarbonyl” refers to the group —C(O)OR where R includes “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl alkoxycarbonyl” refers to C₁-C₆-alkyl groups having an alkoxycarbonyl substituent, including 2-(benzyloxycarbonyl)ethyl and the like.

“Aminocarbonyl” refers to the group —C(O)NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl aminocarbonyl” refers to C₁-C₆-alkyl groups having an aminocarbonyl substituent, including 2-(dimethylaminocarbonyl)ethyl and the like.

“Acylamino” refers to the group —NRC(O)R′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. “C₁-C₆-alkyl acylamino” refers to C₁-C₆-alkyl groups having an acylamino substituent, including 2-(propionylamino)ethyl and the like.

“Ureido” refers to the group —NRC(O)NR′R″ where each R, R′, R″ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R′ and R″, together with the nitrogen atom to which they are attached, can optionally form a 3-8-membered heterocycloalkyl ring.

“C₁-C₆-alkyl ureido” refers to C₁-C₆-alkyl groups having an ureido substituent, including 2-(N′-methylureido)ethyl and the like.

“Carbamate” refers to the group —NRC(O)OR′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Amino” refers to the group —NRR′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R and R′, together with the nitrogen atom to which they are attached, can optionally form a 3-8-membered heterocycloalkyl ring.

“C₁-C₆-alkyl amino” refers to C₁-C₆-alkyl groups having an amino substituent, including 2-(1-pyrrolidinyl)ethyl and the like.

“Ammonium” refers to a positively charged group —N⁺RR′R″, where each R, R′, R″ is independently, “C₁-C₆-alkyl”, “C₂-C₆-alkenyl”, “C₂-C₆-alkynyl”, “C₃-C₈-cycloalkyl”, “heterocycloalkyl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R and R′, together with the nitrogen atom to which they are attached, can optionally form a 3-8-membered heterocycloalkyl ring.

“C₁-C₆-alkyl ammonium” refers to C₁-C₆-alkyl groups having an ammonium substituent, including 2-(1-pyrrolidinyl)ethyl and the like.

“Halogen” refers to fluoro, chloro, bromo and iodo atoms.

“Sulfonyloxy” refers to a group —OSO₂—R wherein R is selected from H, “C₁-C₆-alkyl”, “C₁-C₆-alkyl” substituted with halogens, e.g., an —OSO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyloxy” refers to C₁-C₆-alkyl groups having a sulfonyloxy substituent, including 2-(methylsulfonyloxy)ethyl and the like.

“Sulfonyl” refers to group “—SO₂—R” wherein R is selected from H, “aryl”, “heteroaryl”, “C₁-C₆-alkyl”, “C₁-C₆-alkyl” substituted with halogens, e.g., an —SO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyl” refers to C₁-C₆-alkyl groups having a sulfonyl substituent, including 2-(methylsulfonyl)ethyl and the like.

“Sulfinyl” refers to a group “—S(O)—R” wherein R is selected from H, “C₁-C₆-alkyl”, “C₁-C₆-alkyl” substituted with halogens, e.g., an —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C²-C₆-alkynylheteroaryl”, “C₁-C₆-alkyl cycloalkyl”, “C₁-C₆-alkyl heterocycloalkyl”.

“C₁-C₆-alkyl sulfinyl” refers to C₁-C₆-alkyl groups having a sulfinyl substituent, including 2-(methylsulfinyl)ethyl and the like.

“Sulfanyl” refers to groups —S—R where R includes H, “C₁-C₆-alkyl”, “C₁-C₆-alkyl” substituted with halogens, e.g., an —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Preferred sulfanyl groups include methylsulfanyl, ethylsulfanyl, and the like.

“C₁-C₆-alkyl sulfanyl” refers to C₁-C₆-alkyl groups having a sulfanyl substituent, including 2-(ethylsulfanyl)ethyl and the like.

“Sulfonylamino” refers to a group —NRSO₂—R′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonylamino” refers to C₁-C₆-alkyl groups having a sulfonylamino substituent, including 2-(ethylsulfonylamino)ethyl and the like.

“Aminosulfonyl” refers to a group —SO₂—NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl aminosulfonyl” refers to C₁-C₆-alkyl groups having an aminosulfonyl substituent, including 2-(cyclohexylaminosulfonyl)ethyl and the like.

“Substituted or unsubstituted”: Unless otherwise constrained by the definition of the individual substituent, the above set out groups, like “alkyl”, “alkenyl”, “alkynyl”, “aryl” and “heteroaryl” etc. groups can optionally be substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “ammonium”, “acyl”, “acyloxy”, “acylamino”, “aminocarbonyl”, “alkoxycarbonyl”, “ureido”, “carbamate”, “aryl”, “heteroaryl“, “sulfinyl”, “sulfonyl”, “alkoxy”, “sulfanyl”, “halogen”, “carboxy”, trihalomethyl, cyano, hydroxy, mercapto, nitro, and the like. Alternatively, said substitution could also comprise situations where neighboring substituents have undergone ring closure, notably when vicinal functional substituents are involved, thus forming, e.g., lactams, lactons, cyclic anhydrides, but also acetals, thioacetals, aminals formed by ring closure for instance in an effort to obtain a protective group.

“Pharmaceutically acceptable salts or complexes” refers to salts or complexes of the below-identified compounds of formula (I) that retain the desired biological activity. Examples of such salts include, but are not restricted to acid addition salts formed with inorganic acids (e.g. hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, methanesulfonic acid and poly-galacturonic acid. Said compounds can also be administered as pharmaceutically acceptable quaternary salts known by a person skilled in the art, which specifically include the quaternary ammonium salt of the formula —NR,R′,R″⁺Z⁻, wherein R, R′, R″ is independently hydrogen, alkyl or benzyl, C₁-C₆-alkyl, C₂-C₆-alkenyl, C₂-C₆-alkynyl, C₁-C₆-alkyl aryl, C₁-C₆-alkyl heteroaryl, cycloalkyl, heterocycloalkyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

“Pharmaceutically active derivative” refers to any compound that upon administration to the recipient, is capable of providing directly or indirectly, the activity disclosed herein.

“Enantiomeric excess” (ee) refers to the products that are obtained by an asymmetric synthesis, i.e. a synthesis involving non-racemic starting materials and/or reagents or a synthesis comprising at least one enantioselective step, whereby a surplus of one enantiomer in the order of at least about 52% ee is yielded.

A first aspect of the invention consists in benzoxazole acetonitriles of formula I:

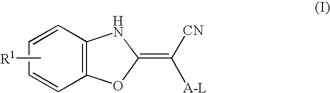

A is an unsubstituted or substituted pyrimidinyl.

In particular, A may be either of the substituted pyrimidinyl moieties

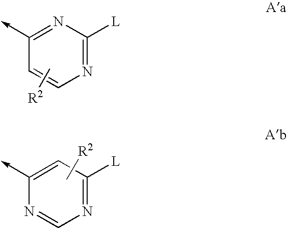

L is an amino group, or an unsubstituted or a substituted 3-8 membered heterocycloalkyl, containing at least one heteroatom selected from N, O, S or L is an acylamino moiety.

R¹ is selected from the group comprising or consisting of hydrogen, sulfonyl, amino, carboxy, aminocarbonyl, unsubstituted or substituted C₁-C₆-alkyl, unsubstituted or substituted C₂-C₆-alkenyl, unsubstituted or substituted C₂-C₆-alkyl or C₁-C₆-alkoxy, unsubstituted or substituted aryl (e.g. phenyl), halogen, cyano or hydroxy.

Preferably R¹ is H or C₁-C₃ alkyl (e.g. a methyl or ethyl group).

R² is selected from the group consisting of H unsubstituted or substituted C₁-C₆-alkyl, unsubstituted or substituted C₂-C₆-alkenyl, unsubstituted or substituted C₂-C₆-alkynyl. In particular R² may be a C₁-C₆-alkyl, e.g. a methyl or ethyl moiety.

Formula (I) also comprises its tautomers, its geometrical isomers, its optically active forms as enantiomers, diastereomers and its racemate forms, as well as pharmaceutically acceptable salts thereof. Preferred pharmaceutically acceptable salts of the formula (I) are acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

More specifically, the benzoxazole acetonitriles of the invention comprise the tautomeric forms, e.g. the below ones:

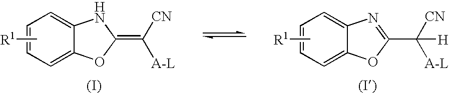

A specific embodiment of the present invention consists in benzoxazole acetonitriles of formula (Ia) in its tautomeric forms, e.g. the below ones:

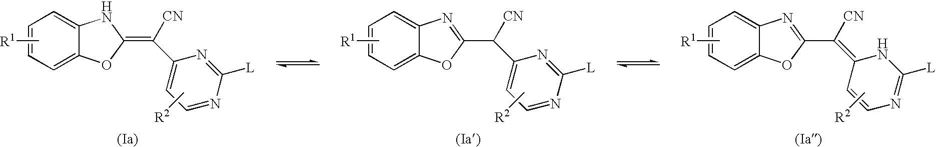

R¹, R² and L are as defined for formula (I).

According to a specific embodiment, the moiety L is an amino group of the formula —NR³R⁴ wherein R³ and R⁴ are each independently from each other H, unsubstituted or substituted C₁-C₆-alkyl, unsubstituted or substituted C₂-C₆-alkenyl, unsubstituted or substituted C₂-C₆-alkynyl, unsubstituted or substituted C₁-C₆-alkoxy, unsubstituted or substituted aryl, unsubstituted or substituted heteroaryl, unsubstituted or substituted saturated or unsaturated 3-8-membered cycloalkyl, unsubstituted or substituted 3-8-membered heterocycloalkyl, (wherein said cycloalkyl, heterocycloalkyl, aryl or heteroaryl groups may be fused with 1-2 further cycloalkyl, heterocycloalkyl, aryl or heteroaryl group), unsubstituted or substituted C₁-C₆-alkyl aryl, unsubstituted or substituted C₁-C₆-alkyl heteroaryl, unsubstituted or substituted C₁-C₆-alkenyl aryl, unsubstituted or substituted C₁-C₆-alkenyl heteroaryl, unsubstituted or substituted C₁-C₆-alkynyl aryl, unsubstituted or substituted C₁-C₆-alkynyl heteroaryl, unsubstituted or substituted C₁-C₆-alkyl cycloalkyl, unsubstituted or substituted C₁-C₆-alkyl heterocycloalkyl, unsubstituted or substituted C₁-C₆-alkenyl cycloalkyl, unsubstituted or substituted C₁-C₆-alkenyl heterocycloalkyl, unsubstituted or substituted C₁-C₆-alkynyl cycloalkyl, unsubstituted or substituted C₁-C₆-alkynyl heterocycloalkyl.

Alternatively, R³ and R⁴ may form a ring together with the nitrogen to which they are bound. This includes piperazines, piperidines, pyrrolidines or morpholines.

In a specific embodiment, R³ is hydrogen or a methyl or ethyl or propyl group and R⁴ is selected from the group consisting of H, unsubstituted or substituted (C₁-C₆)-alkyl, unsubstituted or substituted C₁-C₆ alkyl-aryl, unsubstituted or substituted C₁-C₆-alkyl-heteroaryl, unsubstituted or substituted cycloalkyl, unsubstituted or substituted heterocycloalkyl, unsubstituted or substituted aryl or heteroaryl and unsubstituted or substituted 4-8 membered saturated or unsaturated cycloalkyl.

In a even more specific embodiment R³ is H and R⁴ is selected from the group consisting of C₁-C₆ alkyl, 3-8 membered cycloalkyl, 3-8 membered heterocycloalkyl, aryl, heteroaryl, C₁-C₆-alkyl aryl, C₁-C₆-alkyl heteroaryl, C₁-C₆-alkyl cycloalkyl, C₁-C₆-alkyl heterocycloalkyl. Examples of cycloalkyl are cyclopropyl, cyclopentyl or cyclohexyl.

More specifically, R⁴ may be a C₂-C₄ alkyl, in particular an ethylene or propylene moiety, optionally substituted with an unsubstituted or substituted heteroaryl or heterocycloalkyl group, e.g., an unsubstituted or substituted pyridyl or a 2-pyrrolidinone (2-oxopyrrolidine) or a triazolyl moiety; or R⁴ is a C₂-C₄ alkyl, in particular an ethylene or propylene moiety, substituted by a unsubstituted or substituted heteroaryl or heterocycloalkyl-acyl group (—CO-heteroaryl (or heterocycloalkyl)). An example of this embodiment is where R⁴ is an unsubstituted or substituted propylene-CO-piperazino moiety.

According to a further specific embodiment, the moiety L is an acylamino moiety of the formula —NR³C(O)R⁴ wherein R³ and R⁴ are each independently from each other H, unsubstituted or substituted C₁-C₆-alkyl, unsubstituted or substituted C₂-C₆-alkenyl, unsubstituted or substituted C₂-C₆-alkynyl, unsubstituted or substituted C₁-C₆-alkoxy, unsubstituted or substituted aryl, unsubstituted or substituted heteroaryl, unsubstituted or substituted saturated or unsaturated 3-8-membered cycloalkyl, unsubstituted or substituted 3-8-membered heterocycloalkyl, unsubstituted or substituted C₁-C₆-alkyl aryl, unsubstituted or substituted C₁-C₆-alkyl heteroaryl, unsubstituted or substituted C₁-C₆-alkenyl aryl, unsubstituted or substituted C₁-C₆-alkenyl heteroaryl, unsubstituted or substituted C₁-C₆-alkynyl aryl, unsubstituted or substituted C₁-C₆-alkynyl heteroaryl, unsubstituted or substituted C₁-C₆-alkyl cycloalkyl, unsubstituted or substituted C₁-C₆-alkyl heterocycloalkyl, unsubstituted or substituted C₁-C₆-alkenyl cycloalkyl, unsubstituted or substituted C₁-C₆-alkenyl heterocycloalkyl, unsubstituted or substituted C₁-C₆-alkynyl cycloalkyl, unsubstituted or substituted C₁-C₆-alkynyl heterocycloalkyl.

Specific benzoxazole acetonitriles according to formula (I) include:

1,3-benzoxazol-2(3H)-ylidene(2-chloro-6-methylpyrimidin-4-yl)acetonitrile

1,3-benzoxazol-2(3H)-ylidene(2-chloro-6-methylpyrimidin-4-yl)acetonitrile

1,3-benzoxazol-2(3H)-ylidene(6-chloropyrimidine-4-yl)acetonitrile

1,3-benzoxazol-2(3H)-ylidene(2-chloro-5-metylpyrimidin-4-yl)acetonitrile

1,3-benzoxazol-2(3H)-ylidene(2-{[3-(2-oxopyrrolidin-1-yl)propyl]amino}pyrimidin-4-yl)acetonitrile

1,3-benzoxazol-2(3H)-ylidene(2-{[3-(1H-pyrazol-1-yl)propyl]amino}pyrimidin-4-yl)acetonitrile

1,3-benzoxazol-2(3H)-ylidene(2-{[2-(1H-1,2,4-triazol-1-yl)ethyl]amino}pyrimidin-4-yl)acetonitrile

1,3-benzoxazol-2(3H)-ylidene(2-{[2-(1H-pyrazol-1-yl)ethyl]amino}pyrimidin-4-yl)acetonitrile

1,3-benzoxazol-2(3H)-ylidene{2-[(2-pyridin-3-ylethyl)amino]pyrimidin-4-yl}acetonitrile

1,3-benzoxazol-2(3H)-ylidene[2-(cyclopropylamino)pyrimidin-4-yl]acetonitrile

1,3-benzoxazol-2(3H)-ylidene(2-{[3-(1H-1,2,4-triazol-1-yl)propyl]amino}pyrimidin-4-yl)acetonitrile

1,3-benzoxazol-2(3H)-ylidene(6-{[3-(3-oxo-4-morpholinyl)propyl]amino}-4-pyrimidinyl)ethanenitrile

1,3-benzoxazol-2(3H)-ylidene(5-methyl-2-{[3-(1H-1,2,4-triazol-1-yl)propyl]amino}-4-pyrimidinyl)ethanenitrile

1,3-benzoxazol-2(3H)-ylidene(5-methyl-2-{[3-(3-oxo-4-morpholinyl)propyl]amino}4-pyrimidinyl)ethanenitrile

1,3-benzoxazol-2(3H)-ylidene(2-{[3-(3-oxo-4-morpholinyl)propyl]amino}-4-pyrimidinyl)ethanenitrile

1,3-benzoxazol-2(3H)-ylidene(2-{[(2,2-dimethyl-4-oxo-4H-1,3-benzodioxin-6-yl)methyl]amino}-4-pyrimidinyl)ethanenitrile

methyl 5-[({4-[1,3-benzoxazol-2(3H)-ylidene(cyano)methyl]-2-pyrimidinyl}amino)methyl]-2-(2-methoxy-2-oxoethoxy)benzoate

N-[3-({4-[1,3-benzoxazol-2(3H)-ylidene(cyano)methyl]-2-pyrimidinyl}amino)propyl]-2-ethoxy-N-glycoloylacetamide

methyl 4-[2-({4-[1,3-benzoxazol-2(3H)-ylidene(cyano)methyl]-2-pyrimidinyl}amino)ethyl]benzoate

methyl 4-[({4-[1,3-benzoxazol-2(3H)-ylidene(cyano)methyl]-5-methyl-2-pyrimidinyl}amino)methyl]benzoate

methyl{4-[({4-[1,3-benzoxazol-2(3H)-ylidene(cyano)methyl]-2-pyrimidinyl}amino)methyl]phenoxy}acetate

methyl 5-[({4-[1,3-benzoxazol-2(3H)-ylidene(cyano)methyl]-2-pyrimidinyl}amino)methyl]-2-thiophenecarboxylate

1,3-benzoxazol-2(3H)-ylidene[2-({3-[4-(1-piperidinylsulfonyl)phenyl]propyl}amino)-4-pyrimidinyl]ethanenitrile

ethyl 4-[({4-[1,3-benzoxazol-2(3H)-ylidene(cyano)methyl]-2-pyrimidinyl}amino)methyl]-5-methyl-2-furoate

tert-butyl 4-[({4-[1,3-benzoxazol-2(3H)-ylidene(cyano)methyl]-5-methyl-2-pyrimidinyl}amino)methyl]-1-piperidinecarboxylate

1,3-benzoxazol-2(3H)-ylidene(2-{[3-(1-piperidinylsulfonyl)benzyl]amino}-4-pyrimidinyl)ethanenitrile

methyl 4-[2-({4-[1,3-benzoxazol-2(3H)-ylidene(cyano)methyl]-5-methyl-2-pyrimidinyl}amino)ethyl]benzoate

methyl 4-({4-[1,3-benzoxazol-2(3H)-ylidene(cyano)methyl]-2-pyrimidinyl}amino)butanoate

(2-amino-4-pyrimidinyl)(1,3-benzoxazol-2(3H)-ylidene)ethanenitrile

methyl 4-[({4-[1,3-benzoxazol-2(3H)-ylidene(cyano)methyl]-2-pyrimidinyl}amino)methyl]benzoate

tert-butyl 4-[({4-[1,3-benzoxazol-2(3H)-ylidene(cyano)methyl]-2-pyrimidinyl}amino)methyl]-1-piperidinecarboxylate

1,3-benzoxazol-2(3H)-ylidene{2-[(2-pyridin-2-ylethyl)amino]pyrimidin-4-yl}acetonitrile

1,3-benzoxazol-2(3H)-ylidene[2-(isopropylamino)pyrimidin-4-yl]acetonitrile

1,3-benzoxazol-2(3H)-ylidene{2-[(2,3-dimethylcyclohexyl)amino]pyrimidin-4-yl}acetonitrile

1,3-benzoxazol-2(3H)-ylidene{2-[(1-methylbutyl)amino]pyrimidin-4-yl}acetonitrile

1,3-benzoxazol-2(3H)-ylidene{2-[(pyridin-2-ylmethyl)amino]pyrimidin-4-yl}acetonitrile

1,3-benzoxazol-2(3H)-ylidene{2-[(3-butoxypropyl)amino]pyrimidinyl-4-yl}acetonitrile

1,3-benzoxazol-2(3H)-ylidene{2-[(pyridin-3-ylmethyl)amino]pyrimidin-4-yl}acetonitrile

1,3-benzoxazol-2(3H)-ylidene{2-[(3-isopropoxypropyl)amino]pyrimidin-4-yl}acetonitrile

1,3-benzoxazol-2(3H)-ylidene{2-[(1-ethylpropyl)amino]pyrimidin-4-yl}acetonitrile

1,3-benzoxazol-2(3H)-ylidene{2-[ethyl(isopropyl)amino]pyrimidin-4-yl}acetonitrile

1,3-benzoxazol-2(3H)-ylidene[2-(cyclopentylamino)pyrimidin-4-yl]acetonitrile

1,3-benzoxazol-2(3H)-ylidene[2-(cyclohexylamino)pyrimidin-4-yl]acetonitrile

1,3-benzoxazol-2(3H)-ylidene(6-methyl-2-{[3-(1H-1,2,4-triazol-1-yl)propyl]amino}pyrimidin-4-yl)acetonitrile

1,3-benzoxazol-2(3H)-ylidene[2-(cyclopentylamino)-6-methylpyrimidin-4-yl]acetonitrile

1,3-benzoxazol-2(3H)-ylidene[6-(4-ethylpiperazin-1-yl)pyrimidin-4-yl]acetonitrile

1,3-benzoxazol-2(3H)-ylidene[2-(cyclohexylamino)-6-methylpyrimidin-4-yl]acetonitrile

1,3-benzoxazol-2(3H)-ylidene{2-[benzyl(isopropyl)amino]pyrimidin-4-yl}acetonitrile

1,3-benzoxazol-2(3H)-ylidene[6-(cyclopentylamino)pyrimidin-4-yl]acetonitrile

1,3-benzoxazol-2(3H)-ylidene(2-{[4-(4-methyl-1-piperazinyl)-4-oxobutyl]amino}-4-pyrimidinyl)ethanenitrile

1,3-benzoxazol-2(3H)-ylidene(2-{[4-(4-morpholinyl)-4-oxobutyl]amino}-4-pyrimidinyl)ethanenitrile

1,3-benzoxazol-2(3H)-ylidene(2-{[4-oxo-4-(1-piperidinyl)butyl]amino}-4-pyrimidinyl)ethanenitrile

1,3-benzoxazol-2(3H)-ylidene[2-({4-[4-(2-methoxyethyl)-1-piperazinyl]-4-oxobutyl}amino)-4-pyrimidinyl]ethanenitrile

1,3-benzoxazol-2(3H)-ylidene(2-{[4-(1,4-dioxa-8-azaspiro[4.5]dec-8-yl)-4-oxobutyl]amino}-4-pyrimidinyl)ethanenitrile

1,3-benzoxazol-2(3H)-ylidene(2-{[4-oxo-4-(1-piperazinyl)butyl]amino}-4-pyrimidinyl)ethanenitrile

4-[({4-[1,3-benzoxazol-2(3H)-ylidene(cyano)methyl]-5-methyl-2-pyrimidinyl}amino)methyl]benzoic acid

4-[2-({4-[1,3-benzoxazol-2(3H)-ylidene(cyano)methyl]-2-pyrimidinyl}amino)ethyl]benzoic acid

4-[({4-[1,3-benzoxazol-2(3H)-ylidene(cyano)methyl]-2-pyrimidinyl}amino)methyl]benzoic acid

1,3-benzoxazol-2(3H)-ylidene[5-methyl-2-({4-[(4-methyl-1-piperazinyl)carbonyl]benzyl}amino)-4-pyrimidinyl]ethanenitrile

1,3-benzoxazol-2(3H)-ylidene{2-[(2-{4-[(4-methyl-1-piperazinyl)carbonyl]phenyl}ethyl)amino]-4-pyrimidinyl)ethanenitrile

4-[2-({4-[1,3-benzoxazol-2(3H)-ylidene(cyano)methyl]-2-pyrimidinyl}amino)ethyl]-N-[2-(dimethylamino)ethyl]benzamide

1,3-benzoxazol-2(3H)-ylidene[2-({4-[(4-methyl-1-piperazinyl)carbonyl]benzyl}amino-4-pyrimidinyl]ethanenitrile

1,3-benzoxazol-2(3H)-ylidene{5-methyl-2-[(4-piperidinylmethyl)amino]-4-pyrimidinyl}ethanenitrile

1,3-benzoxazol-2(3H)-ylidene{2-[(4-piperidinylmethyl)amino]-4-pyrimidinyl}ethanenitrile

(2-{[(1-acetyl-4-piperidinyl)methyl]amino}-4-pyrimidinyl)(1,3-benzoxazol-2(3H)-ylidene)ethanenitrile

1,3-benzoxazol-2(3H)-ylidene{2-[({1-[(dimethylamino)acetyl]-4-piperidinyl}methyl)amino]-4-pyrimidinyl}ethanenitrile

(2-{[(1-acetyl-4-piperidinyl)methyl]amino}-5-methyl-4-pyrimidinyl)(1,3-benzoxazol-2(3H)-ylidene)ethanenitrile

N-{4-[1,3-benzoxazol-2(3H)-ylidene(cyano)methyl]-2-pyrimidinyl}-4-(dimethylamino)butanamide

N-{4-[1,3-benzoxazol-2(3H)-ylidene(cyano)methyl]-2-pyrimidinyl}-1-methyl-4-piperidincarboxamide

Compounds of formula (I) are suitable for the use as medicament, in particular for the treatment and/or prevention of metabolic disorders mediated by insulin resistance or hyperglycemia, comprising diabetes type II, inadequate glucose tolerance, insulin resistance, obesity, polycystic ovary syndrome (PCOS).

The compounds according to formula I could be employed alone or in combination with further pharmaceutical agents.

A further aspect of the present invention is related to a pharmaceutical composition a comprising a benzothiazole derivative according to formula (I) and at least one further drug (in particular an anti-diabetes agent). In one embodiment the further diabetes agents are selected from the group comprising or consisting of insulin (or insulin mimicks), aldose reductase inhibitors, alpha-glucosidase inhibitors, sulfonyl urea agents, biguanides (e.g. metformin), thiazolidines (e.g. pioglitizone, rosiglitazone, cf. WO 02/100396), a PTP1B inhibitor, a PPAR agonists or a GSK-3 inhibitor.

Insulins useful with the method of the present invention include rapid acting insulins, intermediate acting insulins, long acting insulins and combination of intermediate and rapid acting insulins.

Aldose reductase inhibitors useful in the method of this invention include those known in the art.

Among the more preferred aldose reductase inhibitors of this invention are minalrestat, Tolrestat, Sorbinil, Methosorbinil, Zopolrestat, Epalrestat, Zenarestat, Imirestat and Ponalrestat or the pharmaceutically acceptable salt forms thereof.

The alpha-glucosidase inhibitors useful for the method of the present invention include miglitol or acarbose, or the pharmaceutically acceptable salt form thereof.

Sulfonylurea agents useful with the method of the present invention include glipizide, Glyburide (Glibenclamide), Clorpropamide, Tolbutamide, Tolazamide and Glimepiride, or the pharmaceutically acceptable salt forms thereof.

Preferably, said supplementary pharmaceutically active agent is selected from the group consisting of a rapid acting insulin, an intermediate acting insulin, a long acting insulin, a combination of intermediate and rapid acting insulins, Inalrestat, Tolrestat, Sorbinil, Methosorbinil, Zopolrestat, Epalrestat, Zenarestat, Imirestat, Ponalrestat, ONO-2235, GP-1447, CT-112, BAL-ARI 8, AD-5467, ZD5522, M-16209, NZ-314, M-79175, SPR-210, ADN 138, or SNK-860, Miglitol, Acarbose, Glipizide, Glyburide, Chlorpropamide, Tolbutamide, Tolazamide, or Glimepriride.

In one embodiment, the compounds of formula (I) are useful in inhibiting Glycogen Synthase Kinase 3

Still a further object of the present invention is a process for preparing the benzoxazole acetonitriles according to formula I.

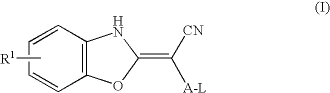

The benzoxazole acetonitriles exemplified in this invention may be prepared from readily available starting materials using the following general methods and procedures. It will be appreciated that where typical or preferred experimental conditions (i.e. reaction temperatures, time, moles of reagents, solvents, etc.) are given, other experimental conditions can also be used unless otherwise stated. Optimum reaction conditions may vary with the particular reactants or solvents used, but such conditions can be determined by one skilled in the art by routine optimisation procedures.

Generally, the benzoxazole acetonitrile derivatives according to the general formula I may be obtained by several processes using solution-phase chemistry protocols.

According to one process, benzoxazole acetonitrile derivatives according to the general formula I, whereby the substituents A, L and R¹ are as above defined, are prepared from the corresponding acetonitrile derivatives II and chloro derivatives III, by well known solution-phase chemistry protocols, such as those described in the Examples and shown in Scheme 1, below.

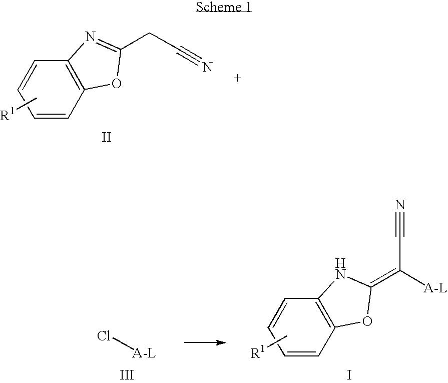

The chloro derivatives III may be obtained either from commercial sources or they may be prepared from known compounds using conventional procedures, known by one skilled in the art. Preferred chloro derivatives III are defined such as shown in the scheme 2 below.

More specifically, benzoxazole acetonitrile of general formula I may be prepared as follows: benzoxazole acetonitrile derivatives II, whereby R¹ is as above defined, is reacted with the bis-chloro derivatives III′, where A′ is as above defined, to give the intermediate of synthesis II′. In a subsequent step, the intermediate II′ is treated with the amines IV, whereby the substituents R³, R⁴ are as above defined to give the final benzoxazole acetonitrile derivatives I, utilizing well known solution-phase chemistry protocols, such as those described in the below Examples and illustrated in Scheme 2, below.

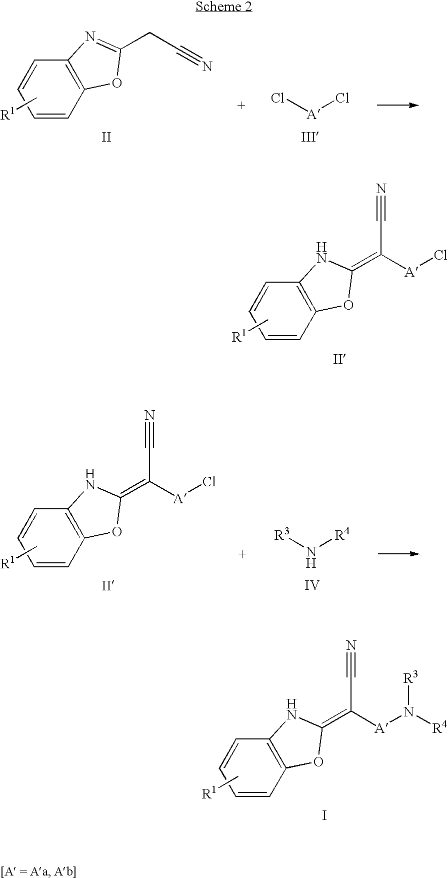

A′ is a pyrimidinyl core A′a and A′b as shown in the Scheme 3 below.

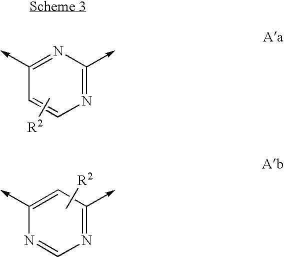

The benzoxazole acetonitrile derivatives according to the general formula Ia, whereby the substituent R¹ is as above defined, are obtained in two subsequent steps as illustrated in Scheme 4. In a first step, the chloro benzoxazole acetonitrile derivatives II′a are isolated after condensation of the benzoxazole acetonitrile compound II with bis-chloro derivative III′a, whereby the heteroaromatic core is A′a, and R² is as above defined. This first reaction step may be performed, using, e.g. lithium hydride or sodium hydride or similar reagents in an appropriate solvent such as THF or DMF. This reaction may be performed at various temperatures depending of the reactivity of compounds II and III′a, by traditional thermic method or using microwave technology, using standard conditions well known to the person skilled in the art (cf. the Examples below). In a subsequent step, chloro benzoxazole acetonitrile derivatives II′a are treated with various amines IV to give the benzoxazole acetonitrile derivatives Ia. The nucleophilic displacement of the chloro atom of the pyrimidinyl moiety by the amine IV, may be accomplished by treatment with several equivalents of the amines IV with the optional presence of sodium iodine as catalyst and a base such as triethylamine or diisopropylethylamine or similar reagents. This reaction may be performed at various temperatures depending of the intrinsic reactivity of compounds IV and II′a, by traditional thermic method or using microwave technology, using standard conditions well known to the person skilled in the art, such as those described hereinafter in the Examples.

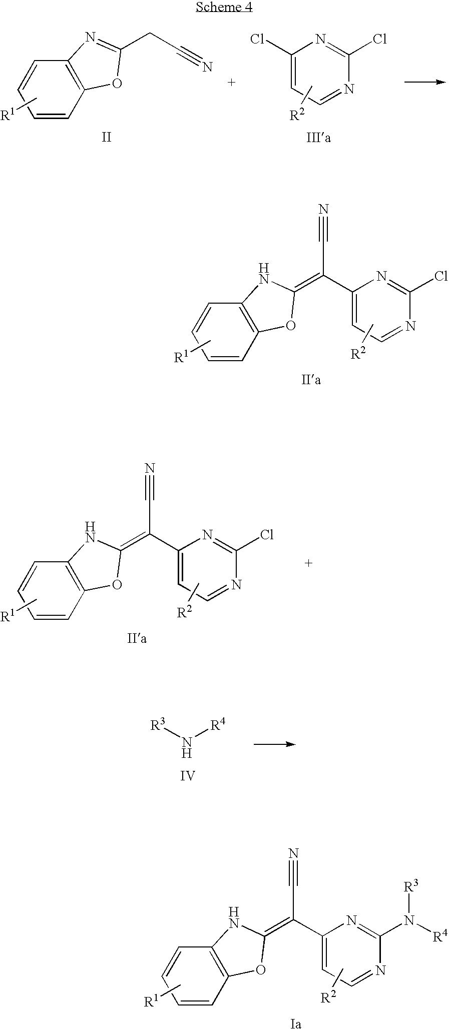

The benzoxazole acetonitrile derivatives according to the general formula Ib, whereby the substituent R¹ is as above defined, may be obtained in two subsequent steps as illustrated in the Scheme 5 below. In a first step, the benzoxazole acetonitrile derivatives II′b are isolated after condensation of the azole acetonitrile compound II with a bis-chloro derivative III′b, whereby the heteroaromatic core is A′b, and R² is as above defined. This first reaction step maybe performed, using, e.g. lithium hydride or sodium hydride or similar reagents in an appropriate solvent such as THF or DMF. This reaction may be performed at various temperatures depending of the reactivity of compounds II and III′b, by traditional thermic method or using microwave technology, using standard conditions well known to the person skilled in the art, such as those described hereinafter in the Examples. In a subsequent step, the chloro benzoxazole acetonitrile derivatives II′b are treated with various amines IV to give the expected benzoxazole acetonitriles derivatives Ib. The nucleophilic displacement of the chloro atom of the pyrimidinyl moiety by the amine IV, is accomplished by treatment with several equivalents of the amines IV with the optional presence of a catalyst like sodium iodine and a base such as triethylamine or diisopropylethylamine or similar reagents. This reaction may be performed at various temperatures depending of the reactivity of compounds IV and II′b, by traditional thermic method or using microwave technology, using standard conditions well known to the person skilled in the art, such as those described hereinafter in the Examples.

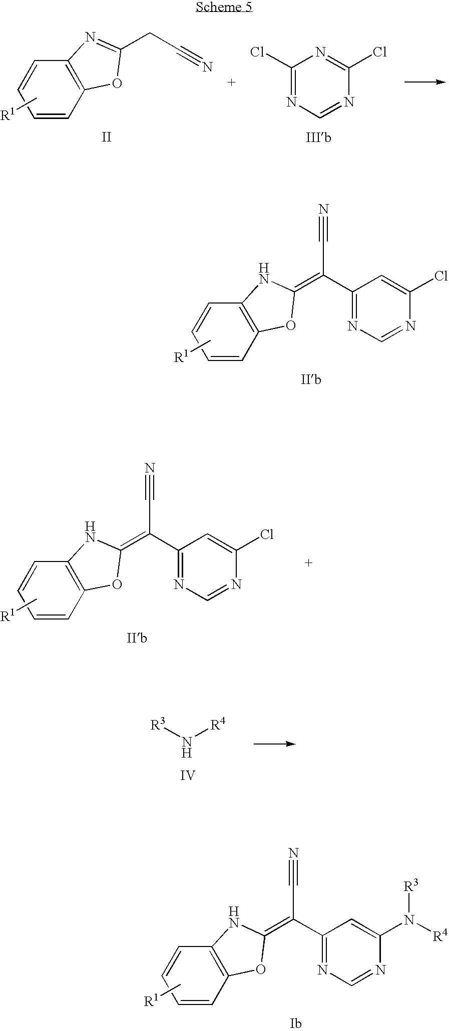

The benzoxazole acetonitrile derivatives according to the general formula Id, may be obtained in 2-6 subsequent steps depending the availability of starting materials and building blocks. In a first step, the benzoxazole acetonitrile derivatives Ic are isolated after condensation of the benzoxazole compound II′a with a solution of ammonium hydroxide, as shown in Scheme 6. This reaction may be performed in solvents like DMA, isopropanol or solution containing both solvents in various ratio and at various temperatures depending of the intrinsic reactivity of compounds II′a, by traditional thermal method or using microwave technology, using standard conditions well known to the person skilled in the art, such as those described hereinafter in the Examples.

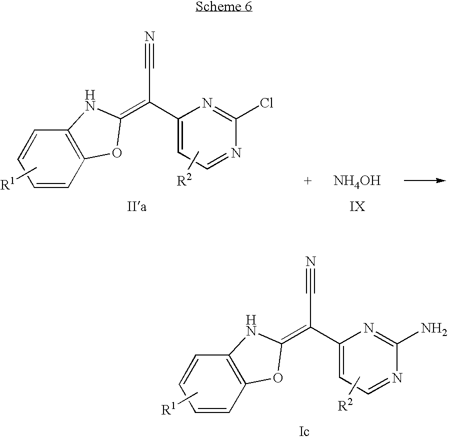

In a following step as shown in Scheme 7, the benzoxazole acetonitrile derivatives according to the general formula Id can be obtained from the intermediate Ic, whereby R³ is as above defined. The benzoxazole derivatives Id may be obtained by treatment of the intermediate Ic with either an acyl chloride or a carboxylic acid using standard conditions well known to the person skilled in the art, such as amide bond formation protocols using the appropriate reactants as those mentioned above and reagents such as bases like triethylamine, pyridine etc, and activating agents e.g. HOBt, EDC, Mukayama reagent or similar reagents in an appropriate solvent such as DCM, THF or DMF. This reaction can be performed at various temperatures depending of the intrinsic reactivity of compounds Ic and X, by traditional thermal method or using microwave technology, using standard conditions well known to the person skilled in the art, such as those described hereinafter in the Examples.

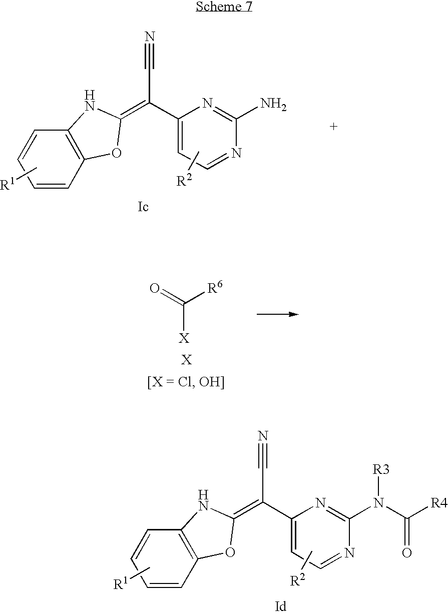

The benzoxazole acetonitrile components II are either obtained from commercial sources or prepared in two steps by conventional procedures from the condensation of the corresponding ortho hydroxyaniline derivatives VI and cyano acetic acid derivative VII followed by a cyclisation as outlined in scheme 8. The ortho hydroxyaniline derivatives VI and the cyano acetic acid derivative VII are either obtained from commercial sources or prepared by conventional procedures known by one skilled in the art.

Preferred intermediate compounds of formulae (II′a) or (II′b) are selected from the group consisting of:

1,3-benzoxazol-2(3H)-ylidene(2-chloro-6-methylpyrimidin-4-yl)acetonitrile

1,3-benzoxazol-2(3H)-ylidene(2-chloro-6-methylpyrimidin-4-yl)acetonitrile

1,3-benzoxazol-2(3H)-ylidene(6-chloropyrimidin-4-yl)acetonitrile

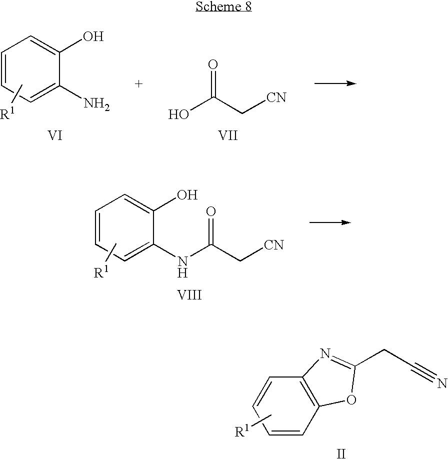

The dichloropyrimidinyl precursor compounds III′a and b maybe obtained from commercial sources.

If the above set out general synthetic methods are not applicable for the obtention of compounds of formula I, suitable methods of preparation known by a person skilled in the art should be used.

When employed as pharmaceuticals, the benzoxazole acetonitriles of the present invention are typically administered in the form of a pharmaceutical composition. Hence, pharmaceutical compositions comprising a compound of formula (I) and a pharmaceutically acceptable carrier, diluent or excipient therefore are also within the scope of the present invention. A person skilled in the art is aware of a whole variety of such carrier, diluent or excipient compounds suitable to formulate a pharmaceutical composition.

The compounds of the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous use). Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

When employed as pharmaceuticals, benzoxazole acetonitriles of this invention are typically administered in the form of a pharmaceutical composition. Such compositions can be prepared in a manner well known in the pharmaceutical art and comprise at least one active compound. Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of these inventions can be administered by a variety of routes including oral, rectal, transdermal, subcutaneous, intravenous, intramuscular, intrathecal, intraperitoneal and intranasal. Depending on the intended route of delivery, the compounds are preferably formulated as either injectable, topical or oral compositions. The compositions for oral administration may take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the benzoxazole acetonitrile compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As mentioned above, the benzoxazole acetonitriles of formula I in such compositions is typically a minor component, frequently ranging between 0.05 to 10% by weight with the remainder being the injectable carrier and the like.

The above described components for orally administered or injectable compositions are merely representative. Further materials as well as processing techniques and the like are set out in Part 5 of Remington's Pharmaceutical Sciences, 20^{th} Edition, 2000, Marck Publishing Company, Easton, Pa., which is incorporated herein be reference.

The compounds of this invention can also be administered in sustained release forms or from sustained release drug delivery systems. A description of representative sustained release materials can also be found in the incorporated materials in Remington's Pharmaceutical Sciences.

In the following the present invention shall be illustrated by means of some examples which are not construed to be viewed as limiting the scope of the invention.

The following abbreviations are hereinafter used in the accompanying examples: min (minute), hr (hour), g (gram), mmol (millimole), m.p. (melting point), eq (equivalents), mL (milliliter), μL (microliters), mL (milliliters), ACN (Acetonitrile), Boc (butoxycarbonyl), CDCl₃ (deuterated chloroform), CsCO₃ (Cesium carbonate), cHex (Cyclohexanes), DCM (Dichloromethane), DIC (Diisopropyl carbodiimide), DIPEA (Diisopropylamine), DMA (Dimethylacetamide), DMAP (4-Dimethylaminopyridine) DMF (Dimethylformamide), DMSO (Dimethyl-sulfoxide), DMSO-d₆ (deuterated dimethylsulfoxide), EDC (1-(3-Dimethyl-amino-propyl)-3-ethylcarbodiimide), Et₃N (Triethylamine), EtOAc (Ethyl acetate), EtOH (Ethanol), Et₂O (Diethyl ether), Fmoc (9-fluorenyl-methoxycarbonyl), HOBt (1-Hydroxybenzotriazole), iPrOH (Isopropanol), K₂CO₃ (potassium carbonate), LiH (Lithium Hydride), Mukayama reagent (1-methyl-2-chloropyridinium iodide), NaI (Sodium Iodine), NaH (Sodium hydride), NaHCO₃ (Sodium bicarbonate), NH₄Cl (Ammonium chloride), nBuLi (n Butyllithium), Pd(PPh₃)₄ (Palladium triphenylphosphine tetrakis), PTSA (p-toluene sulphonic acid), (TBTU (O-Benzotriazolyl-N,N,N′,N′-tetramethyluronium-tetrafluoroborate), TEA (Triethyl amine), TFA (Trifluoro-acetic acid), THF (Tetrahydrofuran), TMOF (trimethylorthoformate), MgSO₄ (Magnesium sulfate), PetEther (Petroleum ether), rt (room temperature).

The HPLC, NMR and MS data provided in the examples described below were obtained as follows: HPLC: column Waters Symmetry C8 50×4.6 mm, Conditions: MeCN/H₂O, 5 to 100% (8 min), max plot 230-400 nm; Mass spectra: PE-SCIEX API 150 EX (APCI and ESI), LC/MS spectra: Waters ZMD (ES); ¹H-NMR: Bruker DPX-300 MHz. The purifications were obtained as followed: Preparative HPLC Waters Prep LC 4000 System equipped with columns Prep Nova-Pak®HR C186 μm 60 Å, 40×30 mm (up to 100 mg) or 40×300 mm (up to 1 g). All the purifications were performed with a gradient of MeCN/H₂O 0.09% TFA.

### EXAMPLES

### Intermediate 1: 3-(1H-1,2,4-triazol-1-yl)propan-1-amine

### Step-1: 3-(1H-1,2,4-triazol-1-yl)propanenitrile

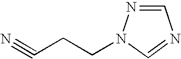

A mixture of 1,2,4-triazole (25 g, 0.362 mol) and acrylonitrile (100 mL, 4 w/v) was heated up to 80° C. under nitrogen for 16 h. The reaction mixture was then concentrated under reduced pressure to remove the excess of acrylonitrile affording 41 g of the title compound as a colorless liquid (93%). It was used in the next step without further purification.

### Step-2: 3-(1H-1,2,4-triazol-1-yl)propan-1-amine

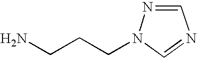

To a mixture of 3-(1H-1,2,4-triazol-1-yl)-propanenitrile (25 g, 0.204 mol) and Raney-Nickel (5 g, 0.2 w/w, wet) in methanol (300 mL) was added a solution of 25% aqueous NH₄OH (75 mL). The above reaction mixture was hydrogenated under pressure (75 psi of hydrogen) for a period of 6 h. The catalyst was then filtered off and the filtrate was concentrated under reduced pressure. The residue obtained was taken up in DCM (150 mL) then triturated 4 times and the combined organic layer was concentrated under reduced pressure to yield 22 g of the title compound as a liquid (85%). The above compound was converted to its hydrochloride using HCl gas in a mixture of ether/methanol (9.5/0.5) to yield 20 g of the product as its dihydrochloride.

¹H NMR (DMSO-d₆) δ 8.89 (s, 1H), 8.26 (s, 1H), 7.83 (s, 2H exchangeable), 4.33 (t, J=6.8 Hz, 2H), 2.85-2.74 (m, 2H), 2.13-2.03 (m, 2H).

### Intermediate 2: [2-(1H-1,2,4-triazol-1-yl)ethyl]amine

### Step-1 2-[2-(1H-1,2,4-triazol-1-yl)ethyl]-1H-isoindole-1,3(2H)-dione

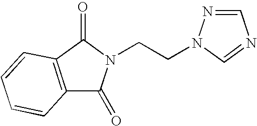

To a solution of of 1,2,4-triazole (50 g, 0.724 mol) in dry DMF (300 ml) at 0° C. was added sodium hydride (38 g, 0.797 mol, 50% ) in small portions over a period of 40 min and stirred for 2 h at ambient temperature. To the above reaction mixture was added a solution of 2-(bromoethyl)pthalimide (183 g, 0.724 mol) in DMF (200 ml) over a period of 45 min. The reaction mixture was heated to 60° C. under nitrogen for 16 h and cooled to room temperature. The reaction mixture was then diluted with excess of water and extracted with ethyl acetate (3×250 ml), washed with brine, dried and evaporated to a residue. The residue was purified by chromatography using pet ether/EtOAc (9/1 to 6/4) to yield 40 g (85%) of the title compound as a solid.

TLC-Pet/EtOAc (8/2), R_{f}=0.55

### Step-2 [2-(1H-1,2,4-triazol-1-yl)ethyl]amine.HCl

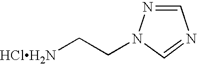

To a solution of 2-[2-(1H-1,2,4-triazol-1-yl)ethyl]-1H-isoindole-1,3(2H)dione (40 g, 0.165 mol) in ethanol (450 ml) at room temperature was added hydrazine hydrate (25 g, 0.495 mol) and the reaction mixture was heated to reflux for 10 h. The reaction mixture was cooled and the solid precipitated was filtered off. The filtrate was evaporated to a residue and purified by chromatography using chloroform/methanol (9/1 to 6/4) as eluent to afford 15 g of the free amine as a liquid. The free amine was converted into its hydrochloride by passing HCl gas in ethyl acetate to yield 15 g (63%) of the title compound as its dihydrochloride.

TLC-CHCl₃/MeOH (7/3), R_{f}=0.3 (free amine)

### Intermediate 3: 1-(2′-Aminopropyl)pyrazole

### Step-1: 3-(1H-pyrazol-1-yl)propanenitrile

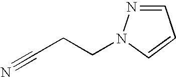

A mixture of pyrazole (25 g, 0.367 mol) and acrylonitrile (100 ml, 4 w/v) was heated to 80° C. under nitrogen for 20 h. The reaction mixture was then evaporated under reduced pressure to remove excess of acrylonitrile to give the title compound (40 g, 90%) as a colourless liquid.

TLC-CHCl₃/MeOH (8/2), R_{f}=0.5

### Step-2: 3: 1-(2′-Aminopropyl)pyrazole.HCl

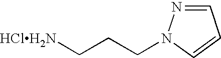

To a mixture of 3-(1H-pyrazol-1-yl)propanenitrile (25 g, 0.206 mol) and Raney-Nickel (5 g, 0.2 w/w, wet) in methanol (300 ml) was added 25% NH₄OH solution (75 ml, aqueous). The above reaction mixture was hydrogenated under a pressure of 75 psi of H₂ for a period of 8 h. The catalyst was then filtered off and the filtrate was evaporated to a residue under reduced pressure. The residue was triturated with CH₂Cl₂ (150 ml/4) and the combined organic layer was evaporated to yield the title compound (22 g, 85%) as a liquid. The above compound was converted to its hydrochloride by passing HCl gas in a mixture of EtOAc/methanol (9.5/0.5) to yield 20 g of the product as its dihydrochloride.

TLC-CHCl₃/MeOH(7/3), R_{f}=0.35 (Free amine)

### Intermediate 4: 1-(2′-Aminoethyl)pyrazole

### Step-1: 2-[2-(1H-pyrazol-1-yl)ethyl]-1H-isoindole-1,3(2H)-dione

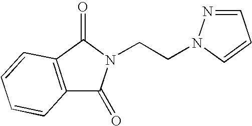

To a solution of pyrazole (25 g, 0.367 mol) in dry DMF (200 ml) at 0° C. was added sodium hydride (19 g, 0.404 mol, 50%) in small portions over a period of 30 min and stirred for 1 h at ambient temperature. To the above reaction mixture was added a solution of 2-(bromoethyl)pthalimide (93 g, 0.367 mol) in DMF (100 ml) over a period of 30 min. The reaction mixture was heated to 60° C. under nitrogen for 12 h and cooled to room temperature. The reaction mixture was then diluted with excess of water and extracted with ethyl acetate (3×250 ml), washed with brine, dried and evaporated to a residue. The residue was purified by chromatography using pet ether/EtOAc (9/1 to 7/3) to yield 14 g (60%) of the title compound as a solid.

TLC-Pet/EtOAc (8/2), R_{f}=0.6

### Step-2: 2-(1H-pyrazol-1-yl)ethanamine

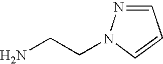

To a solution of 2-[2-(1H-pyrazol-1-yl)ethyl]-1H-isoindole-1,3(2H)-dione (13 g, 0.054 mol) in ethanol (150 ml) at room temperature was added hydrazine hydrate (5.5 g, 0.108 mol) and the reaction mixture was heated to reflux for 6 h. The reaction mixture was cooled and the solid precipitated was filtered off. The filtrate was evaporated to a residue and purified by chromatography using chloroform/methanol (9/1 to 6/4) as eluent to afford 5 g (78%) of the title compound as a liquid.

TLC-CHCl3/MeOH (7/3), R_{f}=0.3

### Intermediate 5: 1,3-benzoxazol-2-ylacetonitrile

### Step 1: 2-cyano-N-(2-hydroxyphenyl)acetamide

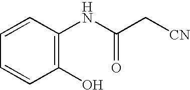

To a solution of cyanoacetic acid (17.1 g, 201 mmol) in dry DCM under N₂ at RT were added oxalyl chloride (26.7 g, 210 mmol) and 5 drops of dry DMF. The reaction started immediately to give off gas and it was allowed to stir at RT overnight. HPLC analysis showed only a small amount of unreacted hydroxyaniline. The reaction was quenched by adding 250 mL 1N HCl and stirred for 10 min. The solids were filtered and washed with 50 mL DCM, 50 mL H₂O, then 50 mL DCM and air dried for 2 hours, affording 22.8 g (71%) of 2-cyano-N-(2-hydroxyphenyl)acetamide. It was used in the next step without further purification.

### Step 2: 1,3-benzoxazol-2-ylacetonitrile

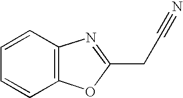

To a suspension of 2-cyano-N-(2-hydroxyphenyl)acetamide (22.50 g, 127 mmol) in 500 mL toluene under N2 was added PTSA (2.2 g, 12.7 mmol) and heated at reflux with a Dean-Stark flask for 5 hours. The reaction was cooled to 60° C. and filtered over a pad of basic alumina. The alumina was washed with 2×250 mL toluene and the filtrate concentrated to 50 mL. This was diluted with 100 mL of hexane and cooled to 0° C. It was then allowed to stand overnight. The solids were filtered and washed with 1×50 mL of 10% toluene in hexane to give 9.75 g (48%) of the title compound as a light brown solid.

LC (max plot): 99%, Rt: 1.82 min.

¹H-NMR (DMSO-d6): d: 7.85-7.70 (m, 2H), 7.52-7.35 (m, 2H), 4.69 (s, 2H)

### Intermediate 6: 4-(3-Aminopropyl)morpholin-3-one hydrochloride

### Step-1: 3-[(2-Hydroxyethyl)amino]propanenitrile

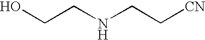

A mixture of ethanolamine (50 g, 0.819 mol) and acrylonitrile (43.4 g, 0.0819 mol) was heated up to 50° C. for 12 h. The reaction mixture was then evaporated under reduced pressure to give the title compound (94 g, 99%). It was used in the next step without further purification.

GC Purity->96%

TLC-CHCl₃/MeOH (8.5/1.5), R_{f}=0.3

### Step-2: 2-Chloro-N-(2-cyanoethyl)-N-(2-hydroxyethyl)acetamide

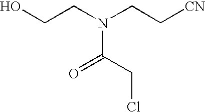

To a solution of 3-[(2-hydroxyethyl)amino]propanenitrile (50 g,0.435 mol) in dry dichloromethane (750 ml) at 0° C. was added dropwise chloroacetylchloride (59 g, 0.526 mol) over a period of 30 min under nitrogen. The reaction mixture was stirred at room temperature for 6 h and evaporated to near dryness. The residue was purified by chromatography using petrol ether/ethylacetate (8/2) as eluent to afford 50 g (60%) of the title compound as a liquid.

TLC-CHCl₃/MeOH (8.5/1.5), R_{f}=0.6

### Step-3: 3-(3-Oxomorpholin-4-yl)propanenitrile

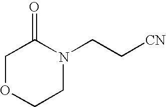

To a solution of 2-chloro-N-(2-cyanoethyl)-N-(2-hydroxyethyl)acetamide (40 g, 0.209 mol) in dry tert-butylalcohol (500 ml) at 0° C. under nitrogen was added potassium tert-butoxide (23.5 g, 0.2209 mol). The reaction mixture was refluxed for 12 h, cooled and evaporated to dryness under reduced pressure. The residue was diluted with cold-water (500 mL) and the product was extracted with ethylacetate (2×200 mL). The combined organic layer was washed with brine, dried and evaporated to near dryness. The residue was purified by chromatography using chloroform/methanol (9/1) as eluent to afford 25 g (78%) of the title compound as a solid.

TLC-CHCl₃/MeOH (8.5/1.5), R_{f}=0.8

### Step-4: 4-(3-Aminopropyl)morpholin-3-one hydrochloride

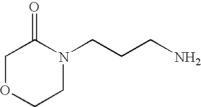

To a solution of 3-(3-oxomorpholin-4-yl)propanenitrile (25 g, 0.162 mol) in methanol (300 ml) were added ammonium hydroxide (75 ml, 25% aqueous solution) followed by Ra—Ni (5 g, wet) and the reaction mixture was hydrogenated under pressure (50 psi of hydrogen) for 8 h. The catalyst was then filtered off and the filtrate was concentrated under reduced pressure to afford the title compound. The above compound was converted to its hydrochloride by passing HCl gas in ether to yield 24 g (77%) of the title compound as a solid.

TLC-CHCl₃/MeOH (8.5/1.5), R_{f}=0.2 (free amine)

### Intermediate 7: 4-(3-Aminopropyl)morpholine-3,5-dione.HCl

### Step-1: Tert-Butyl-3-aminopropyl carbamate

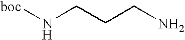

To a solution of 1,3-diaminopropane (100 g, 1.34 mol) in dry THF (1 L) at 0° C. was added Boc-anhydride (98 g, 0.45 mol). The reaction mixture was stirred at room temperature for 24 h under N₂. The reaction mixture was concentrated under reduced pressure. The residue was taken up in ethylacetate (2 L) and was washed with brine (3×250 mL) then dried and concentrated. The crude product was purified by chromatography (chloroform/methanol and methanol) to give tert-butyl-3-aminopropyl carbamate (65 g, 82%).

TLC, Chloroform/Methanol, 9.5:0.5, R_{f}=0.2

### Step-2: Tert-Butyl-3-(3,5-dioxomorpholin-4-yl)propylcarbamate

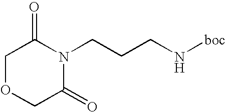

A mix of diglycolic anhydride (22 g, 0.188 mol), tert-butyl-3-aminopropylcarbamate (65 g, 0.377 mol) and N-methylmorpholine (21 mL, 0.188 mol) in dimethylacetamide (300 mL) was heated up to 120° C. for 48 h. The reaction mixture was cooled to room temperature. An excess of ethylacetate (1.5 L) was added and was washed with brine (5×150 mL) then dried and concentrated under reduced pressure. The crude was purified by chromatography (15% ethylacetate in chloroform) to give the title compound (15 g, 30%).

TLC, Chloroform/Methanol, 9:1, R_{f}=0.8

### Step-3: 4-(3-Aminopropyl)morpholine-3,5-dione.HCl

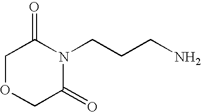

To a solution of tert-butyl-3-(3,5-dioxomorpholin-4-yl)propylcarbamate (15 g) in dry ether (150 mL) was added a saturated solution of diethyl ether (300 mL) with dry HCl (g) at 0° C. The reaction mixture was slowly allowed to warm up to room temperature. The precipitate obtained was filtered off and washed with cold ether then dried under vacuum to afford the title compound (11 g, 94%).

TLC, Chloroform/Methanol, 9:1, R_{f}=0.05

Procedure A

### Example 1

### 1,3-benzoxazol-2(3H)-ylidene(2-chloro-6-methylpyrimidin-4-yl)acetonitrile

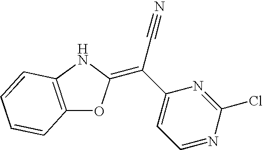

To a suspension sodium hydride (8.27 g; 0.19 mol) in THF (300.00 ml) was added dropwise a solution of 1,3-benzoxazol-2-ylacetonitrile (10 g, 0.063 mol) in THF (300.00 ml) at 0° C. The mixture was stirred at 0° C. for 1 h. Then the 2,4-dichloropyrimidine (10.36 g, 0.07 mol) was added portionwise and the reaction was stirred at rt overnight. The reaction was quenched by addition of water (100 ml) at 0° C. and the solution was evaporated. The THF was evaporated and the resulting aqueous phase was acidified with HCl 5N. After 3 h at 4° C., the solid was filtered off and washed with water until neutral pH and then with pentane to remove the oil. The red solid was dried under vacuum at 40° C. to afford 16 g (97%) of the title compound.

HPLC (max plot) 75%, Rt=3.33 min.

¹H-NMR (MeOD): d: 7.80-7.67 (m, 2H), 7.40-7.22 (m, 2H), 7.13-6.92 (m, 2H)

### Example 2

### 1,3-benzoxazol-2(3H)-ylidene(2-chloro-6-methylpyrimidin-4-yl)acetonitrile

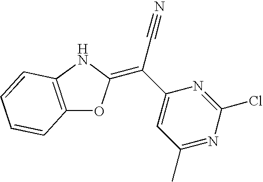

Following the general strategies and protocols outlined in the procedure A, the title compound was obtained from 1,3-benzoxazol-2-ylacetonitrile and 6-methyl-2,4-dichloropyrimidine in the presence of NaH in THF (94%).

M⁺(ES): 285.22; LC (215 nm): 71%, Rt: 1.41 min

### Example 3

### 1,3-benzoxazol-2(3H)-ylidene(6-chloropyrimidin-4-yl)acetonitrile

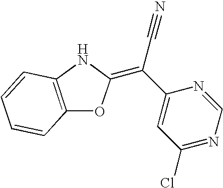

Following the general strategies and protocols outlined in the procedure A, the title compound was obtained from 1,3-benzoxazol-2-ylacetonitrile and 6,4-dichloropyrimidine in the presence of NaH in THF (98%).

¹H NMR (DMSO-d₆) δ 13.46 (br s, 1H exchangeable), 8.72 (s,1H), 7.70 (d, J=7.5 Hz, 1H), 7.60 (d, J=7.6 Hz, 1H), 7.39-7.29 (m, 2H), 7.18 (br s, 1H)

M⁻(ES): 271.2; M⁺(ES): 269.2; HPLC (max plot) 99.83%; Rt: 3.50 min.

### Example 4

### 1,3-benzoxazol-2(3H)-ylidene(2-chloro-5-metylpyrimidin-4-yl)acetonitrile

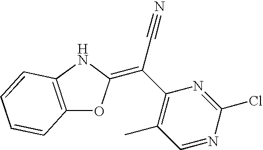

Following the general strategies and protocols outlined in the procedure A, the title compound was obtained from 1,3-benzoxazol-2-ylacetonitrile and 5-methyl-2,4-dichloropyrimidine in the presence of NaH in THF (99%).

¹H NMR (DMSO-d₆) δ 12.68 (br s, 1H exchangeable), 8.26 (s,1H), 7.69 (d, J=7.9 Hz, 1H), 7.61 (d, J=7.5 Hz, 1H), 7.40-7.29 (m, 2H), 2.41 (s, 3H)

M⁻(ES): 283.1; M⁺(ES): 285.2; HPLC (max plot) 96.41%; Rt: 3.46 min.

Procedure B

### Example 5

### 1,3-benzoxazol-2(3H)-ylidene(2-{[3-(2-oxopyrrolidin-1-yl)propyl]amino}pyrimidin-4-yl)acetonitrile

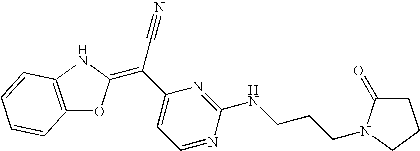

To a solution of 1,3-benzoxazol-2(3H)-ylidene(2-chloro-6-methylpyrimidin-4-yl)acetonitrile (243 mg, 0.81 mmol) in EtOH were added the amine (0.23 ml, 1.62 mmol) and triethylamine (0.375 ml, 1.62 mmol) and the solution was heated up to 155° C. in the microwave on high absorption for 4 mins. Analysis showed the reaction was complete. The yellow precipitate formed was filtered off and washed with water (×3) then dried under vacuum at 40° C.

The solid was taken up in DCM to which TFA was added. Ether in excess was added and the precipitate obtained was filtered off and washed with ether (3×) then dried under vacuum at 40° C. overnight, affording 240 mg (60%) of the title compound as a yellow powder.

HPLC (max plot) 99.8%, rt=2.46 min., LCMS (ES+): 377.26,

¹H-NMR (DMSO) 7.93-7.23 (m, 6H), 3.38-3.27 (m, 6H), 2.23-2.18 (m, 2H), 1.94-1.77 (m, 4H).

### Example 6

### 1,3-benzoxazol-2(3H)-ylidene(2-{[3-(1H-pyrazol-1-yl)propyl]amino}-pyrimidin-4-yl)acetonitrile

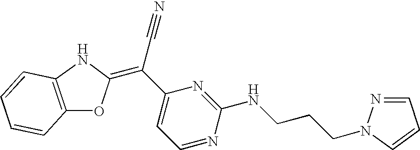

Following the general strategies and protocols outlined in the procedure B, the title compound was obtained from 1,3-benzoxazol-2(3H)-ylidene(2-chloro-6-methylpyrimidin-4-yl)acetonitrile and [3-(1H-pyrazol-1yl)propyl]amine in the presence of triethylamine for 5 min at 155° C. in EtOH (74%).

¹H NMR (DMSO-d₆) δ 12.87-8.62 (brs, 1H), 8.00-6.20 (m, 8H), 6.14 (s, 1H), 4.20-4.00 (m, 2H), 3.50-3.10 (m, 2H), 2.25-2.1.80 (m, 2H).

M⁻(ES): 358.37; M⁺(ES): 360.35; HPLC (max plot) 98%; Rt: 2.65 min.

### Example 7

### 1,3-benzoxazol-2(3H)-ylidene(2-{[2-(1H-1,2,4-triazol-1-yl)ethyl]amino}-pyrimidin-4-yl)acetonitrile

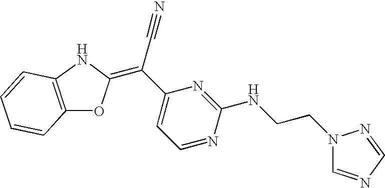

Following the general strategies and protocols outlined in the procedure B, the title compound was obtained from 1,3-benzoxazol-2(3H)-ylidene(2-chloro-6-methylpyrimidin-4-yl)acetonitrile and [2-(1H-1,2,4-triazol-1yl)ethyl]amine in the presence of triethylamine for 5 min at 155° C. in EtOH (74%). ¹H NMR (DMSO-d₆) δ 12.98-11.18 (br s, 1H), 8.53 (s, 1H), 8.15-7.00 (m, 8H), 6.41 (d, J=5.5 Hz, 0.4H), 4.53-4.42 (m, 2H), 3.85-3.68 (m, 2H).

M⁻(ES): 345.34; M⁺(ES): 347.34; HPLC (max plot) 98%; Rt: 2.14 min.

### Example 8

### 1,3-benzoxazol-2(3H)-ylidene(2-{[2-(1H-pyrazol-1-yl)ethyl]amino}pyrimidin-4-yl)acetonitrile

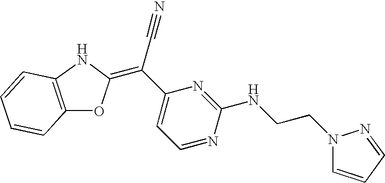

Following the general strategies and protocols outlined in the procedure B, the title compound was obtained from 1,3-benzoxazol-2(3H)-ylidene(2-chloro-6-methylpyrimidin-4-yl)acetonitrile and [2-(1H-pyrazol-1yl)ethyl]amine in the presence of triethylamine for 5 min at 155° C. in EtOH (74%). ¹H NMR (DMSO-d₆) δ 13.01-11.22 (brs, 1H), 8.00-6.20 (m, 9H), 4.60-4.20 (m, 2H), 4.00-3.60 (m, 2H).

M⁻(ES): 344.39; M⁺(ES): 346.36; HPLC (max plot) 98%; Rt: 2.58 min.

### Example 9

### 1,3-benzoxazol-2(3H)-ylidene{2-[(2-pyridin-3-ylethyl)amino]pyrimidin-4-yl}acetonitrile

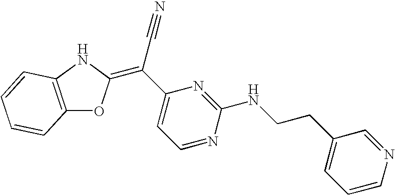

Following the general strategies and protocols outlined in the procedure B, the title compound was obtained from 1,3-benzoxazol-2(3H)-ylidene(2-chloro-6-methylpyrimidin-4-yl)acetonitrile and 3-(2-aminoethyl)pyridine in the presence of triethylamine for 5 min at 155° C. in EtOH (51%).

¹H NMR (DMSO-d₆) δ 11.28 (br s, 1H), 8.79 (m, 1H), 8.71-8.69 (m, 1H), 8.38-8.30 (m, 1H), 7.84-7.73 (m, 2H), 7.67-7.47 (m, 3H), 7.34-7.14 (m, 2H), 3.72-3.63 (m, 2H), 3.11-3.07 (m, 2H).

M⁻(ES): 355.26; M⁺(ES): 357.26; HPLC (max plot) 99.7%; Rt: 1.93 min.

### Example 10

### 1,3-benzoxazol-2(3H)-ylidene[2-(cyclopropylamino)pyrimidin-4-yl]acetonitrile

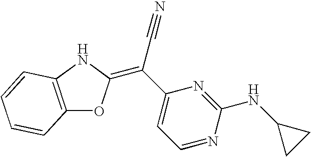

Following the general strategies and protocols outlined in the procedure B, the title compound was obtained from 1,3-benzoxazol-2(3H)-ylidene(2-chloro-6-methylpyrimidin-4-yl)acetonitrile and cyclopropylamine in the presence of triethylamine for 5 min at 155° C. in EtOH (74%).

¹H NMR (DMSO-d₆) δ 13.89-11.2 (br s, 1H), 8.90 (s, 1H), 8.50-7.0 (m, 8H), 6.38 (d, J=5.3 Hz, 1H), 3.00-2.75 (m, 1H), 1.23-0.60 (m, 4H).

M⁻(ES): 290.34; M⁺(ES): 292.37; HPLC (max plot) 99%; Rt: 2.53 min.

### Example 11

### 1,3-benzoxazol-2(3H)-ylidene(2-{[3-(1H-1,2,4-triazol-1-yl)propyl]amino}pyrimidin-4-yl)acetonitrile

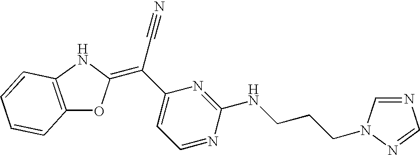

Following the general strategies and protocols outlined in the procedure B, the title compound was obtained from 1,3-benzoxazol-2(3H)-ylidene(2-chloro-6-methylpyrimidin-4-yl)acetonitrile and [3-(1H-1,2,4-triazol-1yl)propyl]amine in the presence of triethylamine for 5 min at 155° C. in EtOH (74%).

¹H NMR (DMSO-d₆) δ 13.01-11.17 (br s, 1H), 8.80-620 (m, 8H), 4.80-4.00 (m, 2H), 3.60-3.20 (m, 2H), 2.30-2.00 (m, 2H).

M⁻(ES): 359.37; M⁺(ES): 361.33; HPLC (max plot) 98%; Rt: 2.22 min.

### Example 12

### 1,3-benzoxazol-2(3H)-ylidene(6-{[3-(3-oxo-4-morpholinyl)propyl]amino}-4-pyrimidinyl)ethanenitrile

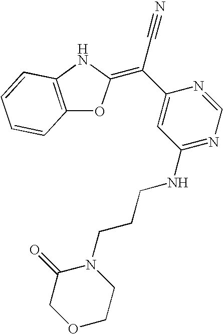

Following the general strategies and protocols outlined in the procedure B, the title compound was obtained from 1,3-benzoxazol-2(3H)-ylidene(6-chloropyrimidin-4-yl)acetonitrile and 4-(3-aminopropyl)morpholin-3-one.HCl in the presence of triethylamine for 20 min at 155° C. in EtOH (13%).

¹H NMR (DMSO-d₆) δ 13.37-13.27 (m, 1H), 8.56-8.44 (m, 1H), 8.24-8.11 (m, 1H), 7.56-7.43 (m, 2H), 7.26-7.12 (m, 2H), 6.14-5.74 (m, 1H), 4.01 (s, 2H), 3.85-3.78 (m, 2H), 3.45-3.27 (m, 6H), 1.90-1.75 (m, 2H)

M⁻(ES): 391.2; M⁺(ES): 393.2; HPLC (max plot) 93.59%; Rt: 2.80 min.

### Example 13

### 1,3-benzoxazol-2(3H)-ylidene(5-methyl-2-{[3-(1H-1,2,4-triazol-1-yl)propyl]amino}-4-pyrimidinyl)ethanenitrile

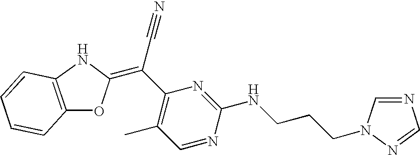

Following the general strategies and protocols outlined in the procedure B, the title compound was obtained from 1,3-benzoxazol-2(3H)-ylidene(2-chloro-5-metylpyrimidin-4-yl)acetonitrile and 3-(1H-1,2,4-triazol-1-yl)propan-1-amine.HCl in the presence of triethylamine for 10 min at 155° C. in EtOH/iPrOH 1:1 (48%).

¹H NMR (DMSO-d₆) δ 13.91 (br s, 1H exchangeable), 8.57 (s, 1H), 8.47 (br s, 1H exchangeable), 7.99 (s, 1H), 7.76 (s, 1H), 7.68 (d, J=7.9 Hz, 1H), 7.59 (d, J=7.2 Hz, 1H), 7.39-7.26 (m, 2H), 4.30 (t, J=6.8 Hz, 2H), 3.38-3.37 (m, 2H), 2.34 (s, 3H), 2.15 (quint., J=6.8 Hz, 2H)

M⁻(ES): 373.3; M⁺(ES): 375.3; HPLC (max plot) 93.3%; Rt: 2.33 min.

### Example 14

### 1,3-benzoxazol-2(3H)-ylidene(5-methyl-2-{[3-(3-oxo-4-morpholinyl)propyl]amino}-4-pyrimidinyl)ethanenitrile

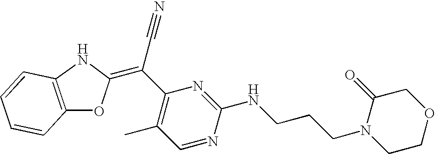

Following the general strategies and protocols outlined in the procedure B, the title compound was obtained from 1,3-benzoxazol-2(3H)-ylidene(2-chloro-5-metylpyrimidin-4-yl)acetonitrile and 4-(3-aminopropyl)morpholin-3-one.HCl in the presence of triethylamine for 10 min at 155° C. in EtOH/iPrOH 1:1 (37%).

¹H NMR (DMSO-d₆) δ 13.85 (br s, 1H), 8.39 (br s, 1H), 7.76 (s, 1H), 7.68 (d, J=7.6 Hz, 2H), 7.62 (d, J=7.2 Hz, 2H), 4 (s, 2H), 3.80 (m, 2H), 3.46-3.33 (m, 6H), 2.34 (s, 3H), 1.86 (m, 2H)

M⁻(ES): 405.3; M⁺(ES): 407.3; HPLC (max plot) 98.6%; Rt: 2.44 min.

### Example 15

### 1,3-benzoxazol-2(3H)-ylidene(2-{[3-(3-oxo-4-morpholinyl)propyl]amino}-4-pyrimidinyl)ethanenitrile

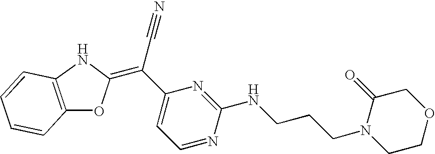

Following the general strategies and protocols outlined in the procedure B, the title compound was obtained from 1,3-benzoxazol-2(3H)-ylidene(2-chloro-4-pyrimidinyl)ethanenitrile and 4-(3-aminopropyl)morpholin-3-one.HCl in the presence of triethylamine for 15 min at 155° C. in EtOH (14%).

¹H NMR (DMSO-d₆) δ 11.40-11.10 (s, 1H), 8.80-6.20 (m, 7H), 4.02 (s, 2H), 3.90-3.75 (m, 2H), 3.55-3.25 (m, 6H), 1.95-1.75 (m, 2H)

M⁻(ES): 391; M⁺(ES): 393; HPLC (max plot) 91.47%; Rt: 2.33 min.

### Example 16

### 1,3-benzoxazol-2(3H)-ylidene(2-{[(2,2-dimethyl-4-oxo-4H-1,3-benzodioxin-6-yl)methyl]amino}-4-pyrimidinyl)ethanenitrile

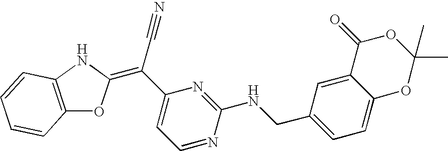

Following the general strategies and protocols outlined in the procedure B, the title compound was obtained from 1,3-benzoxazol-2(3H)-ylidene(2-chloro-4-pyrimidinyl)ethanenitrile and 6-(aminomethyl)-2,2-dimethyl-4H-1,3-benzodioxin.Acetate in the presence of triethylamine for 6 min at 155° C. in EtOH (45%).

¹H NMR (DMSO-d₆) δ 11.40-11.20 (br s, 1H), 9.20-6.20 (m, 10H), 4.70-4.50 (m, 2H), 1.67 (s, 6H)

M⁻(ES): 440.3; M⁺(ES): 442.3; HPLC (max plot) 89.60%; Rt: 3.22 min.

### Example 17

### methyl 5-[({4-[1,3-benzoxazol-2(3H)-ylidene(cyano)methyl]-2-pyrimidinyl}amino)methyl]-2-(2-methoxy-2-oxoethoxy)benzoate

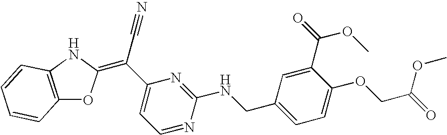

Following the general strategies and protocols outlined in the procedure B, the title compound was obtained from 1,3-benzoxazol-2(3H)-ylidene(2-chloro-4-pyrimidinyl)ethanenitrile and methyl 5-(aminomethyl)-2-(2-methoxy-2-oxoethoxy)benzoate.Acetate in the presence of triethylamine for 6 min at 155° C. in MeOH (21%).

¹H NMR (DMSO-d₆) δ 11.30-11-10 (br s, 1H), 9.20-6.20 (m, 10H), 4.86 (s, 2H), 4.65-4.45 (m, 2H), 3.78 (s, 3H), 3.67 (s, 3H)

M⁻(ES): 486.3; M⁺(ES): 488.4; HPLC (max plot) 98.62%; Rt: 2.98 min.

### Example 18

### N-[3-({4-[1,3-benzoxazol-2(3H)-ylidene(cyano)methyl]-2-pyrimidinyl}amino)propyl]-2-ethoxy-N-glycoloylacetamide

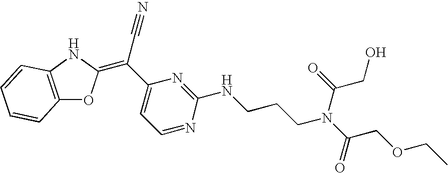

Following the general strategies and protocols outlined in the procedure B, the title compound was obtained from 1,3-benzoxazol-2(3H)-ylidene(2-chloro-4-pyrimidinyl)-ethanenitrile and 4-(3-aminopropyl)morpholin-3,5-dione in the presence of triethylamine for 16 min at 155° C. in EtOH (10%).

¹H NMR (DMSO-d₆) δ 11.40-11.00 (br s, 1H), 8.90-6.02 (m, 7H), 4.18 (s, 2H), 4.10 (q, J=7.2 Hz, 2H), 3.96 (s, 2H), 3.60-310 (m, 4H), 1.90-1-60 (m, 3H), 1.18 (t, J=7.2 Hz Hz, 3H)

M⁻(ES): 451.4; M⁺(ES): 453.5; HPLC (max plot) 97.10%; Rt: 2.63 min.

### Example 19

### methyl 4-[2-({4-[1,3-benzoxazol-2(3H)-ylidene(cyano)methyl]-2-pyrimidinyl}amino)ethyl]benzoate

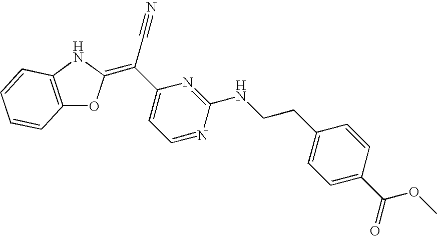

Following the general strategies and protocols outlined in the procedure B, the title compound was obtained from 1,3-benzoxazol-2(3H)-ylidene(2-chloro-4-pyrimidinyl)ethanenitrile and methyl-4-(2-aminoethyl)benzoate.HCl in the presence of triethylamine for 5 min at 155° C. in MeOH (20%).

¹H NMR (DMSO-d₆) δ 11.30.11.00 (br s, 1H), 9.10-6.2 (m, 11H), 3.82 (s, 3H), 3.70-3.50 (m, 2H), 3.15-2.90 (m, 2H)

M⁻(ES): 412; M⁺(ES): 414; HPLC (max plot) 91.52%; Rt: 3.20 min.

### Example 20

### methyl 4-[({4-[1,3-benzoxazol-2(3H)-ylidene(cyano)methyl]-5-methyl-2-pyrimidinyl}amino)methyl]benzoate

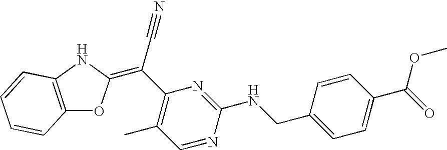

Following the general strategies and protocols outlined in the procedure B, the title compound was obtained from 1,3-benzoxazol-2(3H)-ylidene(2-chloro-5-metylpyrimidin-4-yl)acetonitrile and methyl-4-(aminomethyl)benzoate.HCl in the presence of triethylamine for 5 min at 155° C. in MeOH (75%).

¹H NMR (DMSO-d₆) δ: 8.98 (s exchangeable, 1H), 7.94 (d, J=8.3 Hz, 2H), 7.76 (s, 1H), 7.67 (d, J=7.9 Hz, 1H), 7.56-7.50 (m, 3H), 7.38-7.25 (m, 2H), 4.71-4.70 (br d, 2H), 3.82 (s, 3H), 2.34 (s, 3H)

M⁻(ES): 412.1; M⁺(ES): 414.1; HPLC (max plot) 94.89%; Rt: 3.29 min.

### Example 21

### methyl{4-[({4-[1,3-benzoxazol-2(3H)-ylidene(cyano)methyl]-2-pyrimidinyl}amino)methyl]phenoxy}acetate

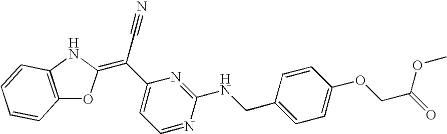

Following the general strategies and protocols outlined in the procedure B, the title compound was obtained from 1,3-benzoxazol-2(3H)-ylidene(2-chloro-4-pyrimidinyl)ethanenitrile and methyl-[4-(aminomethyl)phenoxy]Acetate.Acetate in the presence of triethylamine for 10 min at 155° C. in MeOH (31%).

¹H NMR (DMSO-d₆) δ 11.10-11.05 (br s, 1H), 9.20-6.20 (m, 11H), 4.77 (s, 2H), 4.60-4.40 (m, 2H), 3.68 (s, 3H)

M⁻(ES): 428.2; M⁺(ES): 430.2; HPLC (max plot) 82.00%; Rt: 3.02 min.

### Example 22

### methyl 5-[({4-[1,3-benzoxazol-2(3H)-ylidene(cyano)methyl]-2-pyrimidinyl}amino)methyl]-2-thiophenecarboxylate

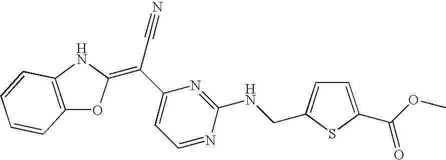

Following the general strategies and protocols outlined in the procedure B, the title compound was obtained from 1,3-benzoxazol-2(3H)-ylidene(2-chloro-4-pyrimidinyl)ethanenitrile and methyl-5-(aminoethyl)thiophene-2-carboxylate.HCl in the presence of triethylamine for 10 min at 155° C. in MeOH (39%).

¹H NMR (DMSO-d₆) δ: 11.50-11.40 (br s, 1H), 9.50-6.20 (m, 9H), 4.95-4.83 (m, 2H), 3.87 (s, 3H)

M⁻(ES): 404.1; M⁺(ES): 406.1; HPLC (max plot) 96.91%; Rt: 3.07 min.

### Example 23

### 1,3-benzoxazol-2(3H)-ylidene[2-({3-[4-(1-piperidinylsulfonyl)phenyl]-propyl}amino)-4-pyrimidinyl]ethanenitrile

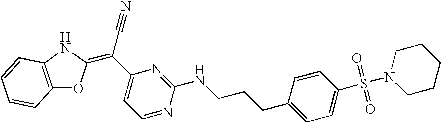

Following the general strategies and protocols outlined in the procedure B, the title compound was obtained from 1,3-benzoxazol-2(3H)-ylidene(2-chloro-4-pyrimidinyl)ethanenitrile and 3-[4-piperidine-1-sulfonyl)-phenyl]-propylamine.HCl in the presence of triethylamine for 5 min at 155° C. in EtOH (55%).

¹H NMR (DMSO-d₆) δ 11.10-11.06 (br s, 1H), 9.00-6.20 (m, 11H), 3.50-3.25 (m, 2H), 2.90-2.70 (m, 6H), 2.05-1.85 (m, 2H), 1.60-1.45 (m, 4H), 1.40-1.25 (m, 2H)

M⁻(ES): 515.2; M⁺(ES): 517.2; HPLC (max plot) 97.68%; Rt: 3.59 min.

### Example 24

### ethyl 4-[({4-[1,3-benzoxazol-2(3H)-ylidene(cyano)methyl]-2-pyrimidinyl}amino)methyl]-5-methyl-2-furoate

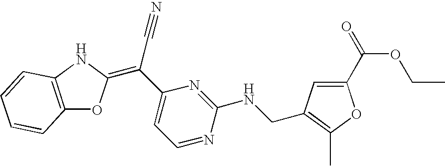

Following the general strategies and protocols outlined in the procedure B, the title compound was obtained from 1,3-benzoxazol-2(3H)-ylidene(2-chloro-4-pyrimidinyl)ethanenitrile and 4-aminomethyl-5-methylfuran-2-carboxylic acid ethyl ester in the presence of triethylamine for 10 min at 155° C. in EtOH (27%).

¹H NMR (DMSO-d₆) δ 11.20-11.10 (br s, 1H), 9.20-6.20 (m, 8H), 4.50-4.30 (m, 2H), 4.22 (qJ=6.8 Hz; J=7.1 Hz, 2H), 2.41 (s, 3H), 1.24 (t, J=J=6.8 Hz, J=7.1 Hz, 3H)

M⁻(ES): 416.2; M⁺(ES): 418.1; HPLC (max plot) 97.23%; Rt: 3.21 min.

### Example 25

### tert-butyl 4-[({4-[1,3-benzoxazol-2(3H)-ylidene(cyano)methyl]-5-methyl-2-pyrimidinyl}amino)methyl]-1-piperidinecarboxylate

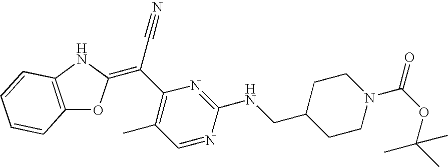

Following the general strategies and protocols outlined in the procedure B, the title compound was obtained from 1,3-benzoxazol-2(3H)-ylidene(2-chloro-5-metylpyrimidin-4-yl)acetonitrile (100.00 mg; 0.35 mmol) and 4-(aminomethyl)1-N-boc-piperidine (150.90 mg; 0.70 mmol) in the presence of triethylamine for 10 min at 155° C. in MeOH (84%).

HPLC (max plot) 3.51%; Rt: 3.51 min.

### Example 26

### 1,3-benzoxazol-2(3H)-ylidene(2-{[3-(1-piperidinylsulfonyl)benzyl]amino}-4-pyrimidinyl)ethanenitrile

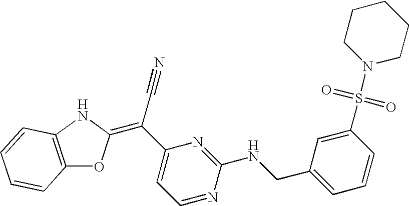

Following the general strategies and protocols outlined in the procedure B, the title compound was obtained from 1,3-benzoxazol-2(3H)-ylidene(2-chloro-4-pyrimidinyl)ethanenitrile and 3-(piperidine-1-sulfonyl)-benzylamine.HCl in the presence of triethylamine for 10 min at 155° C. in EtOH (57%).

¹H NMR (DMSO-d₆) δ 11.50-11.20 (br s, 1H), 9.20-6.20 (m, 11H), 4.90-4.50 (m, 2H), 2.90-2.60 (m, 4H), 1.60-1.00 (m, 6H)

M⁻(ES): 487.1; M⁺(ES): 489.2; HPLC (max plot) 91.09%; Rt: 3.42 min.

### Example 27

### methyl 4-[2-({4-[1,3-benzoxazol-2(3H)-ylidene(cyano)methyl]-5-methyl-2-pyrimidinyl}amino)ethyl]benzoate

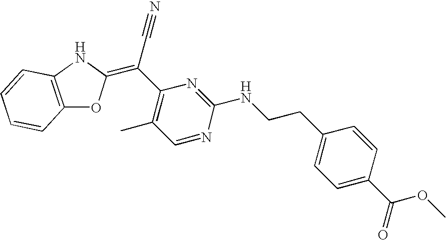

Following the general strategies and protocols outlined in the procedure B, the title compound was obtained from 1,3-benzoxazol-2(3H)-ylidene(2-chloro-5-metylpyrimidin-4-yl)acetonitrile and methyl-4-(2-aminoethyl)benzoate.HCl in the presence of triethylamine for 5 min at 155° C. in MeOH (33%).

¹H NMR (DMSO-d₆) δ 13.99 (s, 1H), 8.46 (br t, 1H), 7.88 (d, J=7.9 Hz, 2H), 7.78 (s, 1H), 7.67-7.64 (m, 1H), 7.44-7.41 (m, 3H), 7.33-7.23 (m, 2H), 3.83 (s, 3H), 3.66-3.64 (m, 2H), 3.05-3.00 (m, 2H), 2.34 (s, 3H)

M⁻(ES): 426.2; M⁺(ES): 428.2; HPLC (max plot) 89.06%; Rt: 3.35 min.

### Example 28

### methyl 4-({4-[1,3-benzoxazol-2(3H)-ylidene(cyano)methyl]-2-pyrimidinyl}amino)butanoate

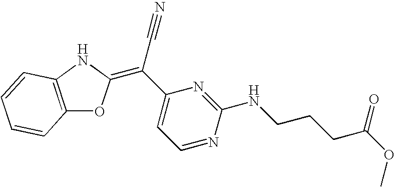

Following the general strategies and protocols outlined in the procedure B, the title compound was obtained from 1,3-benzoxazol-2(3H)-ylidene(2-chloro-4-pyrimidinyl)-ethanenitrile and methyl-4-aminobutyrate.HCl in the presence of triethylamine for 6 min at 155° C. in MeOH (79%).

¹H NMR (DMSO-d₆) δ 12.93-11.1 (m, 1H), 8.67-6.35 (m, 7H), 3.58 (s, 3H), 3.40-3.29 (m, 2H), 2.44-2.36 (m, 2H), 1.90-1.81 (m, 2H)

M⁻(ES): 350.2; M⁺(ES): 352.3; HPLC (max plot) 81.61%; Rt: 2.55 min

### Example 29

### (2-amino-4-pyrimidinyl)(1,3-benzoxazol-2(3H)-ylidene)ethanenitrile

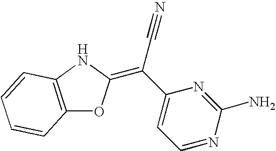

Following the general strategies and protocols outlined in the procedure B, the title compound was obtained from (2-amino-4-pyrimidinyl)(1,3-benzoxazol-2(3H)-ylidene)ethanenitrile (245.00 mg; 0.91 mmol) and ammonium hydroxyde (0.70 ml; 18.15 mmol) for 30 min at 160° C. in iPrOH (87%).

¹H NMR (CDCl₃) δ 7.73 (d, J=6.03 Hz, 1H), 7.45-7.41 (m, 2H), 7.22-7.16 (m, 2H), 6.59 (d, J=6.03 Hz, 1H), 5.24 (s, 2H)

M⁻(ES): 250.2; M⁺(ES): 252.2; HPLC (max plot) 83.56%; Rt: 2.08 min.

### Example 30

### methyl 4-[({1,3-benzoxazol-2(3H)-ylidene(cyano)methyl]-2-pyrimidinyl}amino)methyl]benzoate

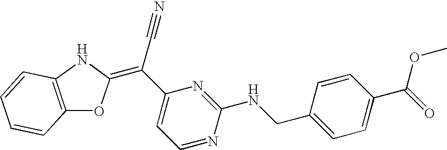

Following the general strategies and protocols outlined in the procedure B, the title compound was obtained from 1,3-benzoxazol-2(3H)-ylidene(2-chloro-4-pyrimidinyl)ethanenitrile and methyl-4-(aminomethyl)benzoate.HCl in the presence of triethylamine for 5 min at 155° C. in MeOH (83%).

¹H NMR (DMSO-d₆) δ 13.5-6.0 (m, 12H), 4.90-4.50 (m, 2H), 3.83 (s, 3H)

M⁻(ES): 398.1; M⁺(ES): 399.8; HPLC (max plot) 96.37%; Rt: 3.21 min.

### Example 31

### tert-butyl 4-[({4-[1,3-benzoxazol-2(3H)-ylidene(cyano)methyl]-2-pyrimidinyl}amino)methyl]-1-piperidinecarboxylate

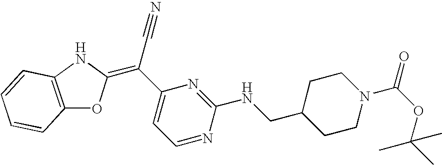

Following the general strategies and protocols outlined in the procedure B, the title compound was obtained from 1,3-benzoxazol-2(3H)-ylidene(2-chloro-4-pyrimidinyl)ethanenitrile and 4-(aminomethyl)-1-N-Boc-piperidine in the presence of triethylamine for 5 min at 155° C. in EtOH (86%).

¹H NMR (DMSO-d₆) δ 12.87-10.98 (m, 1H), 8.73-6.35 (m, 7H), 3.96-3.92 (m, 2H), 3.31-3.20 (m, 1H), 2.78-2.59 (m, 2H), 1.87-1.65 (m, 3H), 1.38 (s, 9H), 1.16-1.03 (m, 3H)

M⁻(ES): 447.1; M⁺(ES): 449.1; HPLC (max plot) 99.63%; Rt: 3.39 min.

### Example 32

### 1,3-benzoxazol-2(3H)-ylidene{2-[(2-hydroxyethyl)amino]-4-pyrimidinyl}ethanenitrile

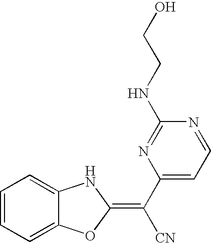

Following the general strategies and protocols outlined in the procedure B, the title compound was obtained from 1,3-benzoxazol-2(3H)-ylidene(2-chloro-4-pyrimidinyl)ethanenitrile and ethanolamine in the presence of triethylamine for 5 min at 155° C. in EtOH (82%).

¹H NMR (DMSO-d₆) δ 12.97-10.99 (m, 1H), 8.79-6.35 (m, 7H), 4.91 (s, 1H), 3.58 (s, 2H), 3.44-3.38 (s, 2H)

HPLC (max plot) 97.5%; Rt: 2.08 min.

### Example 33

### methyl 4-({4-[1,3-benzoxazol-2(3H)-ylidene(cyano)methyl]-5-methyl-2-pyrimidinyl}amino)butanoate

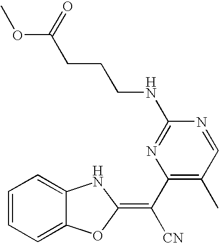

Following the general strategies and protocols outlined in the procedure B, the title compound was obtained from 1,3-benzoxazol-2(3H)-ylidene(2-chloro-5-methyl-4-pyrimidinyl)ethanenitrile and methyl-4-aminobutyrate.HCl in the presence of triethylamine for 12 min at 155° C. in MeOH (84%).

¹H NMR (DMSO-d₆) δ 7.8-7 (m, 6H), 3.57 (s, 3H), 3.5-3.35 (m, 2H), 2.47-2.37 (m, 2H), 2.33 (s, 3H), 1.95-1.80 (m, 2H)

M⁻(ES): 364.1; M⁺(ES): 366.1; HPLC (max plot) 84%; Rt: 2.70 min

### Example 34

### 1,3-benzoxazol-2(3H)-ylidene(2-{[3-(4-methyl-2-oxo-1-piperazinyl)propyl]amino}-4-pyrimidinyl)ethanenitrile

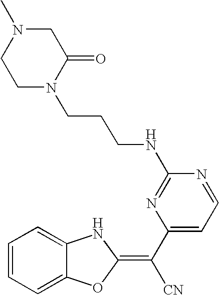

Following the general strategies and protocols outlined in the procedure B, the title compound was obtained from 1,3-benzoxazol-2(3H)-ylidene(2-chloro-4-pyrimidinyl)ethanenitrile and 1-(3-amino-propyl)-4-methyl-piperazi-2-one.HCl in the presence of triethylamine for 4×10 min at 155° C. in EtOH (13.4%).

¹H NMR (DMSO-d₆) δ 14.00-6.45 (m, 8H), 3.87 (s, 2H), 3.57-3.36 (m, 8H), 2.86 (s, 3H), 1.82 (s, 2H)

M⁻(ES): 404.3; M⁺(ES): 406.3; HPLC (max plot) 99.9%; Rt: 1.86 min.

General Procedure C

10 mg of Building Blocks were dissolved in 0.3 mL of DMA. Et₃N (4eq.) and the amines (4 eq.) dissolved in DMA (0.3 mL) were then added to the reaction mixtures and the plate was sealed and heated in a microwave (Mars 5) as follows: 2 plates at a time were heated 4 min at 300 Watts and then left to cool down for 10 min. This was repeated 4 times. The reaction mixtures were then transferred into a 2 mL plate and the solvent was removed in the Genevac. Work up: 1 mL of water/CH₃COOH (2%) was then added and the plate was shaken for 3 h00. The aqueous layer was removed using the Zymark, leaving the solid behind. This solid was further washed with water (2×). 1 mL of MeOH/TFA (20%) was added to the plates, which were shaken at rt for 48 h and the supernatant was collected using the Lissy. Analytical plates were made and the solvents were removed in the Genevac.

### Example 35

### 1,3-benzoxazol-2(3H)-ylidene{2-[(2-pyridin-2-ylethyl)amino]pyrimidin-4-yl}acetonitrile

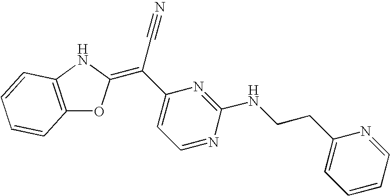

Following the general strategies and protocols outlined in the procedure C, the title compound was obtained from 1,3-benzoxazol-2(3H)-ylidene(2-chloro-pyrimidin-4-yl)acetonitrile and 2-(2-aminoethyl)pyridine in the presence of triethylamine in DMA.

M⁺(ES): 357.2; LC (215 nm): 64%, Rt: 1.38 min

### Example 36

### 1,3-benzoxazol-2(3H)-ylidene[2-(isopropylamino)pyrimidin-4-yl]acetonitrile

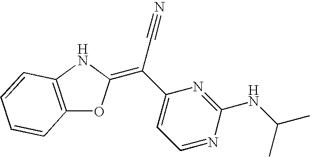

Following the general strategies and protocols outlined in the procedure C, the title compound was obtained from 1,3-benzoxazol-2(3H)-ylidene(2-chloro-pyrimidin-4-yl)acetonitrile and isopropylamine in the presence of triethylamine in DMA.

M⁺(ES): 294.2; LC (215 nm): 61%, Rt: 1.54 min

### Example 37

### 1,3-benzoxazol-2(3H)-ylidene{2-[(2,3-dimethylcyclohexyl)amino]pyrimidin-4-yl}acetonitrile

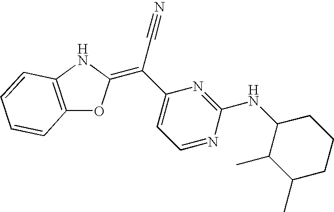

Following the general strategies and protocols outlined in the procedure C, the title compound was obtained from 1,3-benzoxazol-2(3H)-ylidene(2-chloro-pyrimidin-4-yl)acetonitrile and (2,3-dimethylcyclohexyl)amine in the presence of triethylamine in DMA.

M⁺(ES): 362.2; LC (215 nm): 52%, Rt: 1.82 min

### Example 38

### 1,3-benzoxazol-2(3H)-ylidene{2-[(1-methylbutyl)amino]pyrimidin-4-yl}acetonitrile

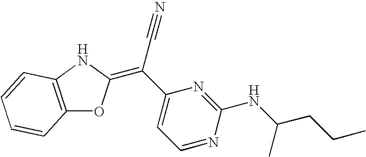

Following the general strategies and protocols outlined in the procedure C, the title compound was obtained from 1,3-benzoxazol-2(3H)-ylidene(2-chloro-pyrimidin-4-yl)acetonitrile and (1-methylbutyl)amine in the presence of triethylamine in DMA.

M⁺(ES): 322.2; LC (215 nm): 66%, Rt: 1.76 min

### Example 39

### 1,3-benzoxazol-2(3H)-ylidene{2-[(pyridin-2-ylmethyl)amino]pyrimidin-4-yl}acetonitrile

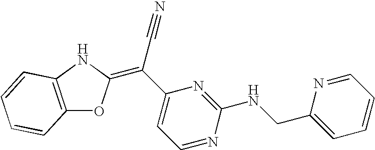

Following the general strategies and protocols outlined in the procedure C, the title compound was obtained from 1,3-benzoxazol-2(3H)-ylidene(2-chloro-pyrimidin-4-yl)acetonitrile and (pyridin-2-ylmethyl)amine in the presence of triethylamine in DMA.

M⁺(ES): 343.2; LC (215 nm): 90%, Rt: 1.49 min

### Example 40

### 1,3-benzoxazol-2(3H)-ylidene{2-[(3-butoxypropyl)amino]pyrimidin-4-yl}acetonitrile

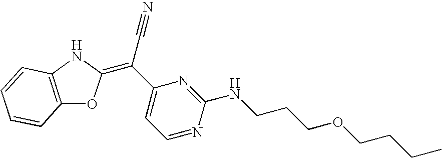

Following the general strategies and protocols outlined in the procedure C, the title compound was obtained from 1,3-benzoxazol-2(3H)-ylidene(2-chloro-pyrimidin-4-yl)acetonitrile and (3-butoxypropyl)amine in the presence of triethylamine in DMA.

M⁺(ES): 366.3; LC (215 nm): 75.5%, Rt: 1.73 min

### Example 41

### 1,3-benzoxazol-2(3H)-ylidene{2-[(pyridin-3-ylmethyl)amino]pyrimidin-4-yl}acetonitrile

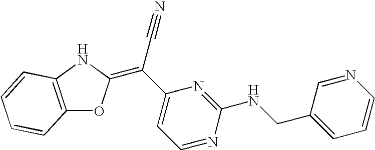

Following the general strategies and protocols outlined in the procedure C, the title compound was obtained from 1,3-benzoxazol-2(3H)-ylidene(2-chloro-pyrimidin-4-yl)acetonitrile and (pyridin-3-ylmethyl)amino in the presence of triethylamine in DMA.

M⁺(ES): 343.2; LC (215 nm): 82%, Rt: 1.41 min

### Example 42

### 1,3-benzoxazol-2(3H)-ylidene{2-[(3-isopropoxypropyl)amino]pyrimidin-4-yl}acetonitrile

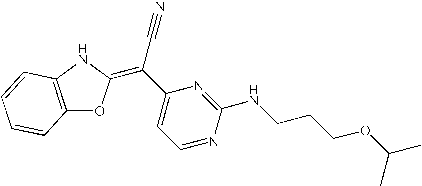

Following the general strategies and protocols outlined in the procedure C, the title compound was obtained from 1,3-benzoxazol-2(3H)-ylidene(2-chloro-pyrimidin-4-yl)acetonitrile and (3-isopropoxypropyl)amine in the presence of triethylamine in DMA.

M⁺(ES): 352.2; LC (215 nm): 76%, Rt: 1.71 min

### Example 43

### 1,3-benzoxazol-2(3H)-ylidene{2-[(1-ethylpropyl)amino]pyrimidin-4-yl}acetonitrile

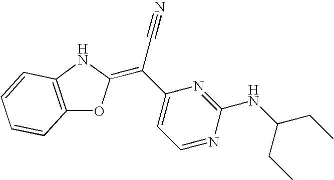

Following the general strategies and protocols outlined in the procedure C, the title compound was obtained from 1,3-benzoxazol-2(3H)-ylidene(2-chloro-pyrimidin-4-yl)acetonitrile and (1-ethylpropy)amine in the presence of triethylamine in DMA.

M⁺(ES): 322.2; LC (215 nm): 39%, Rt: 1.67 min

### Example 44

### 1,3-benzoxazol-2(3H)-ylidene{2-[ethyl(isopropyl)amino]pyrimidin-4-yl}acetonitrile

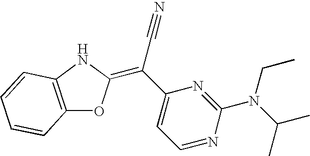

Following the general strategies and protocols outlined in the procedure C, the title compound was obtained from 1,3-benzoxazol-2(3H)-ylidene(2-chloro-pyrimidin-4-yl)acetonitrile and N-ethylpropan-2-amine in the presence of triethylamine in DMA.

M⁺(ES): 322.2; LC (215 nm): 30%, Rt: 1.89 min

### Example 45

### 1,3-benzoxazol-2(3H)-ylidene[2-(cyclopentylamino)pyrimidin-4-yl]acetonitrile

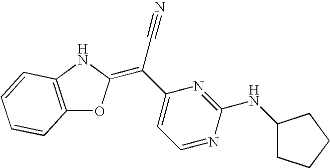

Following the general strategies and protocols outlined in the procedure C, the title compound was obtained from 1,3-benzoxazol-2(3H)-ylidene(2-chloro-pyrimidin-4-yl)acetonitrile and cyclopentylamine in the presence of triethylamine in DMA.

M⁺(ES): 320.2; LC (215 nm): 41.5%, Rt: 1.64 min

### Example 46

### 1,3-benzoxazol-2(3H)-ylidene[2-(cyclohexylamino)pyrimidin-4-yl]acetonitrile

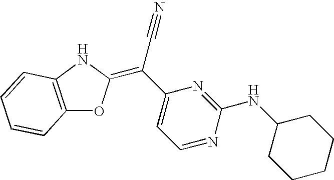

Following the general strategies and protocols outlined in the procedure C, the title compound was obtained from 1,3-benzoxazol-2(3H)-ylidene(2-chloro-pyrimidin-4-yl)acetonitrile and cyclohexylamine in the presence of triethylamine in DMA.

M⁺(ES): 334.2; LC (215 nm): 34%, Rt: 1.77 min

### Example 47

### 1,3-benzoxazol-2(3H)-ylidene(6-methyl-2-{[3-(1H-1,2,4-triazol-1-yl)propyl]amino}pyrimidin-4-acetonitrile

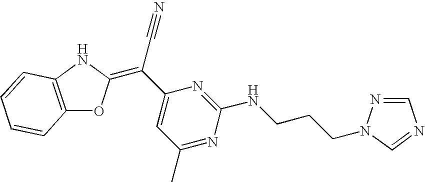

Following the general strategies and protocols outlined in the procedure C, the title compound was obtained from 1,3-benzoxazol-2(3H)-ylidene(2-chloro-6-methylpyrimidin-4-yl)acetonitrile and [3-(1H-1,2,4-triazol-1yl)propyl]amine in the presence of triethylamine in DMA.

M⁺(ES): 375.3; LC (215 nm): 56.5%, Rt: 1.56 min

### Example 48

### 1,3-benzoxazol-2(3H)-ylidene[2-(cyclopentylamino)-6-methylpyrimidin-4-yl]acetonitrile

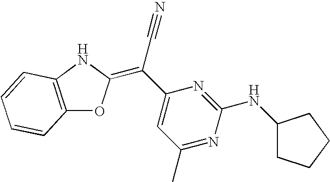

Following the general strategies and protocols outlined in the procedure C, the title compound was obtained from 1,3-benzoxazol-2(3H)-ylidene(2-chloro-6-methylpyrimidin-4-yl)acetonitrile and cyclopentylamine in the presence of triethylamine in DMA.

M⁺(ES): 334.2; LC (215 nm): 44%, Rt: 1.72 min

### Example 49

### 1,3-benzoxazol-2(3H)-ylidene[6-(4-ethylpiperazin-1-yl)pyrimidin-4-yl]acetonitrile

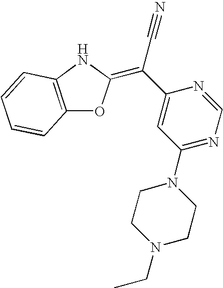

Following the general strategies and protocols outlined in the procedure C, the title compound was obtained from 1,3-benzoxazol-2(3H)-ylidene(6-chloro-pyrimidin-4-yl)acetonitrile and 1-ethylpiperazine in the presence of triethylamine in DMA.

M⁺(ES): 349.3; LC (215 nm): 34.5%, Rt: 1.62 min

### Example 50

### 1,3-benzoxazol-2(3H)-ylidene[2-(cyclohexylamino)-6-methylpyrimidin-4-yl]acetonitrile

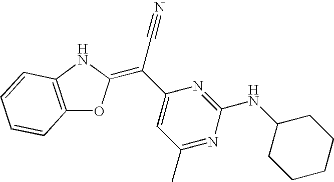

Following the general strategies and protocols outlined in the procedure C, the title compound was obtained from 1,3-benzoxazol-2(3H)-ylidene(2-chloro-6-methylpyrimidin-4-yl)acetonitrile and cyclohexylamine in the presence of triethylamine in DMA.

M⁺(ES): 348.2; LC (215 nm): 51%, Rt: 1.79 min

### Example 51

### 1,3-benzoxazol-2(3H)-ylidene{2-[benzyl(isopropyl)amino]pyrimidin-4-yl}acetonitrile

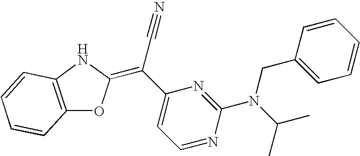

Following the general strategies and protocols outlined in the procedure C, the title compound was obtained from 1,3-benzoxazol-2(3H)-ylidene(2-chloropyrimidin-4-yl)acetonitrile and N-benzylpropan-2-amine in the presence of triethylamine in DMA.

M⁺(ES): 384.2; LC (215 nm): 35%, Rt: 2.05 min

### Example 52

### 1,3-benzoxazol-2(3H)-ylidene[6-(cyclopentylamino)pyrimidin-4-yl]acetonitrile

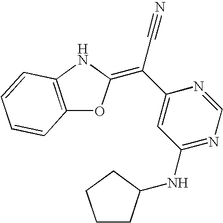

Following the general strategies and protocols outlined in the procedure C, the title compound was obtained from 1,3-benzoxazol-2(3H)-ylidene(6-chloro-pyrimidin-4-yl)acetonitrile and cyclopentylamine in the presence of triethylamine in DMA.

M⁺(ES): 320.2; LC (215 nm): 35%, Rt: 1.94 min.

Procedure D

### Example 53

### 1,3-benzoxazol-2(3H)-ylidene(2-{[4-(4-methyl-1-piperazinyl)-4-oxobutyl]amino}-4-pyrimidinyl)ethanenitrile

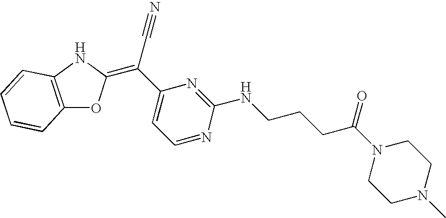

A suspension of methyl 4-({4-[1,3-benzoxazol-2(3H)-ylidene(cyano)methyl]-2-pyrimidinyl}amino)butanoate (200.00 mg, 0.57 mmol) in neat 1-methylpiperazine (2 ml) was heated up to 150° C. for 20 minutes in the microwave device on normal absorption. The mixture was evaporated to dryness. The residue was taken up in Ether/EtOH 9:1. The resulting precipitate was filtered off, washed with ether then dried at 40° C. under vacuum. The residue was taken up in DCM to which TFA was added. Ether in excess was added and the precipitate obtained was filtered off and washed with ether (3×) then dried under vacuum at 40° C. This solid was purified by HPLC preparative to afford a solid after lyophilisation. It was then solubilized in MeOH and evaporated under reduced pressure to give the title compound as a yellow solid (35%).

¹H NMR (DMSO-d₆) δ 12.96-11.39 (m, 1H), 9.87-8.75 (m, 1H), 7.92-6.26 (m, 5H), 4.55-4.35 (m, 2H), 4.15-4.00 (m, 2H), 3.50-3.25 (m, 4H), 3.08-2.85 (m, 2H), 2.79 (s, 3H), 2.55-2.40 (m, 2H), 1.90-1.75 (m, 2H)

M⁻(ES): 418.1; M⁺(ES): 419.9; HPLC (max plot) 100%; Rt: 1.97 min.

### Example 54

### 1,3-benzoxazol-2(3H)-ylidene(2-{[4-(4-morpholinyl)-4-oxobutyl]amino}-4-pyrimidinyl)ethanenitrile

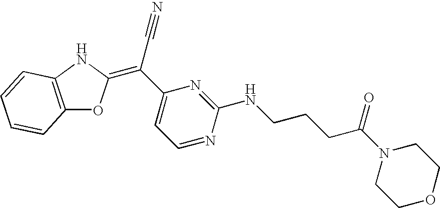

Following the general strategies and protocols outlined in the procedure D, the title compound was obtained from methyl methyl 4-({4-[1,3-benzoxazol-2(3H)-ylidene(cyano)methyl]-2-pyrimidinyl}amino)butanoate in neat morpholine for 20 minutes at 150° C. (67%).

¹H NMR (Methanol-d₄) δ 7.74 (d, J=6.1 Hz, 1H), 7.39-7.30 (m, 2H), 7.25-7.00 (m, 3H), 3.70-3.52 (m, 8H), 3.50-3.40 (m, 2H), 2.60-2.48 (m, 2H), 2.20-1.87 (m, 2H).

M⁻(ES): 405.4; M⁺(ES): 407.2; HPLC (max plot) 95%; Rt: 2.51 min.

### Example 55

### 1,3-benzoxazol-2(3H)-ylidene(2-{[4-oxo-4-(1-piperidinyl)butyl]amino}-4-pyrimidinyl)ethanenitrile

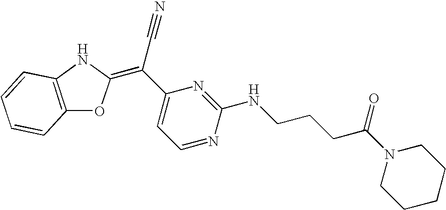

Following the general strategies and protocols outlined in the procedure D, the title compound was obtained from methyl methyl 4-({4-[1,3-benzoxazol-2(3H)-ylidene(cyano)methyl]-2-pyrimidinyl}amino)butanoate in neat piperidine for 20 minutes at 150° C. (53%).

¹H NMR (Methanol-d₄) δ 7.75 (d, J=6.0 Hz, 1H), 7.40-7.31 (m, 2H), 7.22-7.00 (m, 3H), 3.63-3.40 (m, 6H), 2.60-2.48 (m, 2H), 2.05-1.88 (m, 2H), 1.75-1.45 (m, 6H)

M⁻(ES): 403.2; M⁺(ES): 405.3; HPLC (max plot) 98%; Rt: 2.99 min.

### Example 56

### 1,3-benzoxazol-2(3H)-ylidene[2-({4-[4-(2-methoxyethyl)-1-piperazinyl]-4-oxobutyl}amino)-4-pyrimidinyl]ethanenitrile

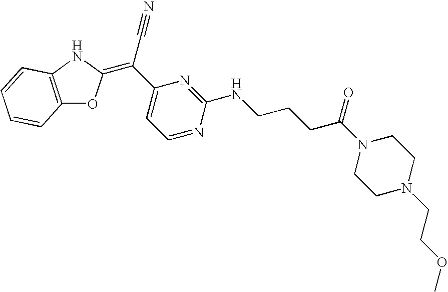

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate in neat 1-(2-methoxyethyl)-piperazine for 20 minutes at 150° C. (48%).

¹H NMR (MeOD) δ: 8.00-7.78 (br s, 1H), 7.75-7.55 (m, 2H), 7.55-7.35 (m, 2H), 6.9-6.65 (m, 1H), 4-3.62 (m, 2H), 3.62-3.05 (m, 28H), 2.78-2.48 (t, 2H), 2.21-1.83 (q, 2H).

M⁻(ES): 462.3; M⁺(ES): 464.4; HPLC (max plot) 95.8%; Rt: 199 min.

### Example 57

### 1,3-benzoxazol-2(3H)-ylidene(2-{[4-(1,4-dioxa-8-azaspiro[4.5]dec-8-yl)-4-oxobutyl]amino}-4-pyrimidinyl)ethanenitrile

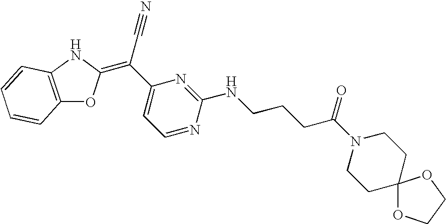

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and 1,4-dioxa-8-azaspiro[4.5]decane (13.7Eq) in THF (1 ml) for 20 minutes at 150° C. (55%).

¹H NMR (MeOD) δ 7.75 (d, J=6.0 Hz, 1H), 7.40-7.32 (m, 2H), 7.22-7.12 (m, 2H), 7.10-7.00 (m, 1H), 3.97 (s, 4H), 3.75-3.57 (m, 4H), 3.50-3.40 (m, 2H), 2.62-2.52 (m, 2H), 2.22-1.85 (m, 2H), 1.75-1.60 (m, 4H).

M⁻(ES): 461.4; M⁺(ES): 463.3; HPLC (max plot) 98.9%; Rt: 2.74 min.

### Example 58

### 1,3-benzoxazol-2(3H)-ylidene(2-{[4-oxo-4-(1-piperazinyl)butyl]amino}-4-pyrimidinyl)ethanenitrile

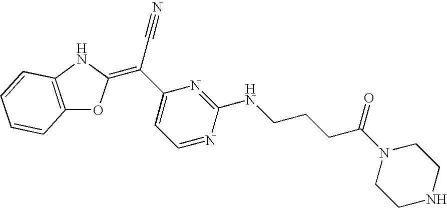

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and piperazine (10Eq) in THF (3 ml) for 20 minutes at 150° C. (20%).

¹H NMR (MeOD) δ 8.1-7.8 (s, 1H), 7.8-7.6 (m, 2H), 7.58-7.37 (m, 2H), 6.95-6.66 (s, 1H), 4-3.8 (m, 4H), 3.7-3.5 (m, 2H), 3.4-3.23 (m, 4H), 2.74-2.65 (t, 2H), 2.17-2.04 (m, 2H).

M⁻(ES): 404.2; M⁺(ES): 406.2; HPLC (max plot) 98.4%; Rt: 1.89 min.

### Example 59

### 4-({4-[1,3-benzoxazol-2(3H)-ylidene(cyano)methyl]-2-pyrimidinyl}amino)-N,N-bis(2-methoxyethyl)butanamide

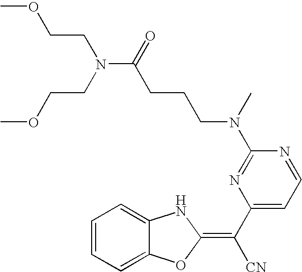

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate in neat bis(2-methoxyethyl)amine for 4×20 minutes at 150° C. (12%).

¹H NMR (Methanol-d₄) δ: 7.88 (s, 1H), 7.75-7.55 (m, 2H), 7.55-7.30 (m, 2H), 6.85-6.60 (s, 1H), 3.70-3.40 (m, 10H), 2.68-2.64 (t, J=6.8 Hz, 2H), 2.05-1.96 (q, J=6.8 Hz, 2H)

M⁻(ES): 451.2; M⁺(ES): 453.1; HPLC (max plot) 99.4%; Rt: 2.71 min.

### Example 60

### 1,3-benzoxazol-2(3H)-ylidene(2-{[4-(4-hydroxy-1-piperidinyl)-4-oxobutyl]amino}-4-pyrimidinyl)ethanenitrile

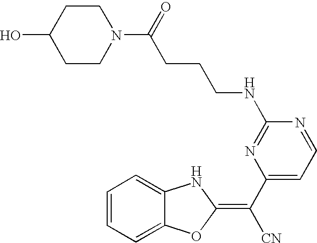

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and 4-hydroxypiperidine (5Eq) in THF (3 ml) for 3×15 minutes at 140° C. (36%).

¹H NMR (Methanol-d₄) δ 8-7.3 (m, 5H), 6.75 (s, 1H), 4.20-4 (m, 1H), 3.90-3.76 (m, 2H), 3.60-3.40 (m, 2H), 3.26-3.12 (m, 2H), 2.62-2.58 (t, J=6.8 Hz, 2H), 2.10-1.95 (q, 2H), 1.95-1.75 (m, 2H), 1.60-1.35 (m, 2H).

M⁻(ES): 419.2; M⁺(ES): 421.3; HPLC (max plot) 99%; Rt: 2.32 min.

### Example 61

### 1,3-benzoxazol-2(3H)-ylidene(2-{[4-(4-isopropyl-1-piperazinyl)-4-oxobutyl]amino}-4-pyrimidinyl)ethanenitrile

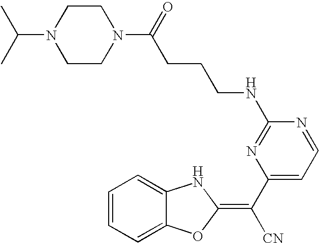

Following the general strategies and protocols outlined in the procedure D, the title compound was obtained from methyl 4-({4-[1,3-benzoxazol-2(3H)-ylidene(cyano)-methyl]-2-pyrimidinyl}amino)butanoate in neat 1-isopropyl-piperazine for 20 minutes at 150° C. (27%).

¹H NMR (Methanol-d₄) δ: 8-7.2 (m, 5H), 6.6-6.8 (m, 1H), 4.4-4.1 (m, 1H), 3.8-3.4 (m, 6H), 3.4-2.9 (m, 4H), 2.8-2.55 (m, 2H), 2.15-2.00 (m, 2H), 1.41-1.38 (d, J=6.7 Hz, 6H)

M⁻(ES): 446.3; M⁺(ES): 448.4; HPLC (max plot) 97.4%; Rt: 2.06 min.

### Example 62

### 1,3-benzoxazol-2(3H)-ylidene(2-{[4-(4-ethyl-1-piperazinyl)-4-oxobutyl]amino}-4-pyrimidinyl)ethanenitrile

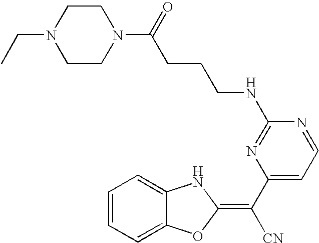

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and 1-ethylpiperazine (5Eq) in THF (3 ml) for 7×20 minutes at 150° C. (23%).

¹H NMR (Methanol-d₄) δ: 8-7.2 (m, 5H), 6.75 (m, 1H), 4.4-4.1 (m, 1H), 3.9-2.85 (m, 11H), 2.8-2.5 (t, J=6.1 Hz, 2H), 2.2-1.9 (q, J=7 Hz, 2H), 1.5-1.25 (t, J=7.4 Hz, 3H)

M⁻(ES): 423.4; M⁺(ES): 434.3; HPLC (max plot) 99.7%; Rt 1.94 min.

### Example 63

### 1,3-benzoxazol-2(3H)-ylidene(2-{[4-(4-cyclohexyl-1-piperazinyl)-4-oxobutyl]amino}-4-pyrimidinyl)ethanenitrile

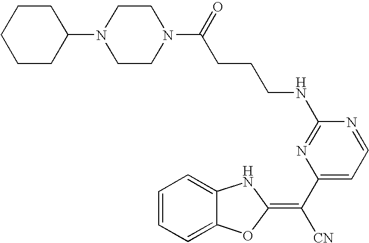

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and 1-cyclohexylpiperazine (5Eq) in THF (3 ml) for 5×20 minutes at 150° C. (23%).

¹H NMR (Methanol-d₄) δ: 7.96-7.82 (m, 1H), 7.72-7.66 (m, 2H), 7.49-7.40 (m, 2H), 6.84-6.69 (m, 1H), 4.80-4.65 (m, 1H), 4.38-4.16 (m, 1H), 3.70-2.94 (m, 8H), 2.75-2.60 (m, 2H), 2.16-1.94 (m, 6H), 1.76-1.72 (m, 1H), 1.55-1.20 (m, 6H).

M⁻(ES): 486.5; M⁺(ES): 488.5; HPLC (max plot) 97%; Rt 2.32 min.

### Example 64

### 1,3-benzoxazol-2(3H)-ylidene(5-methyl-2-{[4-(4-methyl-1-piperazinyl)-4-oxobutyl]amino}-4-pyrimidinyl)ethanenitrile

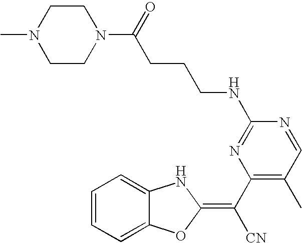

Following the general strategies and protocols outlined in the procedure D, the title compound was obtained from methyl 4-({4-[1,3-benzoxazol-2(3H)-ylidene(cyano)methyl]-5-methyl-2-pyrimidinyl}amino)butanoate in neat 1-methylpiperazine for 20 minutes at 150° C. (5%).

¹H NMR (Methanol-d₄) δ: 7.67-7.64 (m, 3H), 7.47-7.38 (m, 2H), 3.60-3.10 (m, 8H), 3.54-3.49 (m, 2H), 2.96 (s, 3H), 2.67-2.62 (m, 2H), 2.48 (s, 3H), 2.10-2.03 (m, 2H).

M⁻(ES): 432.2; M⁺(ES): 434.3; HPLC (max plot) 100%; Rt: 2.03 min.

### Example 65

### 1,3-benzoxazol-2(3H)-ylidene[2-({4-[4-(2-hydroxyethyl)-1-piperazinyl]-4-oxobutyl}amino)-4-pyrimidinyl]ethanenitrile

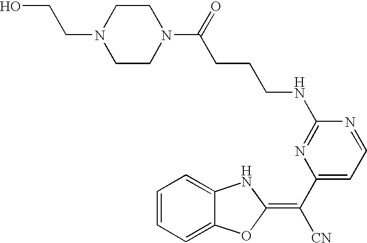

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and N-(2-hydroxyethyl)piperazine (5Eq) in THF (3 ml) for 2×40 minutes at 150° C. (21%).

¹H NMR (Methanol-d₄) δ: 7.80-7.68 (d, J=6 Hz, 1H), 7.37-7.27 (m, 2H), 7.20-6.95 (m, 3H), 3.70-3.50 (m, 6H), 3.50-3.35 (t, J=6.4 Hz, 2H), 2.60-2.35 (m, 8H), 2.00-1.85 (q, J=6.4,14 Hz, 2H)

M⁻(ES): 448.3; M⁺(ES): 450.4; HPLC (max plot) 99.9%; Rt 1.89 min.

### Example 66

### 1,3-benzoxazol-2(3H)-ylidene(2-{[4-oxo-4-(4-phenyl-1-piperazinyl)butyl]amino}-4-pyrimidinyl)ethanenitrile

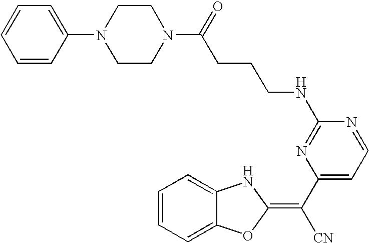

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and 1-phenylpiperazine (5Eq) in THF (3 ml) for 5×20 minutes at 140° C. (45%).

¹H NMR (Methanol-d₄) δ: 7.8-7.65 (d, J=6 Hz, 1H), 7.45-7.3 (dd, J=3.4; 7.5 Hz, 2H), 7.25-6.75 (m, 8H), 3.8-3.65 (m, 4H), 3.55-3.4 (t, J=6.7 Hz, 2H), 3.15-3.05 (m, 4H), 2.65-2.53 (t, J=7.5 Hz, 2H), 2.07-1.95 (q, J=6.4; 7.5 Hz, 2H)

M⁻(ES): 480.3; M⁺(ES): 482.4; HPLC (max plot) 99.6%; Rt 2.91 min.

### Example 67

### 4-({4-[1,3-benzoxazol-2(3H)-ylidene(cyano)methyl]-2-pyrimidinyl}amino)-N,N-bis(2-hydroxyethyl)butanamide

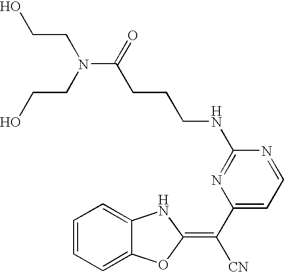

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and diethanolamine (5Eq) in THF (3 ml) for 4×40 minutes at 150° C. (21%).

¹H NMR (Methanol-d₄) δ: 7.75-7.65 (m, 1H), 7.35-6.90 (m, 5H), 3.75-3.60 (m, 4H), 3.60-3.42 (m, 4H), 253.30 (m, 2H), 2.60-2.49 (m, 2H), 1.98-1.82 (m, 2H)

M⁻(ES): 423.3; M⁺(ES): 425.3; HPLC (max plot) 96.3%; Rt 2.11 min.

### Example 68

### 1,3-benzoxazol-2(3H)-ylidene[2-({4-oxo-4-[4-(2-pyridinyl)-1-piperazinyl]butyl}amino)-4-pyrimidinyl]ethanenitrile

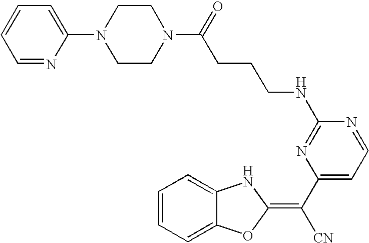

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and 1-(2-pyridyl)piperazine (5Eq) in THF (3 ml) for 40+80 minutes at 150° C. (52%).

¹H NMR (Methanol-d₄) δ: 8.15-7.95 (m, 2H), 7.95-7.80 (s, 1H), 7.75-7.57 (m, 2H), 7.52-7.30 (m, 3H), 7.10-6.95 (t, 1H), 6.80-6.65 (s, 1H), 3.95-3.72 (m, 8H), 3.65-3.45 (m, 2H), 2.75-2.62 (t, J=6.8 Hz, 2H), 2.15-2.00 (q, J=6.8 Hz, 2H)

M⁻(ES): 481.4; M⁺(ES): 483.3; HPLC (max plot) 100%; Rt 2.11 min.

### Example 69

### 1,3-benzoxazol-2(3H)-ylidene{2-[(4-oxo-4-{4-[2-oxo-2-(1-pyrrolidinyl)ethyl]-1-piperazinyl}butyl)amino]-4-pyrimidinyl}ethanenitrile

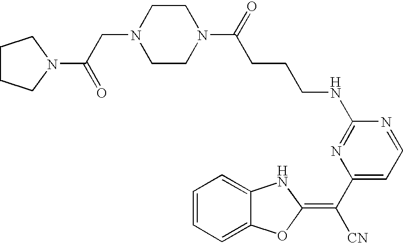

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and 1-(pyrrolidinocarbonylmethyl)piperazine (5Eq) in THF (3 ml) for 40 minutes at 150° C. (52%).

¹H NMR (Methanol-d₄) δ: 8.00-7.75 (m, 1H), 7.75-7.55 (t, 2H), 7.55-7.35 (q, 2H), 6.85-6.65 (m, 1H), 4.22 (s, 2H), 4.10-3.80 (m, 4H), 3.65-3.37 (m, 10H), 2.75-2.57 (t, J=6.8 Hz, 2H), 2.15-1.85 (m, 6H)

M⁻(ES): 515.4; M⁺(ES): 517.4; HPLC (max plot) 99.5%; Rt 2.11 min.

### Example 70

### (2-{[4-(4-acetyl-1-piperazinyl)-4-oxobutyl]amino}-4-pyrimidinyl)(1,3-benzoxazol-2(3H)-ylidene)ethanenitrile

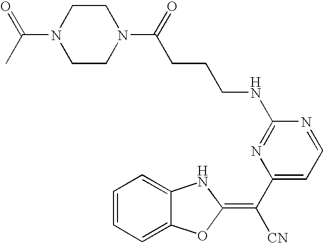

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and 1-acetylpiperazine (5Eq) in THF (3 ml) for 40+6×80 minutes at 150° C. (68.6%).

¹H NMR (Methanol-d₄) δ: 7.80-7.70 (d, J=6 Hz, 1H), 7.40-7.30 (d, J=7.9 Hz, 2H), 7.25-6.95 (m, 3H), 3.75-3.50 (m, 8H), 3.50-3.40 (t, J=6.4 Hz, 2H), 2.65-2.50 (t, J=7.5 Hz, 2H), 2.13-2.02 (d, J=14.4 Hz, 3H), 2.02-1.85 (m, 2H)

M⁻(ES): 446.3; M⁺(ES): 448.3; HPLC (max plot) 99.9%; Rt 2.33 min.

### Example 71

### ethyl {4-[4-({4-[1,3-benzoxazol-2(3H)-ylidene(cyano)methyl]-2-pyrimidinyl}amino)butanoyl]-1-piperazinyl}acetate

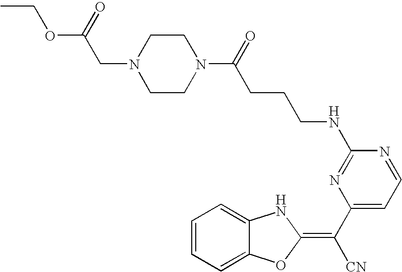

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and 1-(ethoxycarbonylmethyl)piperazine (5Eq) in THF (3 ml) for 40+4×80 minutes at 150° C. (10.3%).

¹H NMR (Methanol-d₄) δ: 8.00-7.30 (m, 5H), 6.85-6.60 (m, 1H), 4.40-4.25 (quadruplet, J=7.2 Hz, 2H), 4.09 (s, 2H), 3.95-3.80 (m, 4H), 3.65-3.45 (m, 2H), 3.45-3.20 (m, 4H), 2.70-2.57 (t, J=7.2 Hz, 2H), 2.15-1.95 (m, 2H), 1.40-1.25 (t, J=7.1 Hz, 3H)

M⁻(ES): 490.4; M⁺(ES): 492.4; HPLC (max plot) 99.4%; Rt 2.15 min.

### Example 72

### 1,3-benzoxazol-2(3H)-ylidene(2-{[4-(4-benzyl-1-piperazinyl)-4-oxobutyl]amino}-4-pyrimidinyl)ethanenitrile

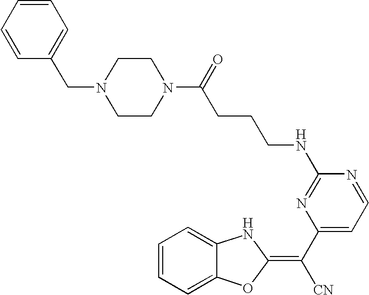

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and 1-benzylpiperazine (5Eq) in THF (3 ml) for 40+2×80 minutes at 150° C. (54%).

¹H NMR (Methanol-d₄) δ: 8.00-7.80 (m, 1H), 7.75-7.40 (m, 9H), 6.85-6.70 (m, 1H), 4.40 (s, 2H), 4.10-3.75 (m, 2H), 3.75-3.20 (m, 8H), 2.70-2.55 (t, J=6.8 Hz, 2H), 2.15-1.95 (m, 2H)

M⁻(ES): 494.3; M⁺(ES): 496.3; HPLC (max plot) 99.9%; Rt 2.38 min.

### Example 73

### 1,3-benzoxazol-2(3H)-ylidene[2-({4-oxo-4-[4-(2-pyrimidinyl)-1-piperazinyl]butyl}amino)-4-pyrimidinyl]ethanenitrile

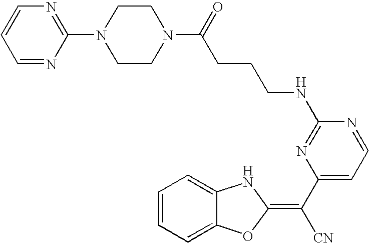

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and 1-(2-pyrimidyl)piperazine (5Eq) in THF (3 ml) for 40+5×80 minutes at 150° C. (22%).

¹H NMR (Methanol-d₄) δ: 8.33-8.25 (d, J=4.5 Hz, 2H), 7.77-7.68 (d, J=6 Hz, 1H), 7.38-7.28 (m, 2H), 7.20-7.09 (m, 2H), 7.09-6.97 (m, 1H), 6.60-6.52 (t, J=4.9 Hz, 1H), 3.87-3.72 (m, 4H), 3.72-3.57 (m, 4H), 3.52-3.42 (t, J=6.4 Hz, 2H), 2.65-2.53 (t, J=7.5 Hz, 2H), 2.06-1.90 (m, 2H)

M⁻(ES): 482.3; M⁺(ES): 484.3; HPLC (max plot) 99.7%; Rt 2.57 min.

### Example 74

### 1,3-benzoxazol-2(3H)-ylidene[2-({4-[4-(2-methoxyethyl)-1-piperazinyl]-4-oxobutyl}amino)-5-methyl-4-pyrimidinyl]ethanenitrile

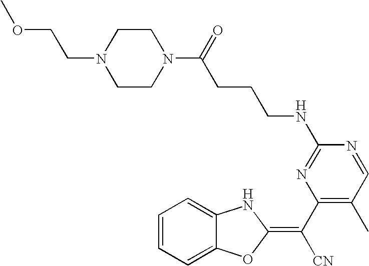

Following the general strategies and protocols outlined in the procedure D, the title compound was obtained from methyl 4-({4-[1,3-benzoxazol-2(3H)-ylidene(cyano)methyl]-2-pyrimidinyl}amino)butanoate and 1-(2-methoxyethyl)-piperazine (5Eq) in THF (3 ml) for 3×20 minutes at 150° C. (80%).

¹H NMR (Methanol-d₄) δ: 7.62 (s, 1H), 7.35-7.22 (m, 2H), 7.17-6.95 (m, 2H), 3.60-3.53 (m, 2H), 3.53-3.44 (m, 4H), 3.44-3.35 (t, J=6.4 Hz, 2H), 3.35-3.28 (s, 3H), 2.60-2.35 (m, 8H), 2.20 (s, 3H), 2.00-1.85 (m, 2H)

M⁻(ES): 476.4; M⁺(ES): 478.4; HPLC (max plot) 96.2%; Rt 2.16 min.

Procedure E

### Example 75

### 4-[({4-[1,3-benzoxazol-2(3H)-ylidene(cyano)methyl]-5-methyl-2-pyrimidinyl}amino)methyl]benzoic acid

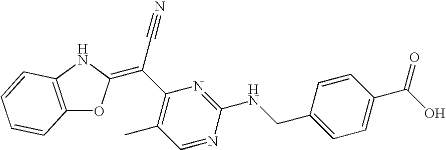

A solution of methyl 4-[({4-[1,3-benzoxazol-2(3H)-ylidene(cyano)methyl]-5-methyl-2-pyrimidinyl}amino)methyl]benzoate trifluoroacetate (120.00 mg; 0.23 mmol) in EtOH (5.00 ml) and NaOH (0.23 ml; 5.00 M; 1.14 mmol) was heated up to 50° C. overnight. The solvent was evaporated and an excess of water was added. The solution was neutralized with a solution of HCl 1N. The precipitate obtained was filtrated off, washed with water then was dried under vacuum to afford the title compound (92%).

¹H NMR (DMSO-d₆) δ 14.02 (br s, 1Hexchangeable), 9.03 (br s, 1Hexchangeable), 7.91 (d, J=8.3 Hz, 2H), 7.76 (s, 1H), 7.67 (d, J=7.9 Hz, 1H), 7.57 (d, J=7.9 Hz, 1H), 7.49 (d, J=8.3 Hz, 2H), 7.38-7.25 (m, 2H), 4.70-4.68 (m, 2H), 2.34 (s, 3H).

M⁻(ES): 398.2; M⁺(ES): 400.1; HPLC (max plot) 93.30%; Rt: 2.78 min.

### Example 76

### 4-[2-({4-[1,3-benzoxazol-2(3H)-ylidene(cyano)methyl]-2-pyrimidinyl}amino)ethyl]benzoic acid

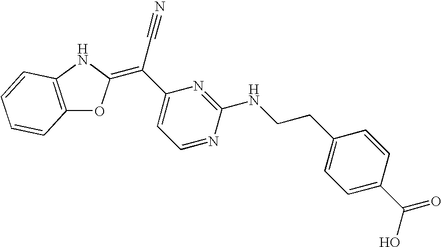

Following the general strategies and protocols outlined in the procedure E, the title compound was obtained from methyl 4-[2-({4-[1,3-benzoxazol-2(3H)-ylidene(cyano)methyl]-2-pyrimidinyl}amino)ethyl]benzoate in the presence of NaOH for 3 h at room temperature in EtOH (90%).

HPLC (max plot) 89.77%; Rt: 2.72 min.

### Example 77

### 4-[({4-[1,3-benzoxazol-2(3H)-ylidene(cyano)methyl]-2-pyrimidinyl}amino)methyl]benzoic acid

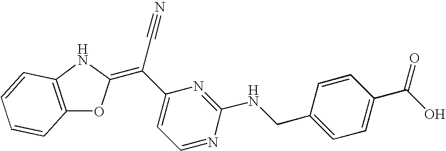

Following the general strategies and protocols outlined in the procedure E, the title compound was obtained methyl 4-[({4-[1,3-benzoxazol-2(3H)-ylidene(cyano)methyl]-2-pyrimidinyl}amino)methyl]benzoate in the presence of NaOH overnight at room temperature in EtOH (97%).

¹H NMR (DMSO-d₆) δ 14.00-6.00 (m, 11H), 5.00-4.50 (m, 2H)

M⁻(ES): M⁺(ES): HPLC (max plot) 92.05%; Rt: 2.63 min.

### Example 78

### 4-({4-[1,3-benzoxazol-2(3H)-ylidene(cyano)methyl]-2-pyrimidinyl}-amino)butanoic acid

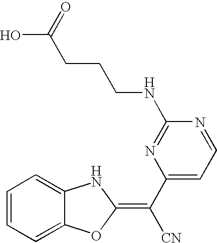

Following the general strategies and protocols outlined in the procedure E, the title compound was obtained from methyl 4-({4-[(Z)-1,3-benzoxazol-2(3H)-ylidene(cyano)methyl]-2-pyrimidinyl}amino)butanoate in the presence of NaOH 1 hour at 50° C. in EtOH (34%).

¹H NMR (DMSO-d₆) δ 7.68-6.90 (m, 6H), 6.31 (s, 1H), 3.27-3.16 (m, 3H), 2.26-2.21 (t, 2H), 1.78-1.68 (t, 2H)

HPLC (max plot) 100%; Rt: 2.30 min.

Procedure F

### Example 79

### 1,3-benzoxazol-2(3H)-ylidene[5-methyl-2-({4-[(4-methyl-1-piperazinyl)carbonyl]benzyl}amino)-4-pyrimidinyl]ethanenitrile

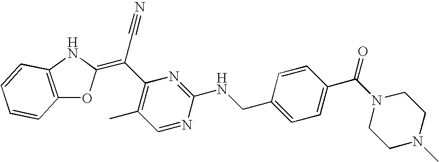

To a solution of 1-methylpiperazine (0.02 ml; 0.21 mmol), 4-[({4-[1,3-benzoxazol-2(3H)-ylidene(cyano)methyl]-5-methyl-2-pyrimidinyl}amino)methyl]benzoic acid (83.30 mg; 0.21 mmol), EDC-HCl (43.98 mg, 0.23 mmol) and HOBT (31.00 mg; 0.23 mmol) in DCM (7.00 ml) was added DIEA (0.05 ml; 0.31 mmol) and the reaction mixture was stirred at rt overnight. The reaction mixture was then diluted with DCM and washed with a saturated solution of NaHCO3, NH4Cl and brine. The organic layer was dried over MgSO4 and evaporated then dried under vacuum. The residue was taken up in DCM to which TFA was added. Ether in excess was added and the precipitate obtained was filtered off and washed with ether (3×) then dried under vacuum at 40° C. The solid was purified by preparative HPLC to afford, after lyophilisation, the title compound as a yellow powder (21%).

¹H NMR (DMSO-d₆) δ 9.76 (m, 1H), 8.94 (m, 1H), 7.77-7.25 (m, 9H), 4.67 (m, 2H), 3.55-3.00 (m, 8H), 2.80 (s, 3H), 2.34 (s, 3H)

M⁻(ES): 480.0; M⁺(ES): 482.1; HPLC (max plot) 100%; Rt: 2.13 min.

### Example 80

### 1,3-benzoxazol-2(3H)-ylidene{2-[(2-{4-[(4-methyl-1-piperazinyl)carbonyl]-phenyl}ethyl)amino]-4-pyrimidinyl}ethanenitrile

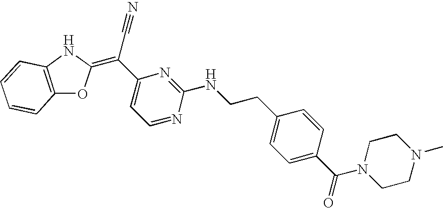

Following the general strategies and protocols outlined in the procedure F, the title compound was obtained from 4-[2-({4-[1,3-benzoxazol-2(3H)-ylidene(cyano)methyl]-2-pyrimidinyl}amino)ethyl]benzoic acid and 1-methylpiperazine in the presence of EDC—HCl, HOBT and DIEA for 5 days at room temperature in DCM (24%).

¹H NMR (DMSO-d₆) δ 11.29-6.38 (m, 11H), 3.66-2.93 (m, 12H), 2.79 (s, 3H)

M⁻(ES): 480.1; M⁺(ES): 482.1; HPLC (max plot) 99.07%; Rt: 2.08 min.

### Example 81

### 4-[2-({4-[1,3-benzoxazol-2(3H)-ylidene(cyano)methyl]-2-pyrimidinyl}-amino)ethyl]-N-[2-(dimethylamino)ethyl]benzamide

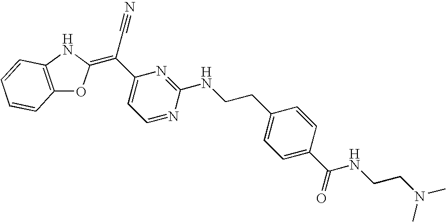

Following the general strategies and protocols outlined in the procedure F, the title compound was obtained from 4-[2-({4-[1,3-benzoxazol-2(3H)-ylidene(cyano)methyl]-2-pyrimidinyl}amino)ethyl]benzoic acid and 2-dimethylaminoethylamine in the presence of EDC—HCl, HOBT and DIEA for 5 days at room temperature and one day at 50° C. in DCM (38%).

¹H NMR (DMSO-d₆) δ 11.4-6.40 (m, 12H), 3.66-3.55 (m, 4H), 3.25-3.24 (m, 2H), 2.96-2.98 (m, 2H), 2.84-2.82 (m, 5H)

M⁻(ES): M⁺(ES): HPLC (max plot) 97.99%; Rt: 2.17 min.

### Example 82

### 1,3-benzoxazol-2(3H)-ylidene[2-({4-[(4-methyl-1-piperazinyl)carbonyl]-benzyl}amino)-4-pyrimidinyl]ethanenitrile

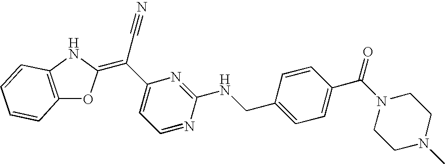

Following the general strategies and protocols outlined in the procedure F, the title compound was obtained 4-[({4-[1,3-benzoxazol-2(3H)-ylidene(cyano)methyl]-2-pyrimidinyl}amino)methyl]benzoic acid and 1-methylpiperazine in the presence of EDC—HCl, HOBT and DIEA for 6 days at room temperature in DCM (53%).

¹H NMR (DMSO-d₆) δ 11.80-6.10 (m, 13H), 5.50-3.65 (m, 4H), 3.60-3.00 (m, 6H), 2.80 (s, 3H)

M⁻(ES): M⁺(ES): HPLC (max plot) 100%; Rt: 2.01 min.

### Example 83

### 1,3-benzoxazol-2(3H)-ylidene(2-{[4-(4-fluoro-1-piperidinyl)-4-oxobutyl]amino}-4-pyrimidinyl)ethanenitrile

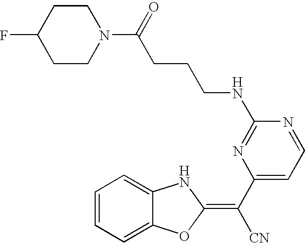

Following the general strategies and protocols outlined in the procedure F, the title compound was obtained 4-({4-[1,3-benzoxazol-2(3H)-ylidene(cyano)methyl]-2-pyrimidinyl}amino)butanoic acid and 4-fluoropiperidine in the presence of EDC—HCl, HOBT and DIEA for 1.5 days at room temperature in DCM (5%).

¹H NMR (Methanol-d₄) δ 7.75-7.68 (d, J=6 Hz, 1H), 7.33-7.31 (d, 2H), 7.2-6.9 (m, 3H), 4.95-4.65 (m, 1H), 3.80-3.47 (m, 4H), 3.47-3.35 (t, 2H), 2.60-2.48 (m, 2H), 2.00-1.86 (m, 4H), 1.86-1.65 (m, 2H)

M⁻(ES): 421.1; M⁺(ES): 423.2; HPLC (max plot) 100%; Rt: 2.78 min.

Procedure G

### Example 84

### 1,3-benzoxazol-2(3H)-ylidene{5-methyl-2-[(4-piperidinylmethyl)amino]-4-pyrimidinyl}ethanenitrile

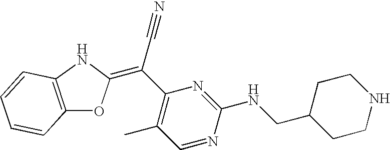

To a solution of tert-butyl 4-[({4-[1,3-benzoxazol-2(3H)-ylidene(cyano)methyl]-5-methyl-2-pyrimidinyl}amino)methyl]-1-piperidinecarboxylate (136.70 mg; 0.30 mmol) in DCM (4.50 ml) was added TFA (0.5 ml) and the solution was stirred for 1 h at room temperature. Ether in excess was added and the precipitate obtained was filtered off and washed with ether (3×) then dried under vacuum at 40° C., affording the title compound as a yellow solid (94%).

HPLC (max plot) 96.74%; Rt: 1.99 min.

### Example 85

### 1,3-benzoxazol-2(3H)-ylidene{2-[(4-piperidinylmethyl)amino]-4-pyrimidinyl}ethanenitrile

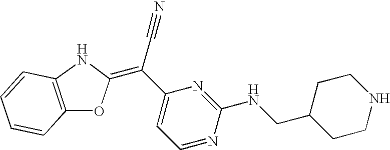

Following the general strategies and protocols outlined in the procedure G, the title compound was obtained from tert-butyl 4-[({4-[1,3-benzoxazol-2(3H)-ylidene(cyano)methyl]-2-pyrimidinyl}amino)methyl]-1-piperidinecarboxylate in the presence TFA for 1 night at room temperature in DCM (108%).

¹H NMR (DMSO-d₆) δ 13.00-11.43 (m, 1H), 8.86-6.40 (m, 7H), 3.56-3.27 (m, 4H), 2.90-2.80 (m, 2H), 1.90-1.83 (m, 3H), 1.40-1.28 (m, 2H)

M⁻(ES): 347.2; M⁺(ES): 349.1; HPLC (max plot) 99.75%; Rt: 1.87 min.

Procedure H

### Example 86

### (2-{[(1-acetyl-4-piperidinyl)methyl]amino}-4-pyrimidinyl)(1,3-benzoxazol-2(3H)-ylidene)ethanenitrile

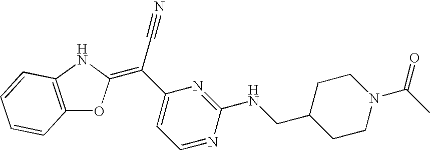

To a solution of 1,3-benzoxazol-2(3H)-ylidene{2-[(4-piperidinylmethyl)amino]-4-pyrimidinyl}ethanenitrile bis(trifluoroacetate) (200.00 mg, 0.35 mmol) in DMA (3 ml) at 0° C. were added Et₃N (0.19 ml; 1.39 mmol) and acetyl chloride (0.02 ml; 0.35 mmol) and the resulting solution was stirred at 0° C. for 10 minutes then at rt for 4 h. Another Eq of acetyl chloride was added and the mixture was stirred for 1 hour. The solvent was evaporated with the Genevac. The residue was taken up in DCM to which TFA was added. Ether in excess was added and the precipitate obtained was filtered off and washed with ether (3×) then dried under vacuum at 40° C. The crude solid was purificated by preparative HPLC to afford, after lyophilisation, the title compound as a yellow powder (55%).

¹H NMR (Methanol-d₄) δ: 7.71-7.45 (m, 5H), 6.76 (br s, 1H), 4.65-4.60 (m, 1H), 4.06-4.01 (m, 3H), 3.41-3.40 (m, 2H), 3.24-3.16 (m, 1H), 2.75-2.67 (m, 1H), 2.15 (s, 3H), 2.10-1.94 (m, 3H), 1.44-1.25 (m, 1H)

M⁻(ES): 389.2; M⁺(ES): 391.2; HPLC (max plot) 100%; Rt: 2.47 min.

### Example 87

### 1,3-benzoxazol-2(3H)-ylidene{2-[({1-[(dimethylamino)acetyl]-4-piperidinyl}methyl)amino]-4-pyrimidinyl}ethanenitrile

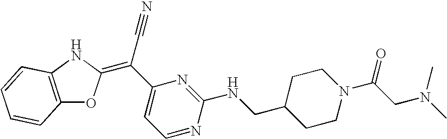

Following the general strategies and protocols outlined in the procedure H, the title compound was obtained from 1,3-benzoxazol-2(3H)-ylidene{2-[(4-piperidinylmethyl)-amino]-4-pyrimidinyl}ethanenitrile bis(trifluoroacetate) and dimethylaminoacetyl chloride hydrochloride in the presence triethylamine for 2 days at room temperature in DMA (22%)

¹H NMR (DMSO-d₆) δ 12.9-6.38 (m, 8H), 4.38-4.33 (m, 1H), 4.28-4.22 (m, 2H), 3.62-3.58 (m, 1H), 3.30 (s, 2H), 3.06-2.98 (m, 1H), 2.79 (s, 6H), 2.69-2.65 (m, 1H), 1.90-1.75 (m, 3H), 1.21-1.05 (m, 2H)

M⁻(ES): M⁺(ES): HPLC (max plot) 86.43%; Rt: 1.96 min.

### Example 88

### (2-{[(1-acetyl-4-piperidinyl)methyl]amino}-5-methyl-4-pyrimidinyl)(1,3-benzoxazol-2(3H)-ylidene)ethanenitrile

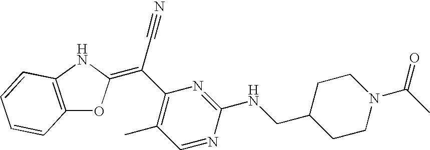

Following the general strategies and protocols outlined in the procedure H, the title compound was obtained from 1,3-benzoxazol-2(3H)-ylidene{5-methyl-2-[(4-piperidinylmethyl)amino]-4-pyrimidinyl}ethanenitrile bis(trifluoroacetate) and acetyl chloride in the presence triethylamine for 6 hours at room temperature in DMA (50%).

¹H NMR (DMSO-d₆) δ 8.49 (br s, 1H), 7.76 (s, 1H), 7.69-7.67 (m, 1H), 7.57-7.55 (m, 1H), 7.38-7.26 (m, 2H), 4.39-4.34 (m, 1H), 3.83-3.79 (m, 1H), 3.29-3.28 (m, 2H), 3.01-2.94 (m, 1H), 2.34 (s, 3H), 1.96 (s, 3H), 1.90-1.72 (m, 3H), 1.22-0.99 (m, 3H)

M⁻(ES): 403.2; M⁺(ES): 405.1; HPLC (max plot) 100%; Rt: 2.61 min.

Procedure I

### Example 89

### N-{4-[1,3-benzoxazol-2(3H)-ylidene(cyano)methyl]-2-pyrimidinyl}-4-(dimethylamino)butanamide

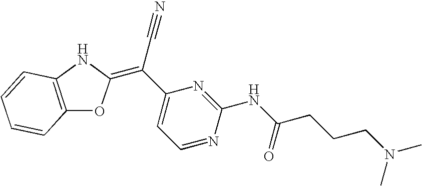

To a suspension of (2-amino-4-pyrimidinyl)(1,3-benzoxazol-2(3H)-ylidene)ethanenitrile (198.00 mg; 0.66 mmol), 4-dimethylaminobutyric acid.HCl (166.46 mg; 0.99 mmol) and 2-chloro-1-methylpyridinium iodide (507.37 mg; 1.99 mmol) in DCM/THF (3/1, 16 ml) was added N-ethyldiisopropylamine (0.38 ml; 2.19 mmol). The reaction mixture was stirred at room temperature overnight, then diluted with DCM, washed with NaHCO3 sat. and brine. The organic layer was dried over MgSO4 and evaporated. The solid was taken up in DCM to which TFA was added. Ether in excess was added and the precipitate obtained was filtered off and washed with ether (3×) then dried under vacuum at 40° C. The solid was purified by preparative HPLC to afford, after lyophilisation, the title as a yellow powder (48%).

¹H NMR (Methanol-d₄) δ: 8.12 (d, J=5.7 Hz, 1H), 7.54-7.49 (m, 2H), 7.37-7.26 (m, 2H), 6.86 (d, J=5.7 Hz, 1H), 3.35-3.27 (m, 2H), 2.98 (s, 6H), 2.73-2.69 (m, 2H), 2.24-2.17 (m, 2H)

M⁻(ES): 363.1; M⁺(ES): 365.1; HPLC (max plot) 99.14%; Rt: 1.86 min.

Procedure J

### Example 90

### N-{4-[1,3-benzoxazol-2(3H)-ylidene(cyano)methyl]-2-pyrimidinyl}-1-methyl-4-piperidinecarboxamide

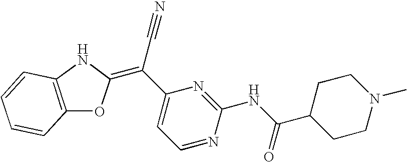

To a suspension of (2-amino-4-pyrimidinyl)(1,3-benzoxazol-2(3H)-ylidene)ethanenitrile (100.00 mg; 0.40 mmol), 1-methyl-piperidine-4-carboxylic acid HCl (107.25 mg; 0.60 mmol) and 2-chloro-1-methylpyridinium iodide (203.37 mg; 0.80 mmol) in THF (4.00 ml) was added DIEA (0.34 ml; 1.99 mmol) and the resulting suspension was heated up to 150° C. under microwave conditions during 900 s (normal absorption, 9 bar). After ON standing at 4° C., the precipitate formed was filtered off and washed thoroughly with THF then water. After drying at 40° C. for 2 days, the solid was taken up in DCM to which TFA was added. Ether in excess was added and the precipitate obtained was filtered off and washed with ether (3×) then dried under vacuum at 40° C. The solid was purified by preparative HPLC to afford after lyophilisation the title compound as a yellow fluffy solid (19%).

¹H NMR (Methanol-d₄) δ: 8.15-7.95 (m, 1H), 7.47-7.10 (m, 4H), 6.83-6.65 (m, 1H), 3.65-3.30 (m, 4H), 3.16-3.08 (m, 3H), 3.07-2.90 (m, 1H), 2.42-1.90 (m, 4H)

M⁻(ES): 375.1; M⁺(ES): 377.1; HPLC (max plot) 98.1%; Rt: 2.00 min.

Procedure K

### Example 91

### 2-{4-[({4-[1,3-benzoxazol-2(3H)-ylidene(cyano)methyl]-2-pyrimidinyl}amino)methyl]-1-piperidinyl}-N,N-dimethylacetamide

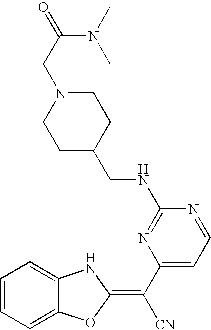

To a suspension of 1,3-benzoxazol-2(3H)-ylidene{2-[(4-piperidinylmethyl)amino]-4-pyrimidinyl}ethanenitrile bis(trifluoroacetate) (330.00 mg; 0.57 mmol; 1.00 eq.) in 8 ml DMA were added dropwise at 0° C., triethylamine (0.48 ml; 3.43 mmol; 6.00 eq.) and a solution of 2-chloro-N,N-dimethylacetamide (83.51 mg; 0.69 mmol; 1.20 eq.) in 3 ml of DMA. The mixture was stirred overnight at rt. The solvent was evaporated and water then NaHCO3 10% were added. The product was extracted with DCM. The combined organic layers were washed with brine (4×), dried over magnesium sulfate, filtered and concentrated to dryness. The resulting solid was taken up in DCM to which an excess of TFA was added followed by addition of an excess of Ether. The precipitate obtained was filtered off and washed with ether (3×) then dried under vacuum at 40° C. overnight, affording 112 mg (30%) of the title compound as a yellow powder.

1H NMR (Methanol-d₄) δ: 8-7.2 (m, 5H), 6.8-6.6 (m, 1H), 4.22 (s, 2H), 3.85-3.65 (m, 2H), 3.55-3.40 (m, 2H), 3.2-3.05 (m, 2H), 3.06-3.02 (s, 6H), 2.25-2.00 (m, 3H), 1.75-1.60 (m, 2H)

M⁻(ES): 432.4; M⁺(ES): 434.4; HPLC (max plot) 98.4%; Rt: 1.99 min.

### Example 92

### 2-{4-[({4-[1,3-benzoxazol-2(3H)-ylidene(cyano)methyl]-5-methyl-2-pyrimidinyl}amino)methyl]-1-piperidinyl}-N,N-dimethylacetamide

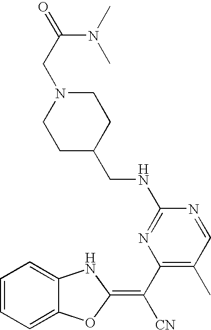

Following the general strategies and protocols outlined in the procedure K, the title compound was obtained from 1,3-benzoxazol-2(3H)-ylidene{5-methyl-2-[(4-piperidinylmethyl)amino]-4-pyrimidinyl}ethanenitrile bis(trifluoroacetate) and 2-chloro-N,N-dimethylacetamide (1Eq) in presence of triethylamine in DMA (11 ml) for 3 days at rt (52%).

¹H NMR (Methanol-d₄) δ: 9.35 (br s, 1H), 8.54 (br s, 1H), 7.76 (s, 1H), 7.68 (d, J=7.91 Hz, 1H), 7.58-7.56 (m, 1H), 7.38-7.26 (m, 2H), 4.27-4.15 (m, 2H), 3.80-3.41 (m, 2H9, 3.35-3.18 (m, 2H), 2.99-2.87 (m, 2H), 2.90 (s, 3H), 2.88 (s, 3H), 2.34 (s, 3H), 2.01-1.80 (m, 3H), 1.63-1.48 (m, 2H).

M⁻(ES): 446.2; M⁺(ES): 448.3; HPLC (max plot) 99.5%; Rt 2.10 min.

### Example 93

### Preparation of a Pharmaceutical Formulation

The following formulation examples illustrate representative pharmaceutical compositions according to the present invention being not restricted thereto.

Formulation 1—Tablets

A benzoxazole acetonitrile of formula I is admixed as a dry powder with a dry gelatin binder in an approximate 1:2 weight ration. A minor amount of magnesium stearate is added as a lubricant. The mixture is formed into 240-270 mg tablets (80-90 mg of active benzoxazole acetonitrile compound per tablet) in a tablet press.

Formulation 2—Capsules

A benzoxazole acetonitrile of formula I is admixed as a dry powder with a starch diluent in an approximate 1:1 weight ratio. The mixture is filled into 250 mg capsules (125 mg of active benzoxazole acetonitrile compound per capsule).

Formulation 3—Liquid

A benzoxazole acetonitrile of formula I (1250 mg), sucrose (1.75 g) and xanthan gum (4 mg) are blended, passed through a No. 10 mesh U.S. sieve, and then mixed with a previously prepared solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

Formulation 4—Tablets

A benzoxazole acetonitrile of formula I is admixed as a dry powder with a dry gelatin binder in an approximate 1:2 weight ratio. A minor amount of magnesium stearate is added as a lubricant. The mixture is formed into 450-900 mg tablets (150-300 mg of active benzoxazole acetonitrile compound) in a tablet press.

Formulation 5—Injection

A benzoxazole acetonitrile of formula (I) is dissolved in a buffered sterile saline injectable aqueous medium to a concentration of approximately 5 mg/ml.

Biological Assays

The compounds of the present invention may be subjected to the following assays:

a) GSK3 in vitro Assay:

GSK3β Assay (see Bioorg. Med. Chem. Lett by Naerum et al. 12 p. 1525-1528 (2002))

In a final reaction volume of 25 μl, GSK3β (h) (5-10 mU) is incubated with 8 mM MOPS pH 7.0, 0.2 mM EDTA, 20 μM YRRAAVPPSPSLSRHSSPHQS(p)EDEEE (being the GSK3 substrate; a phospho GS2 peptide), 10 mM Mg Acetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and the degree of phosphorylation of the substrate is determined by scintillation counting.

The tested compounds according to formula I typically display an inhibition (IC₅₀) with regard to GSK3 of less than 20 μM, preferably less than 10 and even more preferred less than 1 μM.

The binding affinities of the compounds of formula (I) were assessed using the above described in vitro biological assay. Representative values for some example compounds are given in Tables 1 and 2 below.

The values in Table 1 refer to the binding affinity (IC₅₀; μM) of typical example compounds according to formula I to GSK3.

TABLE 1
In vitro potency of benzoxazole derivatives on human GSK3 beta

| | | IC₅₀ (μM) |
|---|---|---|
| Structure | Compound | GSK3beta |
| 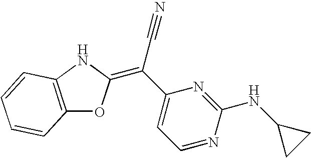 | 1,3-benzoxazol-2(3H)-ylidene[2- (cyclopropylamino)pyrimidin-4-yl]acetonitrile | <10 |
| 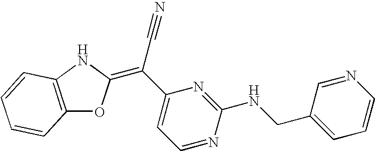 | 1,3-benzoxazol-2(3H)-ylidene{2-[(pyridin-3- ylmethyl)amino]pyrimidin-4-yl}acetonitrile | <10 |
| 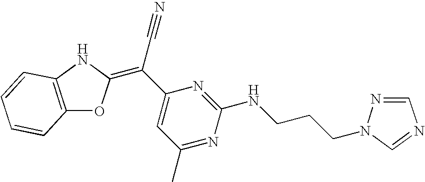 | 1,3-benzoxazol-2(3H)-ylidene(6-methyl-2-{[3-(1H- 1,2,4-triazol-1-yl) propyl]amino} pyrimidin-4- yl)acetonitrile | <10 |
| 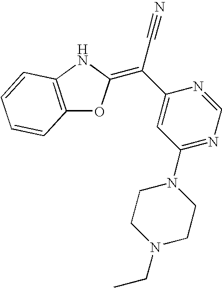 | 1,3-benzoxazol-2(3H)-ylidene[6-(4-ethylpiperazin-1- yl)pyrimidin-4-yl]acetonitrile | <10 |
| 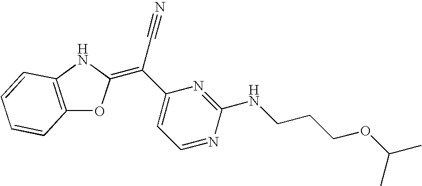 | 1,3-benzoxazol-2(3H)-ylidene{2-[(3- isopropoxypropyl)amino]pyrimidin-4-yl}acetonitrile | <10 |
| 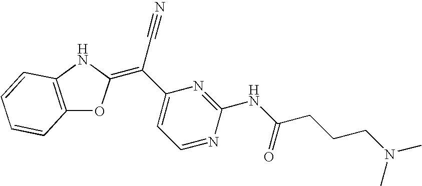 | N-{4-[1,3-benzoxazol-2(3H)-ylidene(cyano)methyl]- 2-pyrimidinyl}-4-(dimethylamino)butanamide | <10 |
| 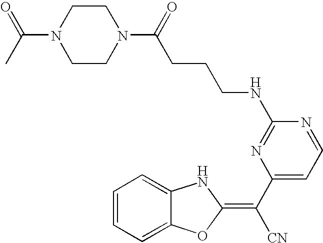 | (2-{[4-(4-acetyl-1-piperazinyl)-4-oxobutyl]amino}- 4-pyrimidinyl)(1,3-benzoxazol-2(3H)-ylidene)- ethanenitrile | <10 |

b) In vivo Assay: Experimental Model of Type II Diabetes (Oral Postprandial Glycemia in db/db Mice)

The following assay aims at determining the anti-diabetic effect of the test compounds of formula (I) in a model of postprandial glycemia in db/db mice, in vivo.

The assay was performed as follows:

A total of 24 db/db mice (about 8-9 weeks; obtained from IFFACREDO, l'Arbreste, France) were fasted during 20 hours.

2 groups, each consisting of 6 animals were formed:
Group 1: The animals were administered (per os) a dose of 10 mg/kg of vehicle.
Group 2: The animals were administered (per os) a dose of 50 mg/kg of the test compound according to formula (I).

After oral administration of the compounds of formula (I) solubilized or suspended in CarboxyMethylCellulose (0.5%), Tween 20 (0.25%) and water as vehicle, the animals had access to commercial food (D04, UAR, Villemoisson/Orge, France) ad libitum. The diabetic state of the mice was verified by determining the blood glucose level before drug administration. Blood glucose and serum insulin levels were then determined 4 hrs after drug administration.

The determination of the blood glucose level was performed using a glucometer (Precision Q.I.D., Medisense, Abbot, ref. 212.62.31).

The determination of the insulin level was performed using an ELISA kit (Crystal CHEM, Ref. INSK R020).

Changes in blood glucose and serum insulin of drug treated mice were expressed as a percentage of control (group 1: vehicle treated mice).

Treatment (per os) of the animals with substituted benzoxazole acetonitrile compounds of formula (I), at a dosage of 50 mg/kg, decreased the blood glucose level induced by food intake by about 20-40%.

For instance, upon administering the compound of Example 5, i.e 1,3-benzoxazol-2(3H)-ylidene(2-{[3-(2-oxopyrrolidin-1-yl)propyl]amino}pyrimidin-4-yl)acetonitrile (p.o. 50 mg/kg), the blood glucose level was found to be reduced at about 25% and the insulin level was found to be reduced at about 11%, compared to the animals of Group 1.

### REFERENCE LIST

1. Woodgett et al: Trends Biochem. Sci., 16 p. 177-81 (1991);

2. Reaven et al (American Journal of Medicine, 60, 80 (1976);

3. Stout, Metabolism, 34, 7 (1985)

4. Diamanti-Kandarakis et al.; European Journal of Endocrinology 138, 269-274 (1998),

5. Andrea Dunaif; Endocrine Reviews 18(6), 774-800 (1997));

6. WO 01/47920

1. A benzoxazole acetonitrile according to formula (I) 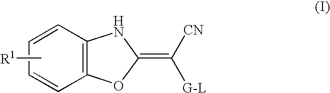
as well as its tautomers, its geometrical isomers, its optically active forms as enantiomers, diastereomers and its racemate forms, as well as pharmaceutically acceptable salts thereof, wherein
G is pyrimidinyl;
L is an amino group, a 3-8 membered heterocycloalkyl comprising at least one heteroatom selected from the group consisting of N, O, and S, or L is an acylamino moiety;
R¹ is selected from the group consisting of hydrogen, sulfonyl, amino, C₁-C₆-alkyl, C₂-C₆-alkenyl, C₂-C₆-alkynyl, C₁-C₆-alkoxy, aryl, halogen, carboxy, aminocarbonyl, cyano and hydroxy.

2. The benzoxazole acetonitrile according to claim 1, wherein R¹ is H or C₁-C₃ alkyl.

3. The benzoxazole acetonitrile according to claim 1, having the formulae 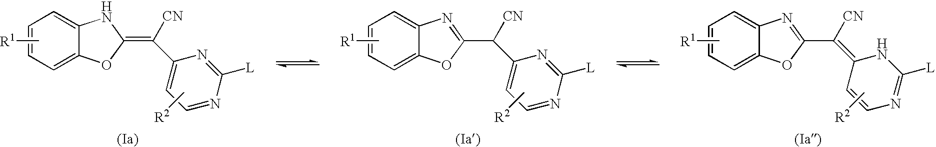
wherein R¹ is selected from the group consisting of hydrogen, sulfonyl, amino, C₁-C₆-alkyl, C₂-C₆-alkenyl, C₂-C₆-alkynyl, C₁-C₆-alkoxy, aryl, halogen, carboxy, aminocarbonyl, cyano and hydroxy and
L is an amino group of the formula —NR³R⁴, wherein R³ and R⁴ are each independently from each other H, C₁-C₆-alkyl, C₂-C₆-alkenyl, C₂-C₆-alkynyl, C₁-C₆-alkoxy, aryl, heteroaryl, saturated or unsaturated 3-8-membered cycloalkyl, 3-8-membered heterocycloalkyl, C₁-C₆-alkyl aryl, C₁-C₆-alkyl heteroaryl, C₁-C₆-alkenyl aryl, C₁-C₆-alkenyl heteroaryl, C₁-C₆-alkynyl aryl, C₁-C₆-alkynyl heteroaryl, C₁-C₆-alkyl cycloalkyl, C₁-C₆-alkyl heterocycloalkyl, C₁-C₆-alkenyl cycloalkyl, C₁-C₆-alkenyl heterocycloalkyl, C₁-C₆-alkynyl cycloalkyl, or C₁-C₆-alkynyl heterocycloalkyl, or
R³ and R⁴ may form a ring together with the nitrogen to which they are bound; and
R² is selected from the group consisting of H, C₁-C₆-alkyl, C₂-C₆-alkenyl, and C₂-C₆-alkynyl.

4. The benzoxazole acetonitrile according to claim 3, wherein R³ is hydrogen, methyl, ethyl or propyl and R⁴ is a selected from the group consisting of H, (C₁-C₆)-alkyl, C₁-C₆ alkyl-aryl, C₁-C₆-alkyl-heteroaryl, cycloalkyl, heterocycloalkyl, aryl, heteroaryl, 4-8 membered saturated cycloalkyl, and 4-8 membered unsaturated cycloalkyl.

5. The benzoxazole acetonitrile according to claim 3, wherein R³ is H and R⁴ is selected from the group consisting of C₁-C₆ alkyl, 3-8 membered cycloalkyl, 3-8 membered heterocycloalkyl, aryl, heteroaryl, C₁-C₆-alkyl aryl, C₁-C₆ alkyl heteroaryl, C₁-C₆-alkyl cycloalkyl, and C₁-C₆-alkyl heterocycloalkyl.

6. The benzoxazole acetonitrile according to claim 5, wherein R⁴ is selected from the group consisting of C₂-C₄ alkyl substituted with a heteroaryl or heterocycloalkyl group, and C₂-C₄ alkyl substituted with a heteroaryl or heterocycloalkyl-acyl group.

7. The benzoxazole acetonitrile according to claim 6, wherein R⁴ is a propylene-CO-piperazino moiety.

8. The benzoxazole acetonitrile according to claim 1, wherein L is an acylamino moiety of the formula —NR³C(O)R⁴, wherein R³ and R⁴ are each independently from each other H, C₁-C₆-alkyl, C₂-C₆-alkenyl, C₂-C₆-alkynyl, C₁-C₆-alkoxy, aryl, heteroaryl, saturated or unsaturated 3-8-membered cycloalkyl, 3-8-membered heterocycloalkyl, C₁-C₆-alkyl aryl, C₁-C₆-alkyl heteroaryl, C₁-C₆-alkenyl aryl, C₁-C₆-alkenyl heteroaryl, C₁-C₆-alkynyl aryl, C₁-C₆-alkynyl heteroaryl, C₁-C₆-alkyl cycloalkyl, C₁-C₆-alkyl heterocycloalkyl, C₁-C₆-alkenyl cycloalkyl, C₁-C₆-alkenyl heterocycloalkyl, C₁-C₆-alkynyl cycloalkyl, or C₁-C₆-alkynyl heterocycloalkyl.

9. The benzoxazole acetonitrile according to claim 1 selected in the group consisting of:
1,3-benzoxazol-2(3H)-ylidene(2-chloro-6-methylpyrimidin-4-yl)acetonitrile
1,3-benzoxazol-2(3H)-ylidene(2-chloro-6-methylpyrimidin-4-yl)acetonitrile
1,3-benzoxazol-2(3H)-ylidene(6-chloropyrimidin-4-yl)acetonitrile
1,3-benzoxazol-2(3H)-ylidene(2-chloro-5 metylpyrimidin-4-yl)acetonitrile
1,3-benzoxazol-2(3H)-ylidene(2-{[3-(2-oxopyrrolidin-1-yl)propyl]amino)pyrimidin-4-yl)acetonitrile
1,3-benzoxazol-2(3H)-ylidene(2-{[3-(1H-pyrazol-1-yl)propyl]amino}pyrimidin-4-yl)acetonitrile
1,3-benzoxazol-2(3H)-ylidene(2-{[2-(1H-1,2,4-triazol-1-yl)ethyl]amino}pyrimidin-4-yl)acetonitrile
1,3-benzoxazol-2(3H)-ylidene(2-{[2-(1H pyrazol-1-yl)ethyl]amino}pyrimidin-4-yl)acetonitrile
1,3-benzoxazol-2(3H)-ylidene{2-[(2-pyridin-3-ylethyl)amino]pyrimidin-4-yl)acetonitrile
1,3-benzoxazol-2(3H)-ylidene[2-(cyclopropylamino)pyrimidin-4-yl]acetonitrile
1,3-benzoxazol-2(3H)-ylidene(2-{[3-(1H-1,2,4-triazol-1-yl)propyl]amino)pyrimidin-4-yl)acetonitrile
1,3-benzoxazol-2(3H)-ylidene(6-{[3-(3-oxo-4-morpholinyl)propyl]amino}-4-pyrimidinyl)ethanenitrile
1,3-benzoxazol-2(3H)-ylidene(5-methyl-2-{[3-(1H-1,2,4-triazol-1-yl)propyl]amino}-4-pyrimidinyl)ethanenitrile
1,3-benzoxazol-2(3H)-ylidene(5-methyl-2-{[3-(3-oxo-4-morpholinyl)propyl]amino}-4-pyrimidinyl)ethanenitrile
1,3 benzoxazol-2(3H)-ylidene(2-{[3-(3-oxo-4-morpholinyl)propyl]amino}-4-pyrimidinyl)ethanenitrile
1,3-benzoxazol-2(3H)-ylidene(2-{[(2,2-dimethyl-4-oxo-4H-1,3-benzodioxin-6-yl)methyl]amino}-4-pyrimidinyl)ethanenitrile
methyl 5-[({4-[1,3-benzoxazol-2(3H)-ylidene(cyano)methyl]-2-pyrimidinyl}amino)methyl]-2-(2-methoxy-2-oxoethoxy)benzoate
N-[3-({4-[1,3-benzoxazol-2(3H)-ylidene(cyano)methyl]-2-pyrimidinyl}amino)propyl]-2-ethoxy-N-glycoloylacetamide
methyl 4-[2-({4-[1,3-benzoxazol-2(3H)-ylidene(cyano)methyl]-2-pyrimidinyl}amino)ethyl]benzoate
methyl 4-[({4-[1,3-benzoxazol-2(3H)-ylidene(cyano)methyl]-5-methyl-2-pyrimidinyl}amino)methyl]benzoate
methyl{4[({4[1,3 benzoxazol-2(3H)-ylidene(cyano)methyl]-2-pyrimidinyl}amino)methyl]phenoxy}acetate
methyl 5-[({4-[1,3 benzoxazol-2(3H)-ylidene(cyano)methyl]-2-pyrimidinyl}amino)methyl]-2-thiophenecarboxylate
1,3 benzoxazol-2(3H)-ylidene[2-({3-[4-(1-piperidinylsulfonyl)phenyl]propyl}amino)-4 pyrimidinyl]ethanenitrile
ethyl 4-[({4-[1,3-benzoxazol-2(3H)-ylidene(cyano)methyl]-2-pyrimidinyl}amino)methyl]-5-methyl-2-furoate
tert-butyl 4-[({4-[1,3 benzoxazol-2(3H)-ylidene(cyano)methyl]-5-methyl-2-pyrimidinyl}amino)methyl]-1-piperidinecarboxylate
1,3-benzoxazol-2(3H)-ylidene(2-{[3-(1-piperidinylsulfonyl)benzyl]amino}-4-pyrimidinyl)ethanenitrile
methyl 4-[2-({4-[1,3-benzoxazol-2(3H)-ylidene(cyano)methyl]-5-methyl-2-pyrimidinyl}amino)ethyl]benzoate
methyl 4({4-[1,3-benzoxazol-2(3H)-ylidene(cyano)methyl]-2-pyrimidinyl}amino)butanoate
(2-amino-4-pyrimidinyl)(1,3-benzoxazol-2(3H)-ylidene)ethanenitrile
methyl 4[({4-[1,3-benzoxazol-2(3H)-ylidene(cyano)methyl]-2-pyrimidinyl}amino)methyl]benzoate
tert-butyl 4-[({4-[1,3-benzoxazol-2(3H)-ylidene(cyano)methyl]-2-pyrimidinyl}amino)methyl]-1-piperidinecarboxylate
1,3-benzoxazol-2(3H)-ylidene{2-[(2-pyridin-2-ylethyl)amino]pyrimidin-4-yl}acetonitrile
1,3-benzoxazol-2(3H)-ylidene[2-(isopropylamino)pyrimidin-4-yl]acetonitrile
1,3-benzoxazol-2(3H)-ylidene{2-[(2,3-dimethylcyclohexyl)amino]pyrimidin-4-yl)acetonitrile
1,3-benzoxazol-2(3H)-ylidene{2-[(1-methylbutyl)amino]pyrimidin-4-yl)acetonitrile
1,3-benzoxazol-2(3H)-ylidene{2-[(pyridin-2-ylmethyl)amino]pyrimidin-4-yl)acetonitrile
1,3-benzoxazol-2(3H)-ylidene{2-[(3-butoxypropyl)amino]pyrimidin-4-yl}acetonitrile
1,3-benzoxazol-2(3H)-ylidene{2-[(pyridin-3-ylmethyl)amino]pyrimidin-4-yl}acetonitrile
1,3 benzoxazol-2(3H)-ylidene{2-[(3-isopropoxypropyl)amino]pyrimidin-4-yl}acetonitrile
1,3-benzoxazol-2(3H)-ylidene{2-[(1-ethylpropyl)amino]pyrimidin-4-yl}acetonitrile
1,3-benzoxazol-2(3H)-ylidene{2-[ethyl(isopropyl)amino]pyrimidin-4-yl}acetonitrile
1,3-benzoxazol-2(3H)-ylidene[2-(cyclopentylamino)pyrimidin-4-yl]acetonitrile
1,3-benzoxazol-2(3H)-ylidene[2-(cyclohexylamino)pyrimidin-4-yl)acetonitrile
1,3-benzoxazol-2(3H)-ylidene(6-methyl-2-{[3-(1H-1,2,4-triazol-1-yl)propyl]amino}pyrimidin-4-yl)acetonitrile
1,3-benzoxazol-2(3H)-ylidene[2-(cyclopentylamino)-6-methylpyrimidin-4-yl]acetonitrile
1,3-benzoxazol-2(3H)-ylidene[6-(4-ethylpiperazin-1-yl)pyrimidin-4 yl]acetonitrile
1,3-benzoxazol-2(3H)-ylidene[2-(cyclohexylamino)-6-methylpyrimidin-4-yl]acetonitrile
1,3-benzoxazol-2(3H)-ylidene{2-[benzyl(isopropyl)amino]pyrimidin-4-yl}acetonitrile
1,3-benzoxazol-2(3H)-ylidene[6-(cyclopentylamino)pyrimidin-4-yl]acetonitrile
1,3-benzoxazol-2(3H)-ylidene(2-{[4-(4-methyl-1-piperazinyl)-4-oxobutyl]amino}-4-pyrimidinyl)ethanenitrile
1,3-benzoxazol-2(3H)-ylidene(2-{[4-(4-morpholinyl)-4-oxobutyl]amino}-4-pyrimidinyl)ethanenitrile
1,3-benzoxazol-2(3H)-ylidene(2-{[4-oxo-4-(1-piperidinyl)butyl]amino}-4-pyrimidinyl)ethanenitrile
1,3-benzoxazol-2(3H)-ylidene[2-({4-[4-(2-methoxyethyl)-1-piperazinyl]-4-oxobutyl}amino)-4 pyrimidinyl]ethanenitrile
1,3-benzoxazol-2(3H)-ylidene(2-{[4-(1,4-dioxa-8-azaspiro[4.5]dec-8-yl)-4-oxobutyl]amino}-4 pyrimidinyl)ethanenitrile
1,3-benzoxazol-2(3H)-ylidene(2-{[4-oxo-4-(1-piperazinyl)butyl]amino)-4-pyrimidinyl)ethanenitrile
4-[({4-[1,3-benzoxazol-2(3H)-ylidene(cyano)methyl]-5-methyl-2-pyrimidinyl}amino)methyl]benzoic acid
4-[2-({4-[1,3-benzoxazol-2(3H)-ylidene(cyano)methyl]-2-pyrimidinyl}amino)ethyl]benzoic acid
4-[({4-[1,3-benzoxazol-2(3H)-ylidene(cyano)methyl]-2-pyrimidinyl}amino)methyl]benzoic acid
1,3-benzoxazol-2(3H)-ylidene[5-methyl-2-({4-[(4-methyl-1-piperazinyl)carbonyl]benzyl}amino)-4-pyrimidinyl]ethanenitrile
1,3-benzoxazol-2(3H)-ylidene{2-[(2-{4-[(4-methyl-1-piperazinyl)carbonyl]phenyl}ethyl)amino]-4-pyrimidinyl}ethanenitrile
4-[2-({4-[1,3-benzoxazol-2(3H)-ylidene(cyano)methyl]-2 pyrimidinyl}amino)ethyl]-N-[2-(dimethylamino)ethyl]benzamide
1,3-benzoxazol-2(3H)-ylidene[2-({4-[(4-methyl-1-piperazinyl)carbonyl]benzyl}amino)-4-pyrimidinyl]ethanenitrile
1,3-benzoxazol-2(3H)-ylidene{5-methyl-2-[(4-piperidinylmethyl)amino]-4-pyrimidinyl}ethanenitrile
1,3-benzoxazol-2(3H)-ylidene{2-[(4-piperidinylmethyl)amino]-4-pyrimidinyl}ethanenitrile
(2-{[(1-acetyl-4 piperidinyl)methyl]amino}-4-pyrimidinyl)(1,3-benzoxazol-2(3H)-ylidene)ethanenitrile
1,3-benzoxazol-2(3H)-ylidene{2-[({1-[(dimethylamino)acetyl]-4-piperidinyl)methyl)amino]-4 pyrimidinyl}ethanenitrile
(2-{[(1-acetyl-4-piperidinyl)methyl]amino}-5-methyl-4-pyrimidinyl)(1,3-benzoxazol-2(3H)-ylidene)ethanenitrile
N-{4-[1,3-benzoxazol-2(3H)-ylidene(cyano)methyl]-2-pyrimidinyl}-4-(dimethylamino)butanamide
N-{4-[1,3-benzoxazol-2(3H)-ylidene(cyano)methyl]-2-pyrimidinyl}-1-methyl-4-piperidinecarboxamide
1,3-benzoxazol-2(3H)-ylidene{2-[(2-hydroxyethyl)amino]-4-pyrimidinyl}ethanenitrile
methyl 4-({4-[1,3-benzoxazol-2(3H)-ylidene(cyano)methyl]-5-methyl-2-pyrimidinyl}amino)butanoate
1,3-benzoxazol-2(3H)-ylidene(2-{[3-(4-methyl-2-oxo-1-piperazinyl)propyl]amino}-4-pyrimidinyl)ethanenitrile
4-({4-[1,3-benzoxazol-2(3H)-ylidene(cyano)methyl]-2 pyrimidinyl}amino)-N,N-bis(2-methoxyethyl)butanamide
1,3-benzoxazol-2(3H)-ylidene(2-{[4-(4-hydroxy-1-piperidinyl)-4-oxobutyl]amino}-4-pyrimidinyl)ethanenitrile
1,3-benzoxazol-2(3H)-ylidene(2-{[4-(4-isopropyl-1 piperazinyl)-4-oxobutyl]amino)-4-pyrimidinyl)ethanenitrile
1,3-benzoxazol-2(3H)-ylidene(2-{[4-(4-ethyl-1-piperazinyl)-4-oxobutyl]amino}-4-pyrimidinyl)ethanenitrile
1,3-benzoxazol-2(3H)-ylidene(2-{[4-(4-cyclohexyl-1-piperazinyl)-4-oxobutyl]amino}-4-pyrimidinyl)ethanenitrile
1,3-benzoxazol-2(3H)-ylidene(5-methyl-2-{[4-(4-methyl-1-piperazinyl)-4-oxobutyl]amino}-4-pyrimidinyl)ethanenitrile
1,3-benzoxazol-2(3H)-ylidene[2-({4-[4-(2 hydroxyethyl)-1-piperazinyl]-4-oxobutyl}amino)-4 pyrimidinyl]ethanenitrile
1,3-benzoxazol-2(3H)-ylidene(2-{[4-oxo-4-(4 phenyl-1-piperazinyl)butyl]amino}-4-pyrimidinyl)ethanenitrile
4-({4-[1,3-benzoxazol-2(3H)-ylidene(cyano)methyl]-2-pyrimidinyl}amino)-N,N-bis(2-hydroxyethyl)butanamide
1,3-benzoxazol-2(3H)-ylidene[2{{4-oxo-4-[4-(2-pyridinyl)-1-piperazinyl]butyl}amino)-4 pyrimidinyl]ethanenitrile
1,3-benzoxazol-2(3H)-ylidene{2-[(4-oxo-4-{4-[2-oxo-2-(1-pyrrolidinyl)ethyl]-1-piperazinyl}butyl)amino]-4 pyrimidinyl)ethanenitrile
(2-{[4-(4-acetyl-1-piperazinyl)-4-oxobutyl]amino}-4-pyrimidinyl)(1,3-benzoxazol-2(3H)-ylidene)ethanenitrile
ethyl{4-[4-({4-[1,3-benzoxazol-2(3H)-ylidene(cyano)methyl]-2-pyrimidinyl)amino)butanoyl]-1-piperazinyl}acetate
1,3-benzoxazol-2(3H)-ylidene(2-{[4-(4-benzyl-1-piperazinyl)-4-oxobutyl]amino}-4-pyrimidinyl)ethanenitrile
1,3-benzoxazol-2(3H)-ylidene[2-({4-oxo-4-[4-(2-pyrimidinyl)-1-piperazinyl]butyl}amino)-4 pyrimidinyl]ethanenitrile
1,3-benzoxazol-2(3H)-ylidene[2-({4-[4-(2-methoxyethyl)-1-piperazinyl]-4-oxobutyl}amino)-5-methyl-4-pyrimidinyl]ethanenitrile
4-({4-[1,3-benzoxazol-2(3H)-ylidene(cyano)methyl]-2 pyrimidinyl}amino)butanoic acid
1,3-benzoxazol-2(3H)-ylidene(2-{[4-(4-fluoro-1-piperidinyl)-4-oxobutyl]amino}-4-pyrimidinyl)ethanenitrile
2-{4-[({4-[1,3-benzoxazol-2(3H)-ylidene(cyano)methyl]-2-pyrimidinyl}amino)methyl]-1-piperidinyl}-N,N-dimethylacetamide, and
2-{4-[({4-[1,3-benzoxazol-2(3H)-ylidene(cyano)methyl]-5-methyl-2-pyrimidinyl}amino)methyl]-1-piperidinyl}-N,N-dimethylacetamide

10. (canceled)

11. A method of treating at least one disease in a subject in need thereof comprising administering the benzoxazole acetonitrile of claim 1 to the subject in an amount sufficient to treat the at least one disease, wherein the at least one disease is selected from the group consisting of metabolic disorders mediated by insulin resistance, hyperglycemia, diabetes type II, inadequate glucose tolerance, insulin resistance, obesity, polycystic ovary syndrome, and combinations thereof.

12. The method of claim 11, wherein the at least one disease is diabetes type II.

13. A pharmaceutical composition comprising the benzoxazole acetonitrile of claim 1 and a pharmaceutically acceptable carrier, diluent, excipient, or combinations thereof.

14. The composition according to claim 13, further comprising at least one supplementary drug selected from the group consisting of insulin, aldose reductase inhibitors, alpha-glucosidase inhibitors, sulfonyl urea agents, biguanides, thiazolidines, PPARs agonists, GSK-3 inhibitors, and combinations thereof.

15. The composition according to claim 14 wherein the at least one supplementary drug is selected from the group consisting of a rapid acting insulin, an intermediate acting insulin, a long acting insulin, a combination of intermediate and rapid acting insulins, Minalrestat, Tolrestat, Sorbinil, Methosorbinil, Zopolrestat, Epalrestat, Zenarestat, Imirestat, Ponalrestat, ONO-2235, GP-1447, CT-112,1 BAL-ARI 8, AD-5467, ZD5522, M-16209, NZ-314, M-79175, SPR-210, ADN 138, SNK-860, Miglitol, Acarbose, Glipizide, Glyburide, Chlorpropamide, Tolbutamide, Tolazamide, Glimepriride, and combinations thereof.

16. A method of preparing the benzoxazole acetonitrile of formula (I) according to claim 1, comprising reacting a compound of formula (II) with a compound of formula (III) to form the compound of formula (I), wherein A is pyrimidinyl:
L is an amino group, a 3-8 membered heterocycloalkyl comprising at least one heteroatom selected from the group consisting of N, O, and S, or L is an acylamino moiety; and
R¹ is selected from the group consisting of hydrogen, sulfonyl, amino, C₁-C₆-alkyl, C₂-C₆-alkenyl, C₂-C₆-alkynyl, C₁-C₆-alkoxy, aryl, halogen, carboxy, aminocarbonyl, cyano and hydroxy.

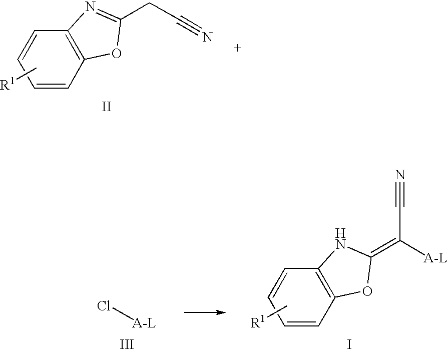

17. A method of forming a compound of formula (Ia), comprising reacting a compound of formula (II) with a compound of formula (III′a) to form the compound of formula (IIa′), and reacting the compound of formula (IIa′) with a compound of formula (IV) to form the compound of formula (Ia), wherein R¹ is selected from the group consisting of hydrogen, sulfonyl, amino, C₁-C₆-alkyl C₂-C₆-alkenyl, C₂-C₆-alkynyl, C₁-C₆-alkoxy, aryl, halogen, carboxy, aminocarbonyl, cyano and hydroxy and
wherein R³ and R⁴ are each independently from each other H, C₁-C₆-alkyl, C₂-C₆-alkenyl, C₂-C₆-alkynyl, C₁-C₆-alkoxy, aryl, heteroaryl, saturated or unsaturated 3-8-membered cycloalkyl, 3-8-membered heterocycloalkyl, C₁-C₆-alkyl aryl, C₁-C₆-alkyl heteroaryl, C₁-C₆-alkenyl aryl, C₁-C₆-alkenyl heteroaryl, C₁-C₆-alkynyl aryl, C₁-C₆-alkynyl heteroaryl, C₁-C₆-alkyl cycloalkyl, C₁-C₆-alkyl heterocycloalkyl, C₁-C₆-alkenyl cycloalkyl, C₁-C₆-alkenyl heterocycloalkyl, C₁-C₆-alkynyl cycloalkyl, or C₁-C₆-alkynyl heterocycloalkyl, or
R³ and R⁴ may form a ring together with the nitrogen to which they are bound; and
R² is selected from the group consisting of H, C₁-C₆-alkyl, C₂-C₆-alkenyl, and C₂-C₆-alkynyl.

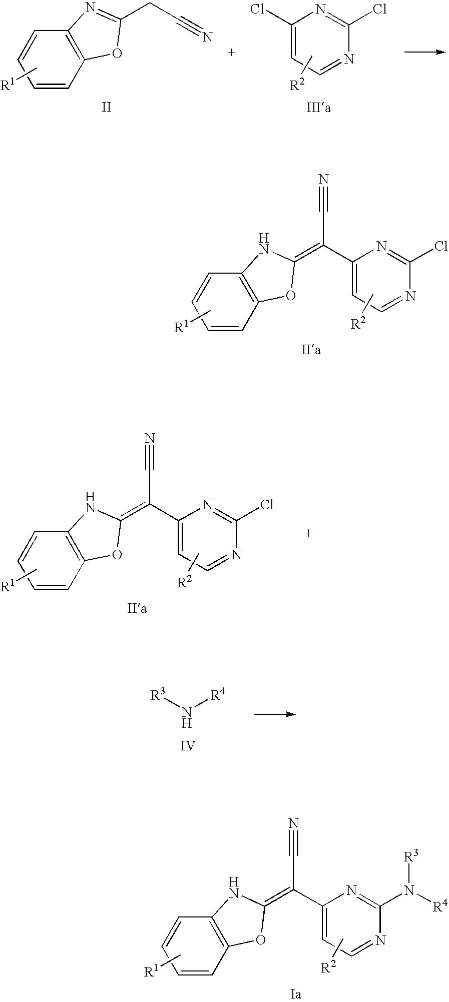

18. A method of preparing a benzoxazole acetonitrile of formula (Ia) of claim 7, comprising reacting a compound of formula (II′a) with a compound of formula (IX) to form a compound of formula (Ic), and reacting the compound of formula (Ic) with a compound of formula (X) to form the benzoxazole acetonitrile of formula (Ia)

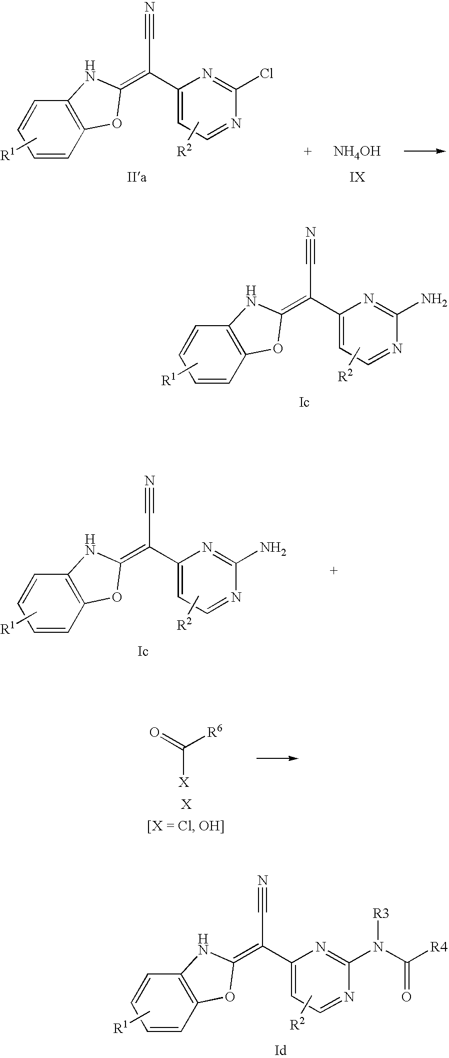

19. An intermediate compound selected from the group consisting of
1,3-benzoxazol-2(3H)-ylidene(2-chloro-6-methylpyrimidin-4-yl)acetonitrile
1,3-benzoxazol-2(3H)-ylidene(2-chloro-6-methylpyrimidin-4-yl)acetonitrile, and
1,3-benzoxazol-2(3H)-ylidene(6-chloropyrimidin-4-yl)acetonitrile.

20. A pharmaceutical composition comprising the benzoxazole acetonitrile of claim 2 and a pharmaceutically acceptable carrier, diluent, excipient, or combinations thereof.

21. A pharmaceutical composition comprising the benzoxazole acetonitrile of claim 3 and a pharmaceutically acceptable carrier, diluent, excipient, or combinations thereof.
